SEQUENCE LISTING (63 sequences)

SEQ ID NO: 1 - DNA, 1482 bp, Unknown
  Novel strain
cttgagagtttgatcctggctcagagcgaacgctggcggcaggcctaacacatgcaagtcgagcgcccagcaatgggagcggcagacgggtgagtaacgcgtggggatgtgcccaatggtacggaataacccagggaaacttggactaataccgtatgagcccttcgggggaaagatttatcgccattggatcaacccgcgtctgattagctagttggtggggtaacggcccaccaaggcgacgatcagtagctggtctgagaggatgatcagccacactgggactgagacacggcccagactcctacgggaggcagcagtggggaatattggacaatgggcgcaagcctgatccagccatgccgcgtgtgtgatgaaggccttagggttgtaaagcactttcgccggtgaagataatgacggtaaccggagaagaagccccggctaacttcgtgccagcagccgcggtaatacgaagggggctagcgttgctcggaatcactgggcgtaaagcgcacgtaggcggatcgttaagtcaggggtgaaatcctggagctcaactccagaactgcccttgatactggcgaccttgagttcgagagaggttggtggaactgcgagtgtagaggtgaaattcgtagatattcgcaagaacaccagtggcgaaggcggccaactggctcgatactgacgctgaggtgcgaaagcgtggggagcaaacaggattagataccctggtagtccacgccgtaaacgatggatgctagccgttgggcagcttgctgttcagtggcgcagctaacgcattaagcatcccgcctggggagtacggtcgcaagattaaaactcaaaggaattgacgggggcccgcacaagcggtggagcatgtggtttaattcgaagcaacgcgcagaaccttaccagcctttgacatggcaggacgatttccagagatggatctcttccagcaatggacctgcacacaggtgctgcatggctgtcgtcagctcgtgtcgtgagatgttgggttaagtcccgcaacgagcgcaaccctcgcctctagttgccagcattcagttgggcactctagagggactgccggtgataagccgagaggaaggtggggatgacgtcaagtcctcatggcccttacgggctgggctacacacgtgctacaatggtggtgacagtgggatgcgaaagggcgacctctagcaaatctccaaaagccatctcagttcggattgtactctgcaactcgagtgcatgaagttggaatcgctagtaatcgtggatcagcatgccacggtgaatacgttcccgggccttgtacacaccgcccgtcacaccatgggagttggctttacccgaaggcgctgcgctaacccgcaagggaggcaggcgaccacggtagggtcagcgactggggtgaagtcgtaacaaggtagccgtaggggaacctgcggctggatcacctccttt

SEQ ID NO: 2 - DNA, 1467 bp, Unknown
  novel strain
atgggtgccgaagcaaccgtcgggcagatcacggacgccaagaagagatacgccgccggcgtgctgaagtacgcccagatgggctactggaacggcgactacgttcccaaggacaccgacctcctggcggtgttccgcatcaccccccaggcgggcgtggacccggtggaagccgccgcggcggtcgccggcgaaagctccaccgctacctggaccgtggtgtggaccgaccggctcaccgccgccgacgtctaccgcgccaaggcctacaaggtggagccggtgccgggccaggaaggccagtatttctgctacatcgcctatgatctcgatttgttcgaggaaggctccatcgccaacctcacggcgtcgatcatcggcaacgtcttctccttcaagccgctgaaggcggcgcggctggaggacatgcggcttcccgtcgcctatgtgaagaccttccgcggcccgcccaccggcatcgtggtcgagcgcgagcgcctggacaagttcggccgcccccttctgggcgccaccaccaagccgaagcttggcctctcgggcaagaattacggccgcgtggtctatgaggccctcaagggcggcctcgacttcgtgaaggacgacgagaacatcaactcgcagcccttcatgcactggcgcgatcgcttcctctattgcatggaggccgtcaacaaggcccaggccgagaccggcgaggtgaaggggcactatctcaacatcaccgccgggaccatggaggagatgtaccgccgcgccgagttcgccaaggaactgggctccgtggtggtgatggtggatctcatcatcggctggaccgccatccagtccatgtccaactggtgccgcgagaacgacatgatcctgcacatgcaccgtgcgggccatggcacctacacgcgccagaagagccacggcgtctccttccgcgtcatcgccaagtggctgcggctcgccggcgtcgaccacctgcacaccggcaccgccgtgggcaagctggaaggcgaccccatgaccgtgcagggcttctacaatgtctgccgcgagacgacgacgcagcaggacctcacccgcggcctgttcttcgagcaggactggggcggcatccgcaaggtgatgccggtggcctccggcggcatccatgcgggccagatgcaccagctcatcgacctgttcggcgaggacgtggtgctccagttcggcggcggcaccatcggccacccggacggcatccaggccggcgccaccgccaaccgcgtggcgctggaaaccatgatcctcgcccgcaacgagggccgcgacatcaggaacgagggcccggaaatcctggtggaagccgccaaatggtgccgtccgctgcgcgcggcgctcgatacctggggcgaggtgaccttcaactacgcctccaccgacacgtccgattacgtgcccaccgcgtccgtcgcctga

SEQ ID NO: 3 - protein, 488 aa, Unknown
  novel strain
MGAEATVGQITDAKKRYAAGVLKYAQMGYWNGDYVPKDTDLLAVFRITPQAGVDPVEAAAAVAGESSTATWTVVWTDRLTAADVYRAKAYKVEPVPGQEGQYFCYIAYDLDLFEEGSIANLTASIIGNVFSFKPLKAARLEDMRLPVAYVKTFRGPPTGIVVERERLDKFGRPLLGATTKPKLGLSGKNYGRVVYEALKGGLDFVKDDENINSQPFMHWRDRFLYCMEAVNKAQAETGEVKGHYLNITAGTMEEMYRRAEFAKELGSVVVMVDLIIGWTAIQSMSNWCRENDMILHMHRAGHGTYTRQKSHGVSFRVIAKWLRLAGVDHLHTGTAVGKLEGDPMTVQGFYNVCRETTTQQDLTRGLFFEQDWGGIRKVMPVASGGIHAGQMHQLIDLFGEDVVLQFGGGTIGHPDGIQAGATANRVALETMILARNEGRDIRNEGPEILVEAAKWCRPLRAALDTWGEVTFNYASTDTSDYVPTASVA

SEQ ID NO: 4 - DNA, 405 bp, Unknown
  novel strain
atgcgcatcacccaaggctccttctccttcctgccggacctcaccgacacgcagatcaaggcccaggtgcaatattgcctggaccagggctgggcggtctcggtggagcacaccgacgatccccacccgcgcaacacctattgggagatgtggggcccgcccatgttcgatctgcgcgacgcggccggcgtcttcggcgagatcgaagcctgccgggccgccaatcccgagcattatgtgcgggtgaacgccttcgattccagccgcggatgggagacgatccgcctgtccttcatcgttcagcggcccaccgtggaagagggcttccgcctcgaccgcaccgaaggcaagggccgcaaccagagctacgccatgcgctaccgggcgcagttcgcgccgcgctga

SEQ ID NO: 5 - protein, 134 aa, Unknown
  novel strain
MRITQGSFSFLPDLTDTQIKAQVQYCLDQGWAVSVEHTDDPHPRNTYWEMWGPPMFDLRDAAGVFGEIEACRAANPEHYVRVNAFDSSRGWETIRLSFIVQRPTVEEGFRLDRTEGKGRNQSYAMRYRAQFAPR

SEQ ID NO: 6 - DNA, 1896 bp, Unknown
  novel strain
atgatgccatctgagccgcacggcgcgggcatgccgcccccacgggaagcggccgcggttcccaccccccaggaggtgagcgcggtggtggccgaggtggtcgcggatgccgtggcatcggtgggcggcgcacgcacccggctcatggacatcgtccagctggcccagcagcgtctcggccatctctccgaagagaccatggcggccattgccgcgcggctcgccattccgccggtggaagtggcggacatggtgtccttctacgccttcctgaaccgcgcgcccaagggccgctaccacatccgcctgtcgcgcagccccatctcgctgatgaagggcgccgaggcggtggctgccgccttctgccagatcctcggcatcgccatgggcgagacctcgcaggatggcgacttcaccctggaatggaccaacgacatcggcatggccgaccaggagccggccgccctcgtcaacggcacggtgatgacgcagctcgcgcccggcgatgcggccatcatcgtcggccggctgcgggcccatcacgcgcccaatgccctgccgctgttccctggagccggcgtggccggctccggcctgccccatgcccggatccgccccagcctggtgatgccgggacagcttctgttccgcgaggaccacacgacgccgggcgccggcatcaaggcggcactcgccctcaccccggacgaagtggtgcagaaggtctccgccgcgcgcctgcgcgggcggggtggcgccggctttcccaccggtctcaaatggaagctctgccgccagtcgcccgccaccacccgccatgtgatctgcaatgcggacgagggcgagcccggcaccttcaaggatcgcgtgctgctcacgcaggcgccgcacctcatgttcgacggcatgaccatcgccggctacgccttgggggcgcgggagggcgtggtctatctgcgcggcgagtacgcctatctgtgggagcctctgcatgcggtcctgcgcgagcgctatgggctcgggctcgccggcgcgaacatcctgggacacgcgggcttcgacttcgacatccgcatccagctgggcgccggcgcctatatctgcggcgaggaatccgcgctggtggaatcgctggaaggcaagcgcggctcgccccgcgaccgcccccccttccccaccgtgcgcggccatctccagcagcccaccgccgtggacaatgtggagaccttcgcctgcgccgcccgcatcctggaggatggcgtggaggcgttcgcgggcatcggcacgcccgaatccgccggcacgaagctcctctcggtgtcgggcgattgcccgcgccccggcgtgtatgaggtgcccttcggcctcacggtgaacgcgctgctcgaccttgtcggcgcgccggacgccgccttcgtgcagatgggtgggccgtccggccaatgcgtggcgccgaaggattacggccgccgcatcgccttcgaggacctgcccaccggcggctcggtgatggtgttcggcccggggcgcgacgtgctcgccatggtgcgcgagttcgcggatttcttcgccggcgaatcctgcggctggtgcacgccctgccgggtgggcaccaccttgctcaaggaagagctggacaagctcctcgccaaccgcgccaccctcgccgacatccgcgcgctggagaccctggccacgaccgtctcccgcaccagccgctgcggcctcggccagacggcgcccaaccccatcctttccaccatgcgcaacctgccggaagcctatgaggcgaggctgaggcccgaagacttcctgccctgggcctcgctcgacgaggcgctgaagcccgccatcgtcatccagggccgcgcgcccgtgccggaggaagaggcatga

SEQ ID NO: 7 - protein, 631 aa, Unknown
  novel strain
MMPSEPHGAGMPPPREAAAVPTPQEVSAVVAEVVADAVASVGGARTRLMDIVQLAQQRLGHLSEETMAAIAARLAIPPVEVADMVSFYAFLNRAPKGRYHIRLSRSPISLMKGAEAVAAAFCQILGIAMGETSQDGDFTLEWTNDIGMADQEPAALVNGTVMTQLAPGDAAIIVGRLRAHHAPNALPLFPGAGVAGSGLPHARIRPSLVMPGQLLFREDHTTPGAGIKAALALTPDEVVQKVSAARLRGRGGAGFPTGLKWKLCRQSPATTRHVICNADEGEPGTFKDRVLLTQAPHLMFDGMTIAGYALGAREGVVYLRGEYAYLWEPLHAVLRERYGLGLAGANILGHAGFDFDIRIQLGAGAYICGEESALVESLEGKRGSPRDRPPFPTVRGHLQQPTAVDNVETFACAARILEDGVEAFAGIGTPESAGTKLLSVSGDCPRPGVYEVPFGLTVNALLDLVGAPDAAFVQMGGPSGQCVAPKDYGRRIAFEDLPTGGSVMVFGPGRDVLAMVREFADFFAGESCGWCTPCRVGTTLLKEELDKLLANRATLADIRALETLATTVSRTSRCGLGQTAPNPILSTMRNLPEAYEARLRPEDFLPWASLDEALKPAIVIQGRAPVPEEEA

SEQ ID NO: 8 - DNA, 1491 bp, Unknown
  novel strain
atgagccggggatcccccgatgccgggaaagaccgcaccatgagcgccaccgacggcaccaccgccccccgcaagatcgtcatcgatccggtgacccgcgtggagggccacggcaaggtcaccatccgcctggatgaagccggcgcggtggaggatgcgcgtttccacatcgtggagttccgcggcttcgagcggttcatccagggccggatgtactgggaagtgccccttatcatccagcggctgtgcggcatctgcccggtgagccaccatctggcggcggcgaaagccatggaccaggtggcgggcgtggaccgcgtaccgcccaccgccgagaaactgcgccggctgatgcattatgggcaggtgctgcaatccaacgctttgcacatcttccacctcgcctcgcccgacctcctgttcggcttcgacgcgccggccgagcagcgcaacatcatcgccgtgctccagcgttatccggagatcggcaaatgggcgatcttcatcaggaagttcggccaggaggtcatcaaggccaccggcgggcgcaagatccatcccaccagcgccattcccggcggggtcaaccagaacctcgccgtggaggaccgcgacgccctgcgcgccaaggtgggcgagatcatcagctggtgcatggcggcgctggaccatcacaaggcctatgtggcggaaaaccgggcgctgcatgacagcttcgccgccttcccctccgccttcatgagcctcgtggggccggatggcggcatggacctttatgacggcaccctgcgggtgatcgatgccgagggcgcccccctcatcgaaggcgcgccgcccgcctcctaccgcgaccacctcatcgaggaggtgcggccctggagctatctgaaattcccccatctgcgcgccttcggccgcgacgatggctggtatcgggtcggccccctcgcccaggtcaattgcgccgcgtccatcgacacgccccgcgccgaggcggcccggcgggacttcatggccgagggcggcggcaagccggtgcatgccaccctcgcttatcactgggcgcggctcatcgtgctggtccattgcgcggagaagatcgaacagctgctgttcgacgacgacctgcaaggctgcgatctgcgtgcggagggcacccggcgcggggaaggcgtcgcctggatcgaggcgccgcgcggcaccctcatccaccattacgaggtggacgagaacgaccaggtgcgccgcgccaacctcatcgtctccaccacccacaataacgaggccatgaaccgcgccgtgcggcaggtggcgaagacggacctttccggtcgcgagatcaccgaagggctgctgaaccatatcgaggtggccatccgcgccttcgacccctgcctgtcctgcgccacccatgcgctgggccagatgccgctgatcgtgacgcttgaagatgcctccggcgcagagatcgcccgcggagtgaaggaatga

SEQ ID NO: 9 - protein, 496 aa, Unknown
  novel strain
MSRGSPDAGKDRTMSATDGTTAPRKIVIDPVTRVEGHGKVTIRLDEAGAVEDARFHIVEFRGFERFIQGRMYWEVPLIIQRLCGICPVSHHLAAAKAMDQVAGVDRVPPTAEKLRRLMHYGQVLQSNALHIFHLASPDLLFGFDAPAEQRNIIAVLQRYPEIGKWAIFIRKFGQEVIKATGGRKIHPTSAIPGGVNQNLAVEDRDALRAKVGEIISWCMAALDHHKAYVAENRALHDSFAAFPSAFMSLVGPDGGMDLYDGTLRVIDAEGAPLIEGAPPASYRDHLIEEVRPWSYLKFPHLRAFGRDDGWYRVGPLAQVNCAASIDTPRAEAARRDFMAEGGGKPVHATLAYHWARLIVLVHCAEKIEQLLFDDDLQGCDLRAEGTRRGEGVAWIEAPRGTLIHHYEVDENDQVRRANLIVSTTHNNEAMNRAVRQVAKTDLSGREITEGLLNHIEVAIRAFDPCLSCATHALGQMPLIVTLEDASGAEIARGVKE

SEQ ID NO: 10 - DNA, 720 bp, Unknown
  novel strain
atgagcgagacccccttcacctttaccgtggacggcatcgcggtcccggccacccccggccagagcgtcatcgaggcgtgcgatgcggcgggcatctatatcccgcgcctgtgccaccacccggacctgccgccggcgggccattgccgggtgtgcacctgcatcatcgacgggcggccggccagcgcctgcaccatgcccgccgccaggggcatggtggtggagaacgagacgcccgctttgctggcggagcggcgcacgctgatcgagatgctgttcgcggaaggcaaccatttctgccagttctgcgaggcgagcggcgattgcgaattgcaggcgctgggctacctgttcggcatggtggccccgcccttcccccatctgtggccgaagcggccggtggatgccagccatccggatatctatatcgaccacaatcgctgcatcctgtgctcgcgctgcgtgcgcgcctcgcgcaccctggacggcaagtccgtgttcggcttcgaggggcgcggcatcgagatgcatctggcggtgaccggcgggcacctggacgacagcgccatcgccgccgccgacagggcggttgagatgtgcccggtgggctgcatcgtcctcaagcgcaccggctaccgcacgccctatggccggcggcgctacgacgccgcgcccatcggctccgacatcaccgcccggcgcggcggcgcgaaggactga

SEQ ID NO: 11 - protein, 239 aa, Unknown
  novel strain
MSETPFTFTVDGIAVPATPGQSVIEACDAAGIYIPRLCHHPDLPPAGHCRVCTCIIDGRPASACTMPAARGMVVENETPALLAERRTLIEMLFAEGNHFCQFCEASGDCELQALGYLFGMVAPPFPHLWPKRPVDASHPDIYIDHNRCILCSRCVRASRTLDGKSVFGFEGRGIEMHLAVTGGHLDDSAIAAADRAVEMCPVGCIVLKRTGYRTPYGRRRYDAAPIGSDITARRGGAKD

SEQ ID NO: 12 - DNA, 543 bp, Unknown
  novel strain
atggccaagcccaaactcgccacctgcgcgctggccggctgcttcggctgccacatgtccttcctggacatggacgagcgcatcgtcgagctcatcgacctggtggacctcgacgtctcgcccctcgacgacaagaaaaacttcaccggcatggtggaaatcggcctggtggaaggcggctgcgccgacgagcgccatgtgaaggtgctgcgcgagttccgcgagaaatcccgcatcctggtggcggtgggcgcctgcgccatcaccggcggcatcccggcattgcgcaacctcgccggcctcgacgaatgcctgagggaagcctacctcaccggccccacggtggaaggcggcgggctcattcccaacgacccggagctgccgctgctgctggacaaggtctatccggtgcaggacttcgtgaagatcgaccatttcctgcccggctgcccgccctcggccgacgccatctgggcggctctgaaggcgctgctgaccggcaccgagccgcatctgccctacccgcttttcaagtacgaatga

SEQ ID NO: 13 - protein, 180 aa, Unknown
  novel strain
MAKPKLATCALAGCFGCHMSFLDMDERIVELIDLVDLDVSPLDDKKNFTGMVEIGLVEGGCADERHVKVLREFREKSRILVAVGACAITGGIPALRNLAGLDECLREAYLTGPTVEGGGLIPNDPELPLLLDKVYPVQDFVKIDHFLPGCPPSADAIWAALKALLTGTEPHLPYPLFKYE

SEQ ID NO: 14 - DNA, 1013 bp, Unknown
  novel strain
tccagacccgggcaacattgctccatgtgctgggcaccctggccggccgctggccccataccctcgcgctccagcccggcggggtgacccgaagcgccgaccagcacgaccgcatgcgcctgctcgcgacgctgaaggcggtgcgggcggcgctggaagagaccttgttcggcgcgcctttggaagaggtggcggccctggacggcgccgccgccgtggaggcctggcgcgccaacggcccggaaggggatttccgcctgttcctggagatcgccgccgacctggagctggaccggctcggccgcgcgcacgaccgctttctctccttcggcgcctacgcccaggacgaggggcgcctttatggcgccggcaccttcgaggccgggacggcgggagggctcgatcccaacgccatcaccgaggaccacgccttcgcccgcatggaggaccgcgcggcgccccatgcgccctttgacggctccaccttccccgatgccgacgacaccgagggctacacctggtgcaaggcgccgcgccttgccggcctgcccttcgagaccggcgccttcgcccggcaggtggtggcgggccatccgctcgcccgggacctcgtgacgcgggaaggcggcactgtgcgcagccgcgtggtcggccggctgctggaaaccgcgcgcaccctgatcgccatggagggctgggtgaaggaactgcggcccgaagggccctggtgcgcccagggccacctgccccaggaaggccgcgccttcggcctcaccgaggcggcgcgcggggcgctcggccactggatggtggtggagaagggccgcattgcccgctaccagatcatcgcccccaccacctggaacttctccccccgcgacggcgcgggcctgcccggcccgctggagacggccctggtgggcgcgcccgtgcggcagggagagacgacgcccgtgagcgtgcagcacatcgtgcgctccttcgacccgtgcatggtctgcactgtgcattga

SEQ ID NO: 15 - protein, 485 aa, Unknown
  novel strain
MSAETRRLVVGPFNRVEGDLEVRLDVQDGRVQQAFVSSPLFRGFERILEGRDPRDALVIAPRICGICSVSQSHAAALALAGLQGIAPTHDGRIATNLIVAAENVADHLTHFHVFFMPDFARAVYEDRPWFAQAARRFKANQGVSVRRALQTRATLLHVLGTLAGRWPHTLALQPGGVTRSADQHDRMRLLATLKAVRAALEETLFGAPLEEVAALDGAAAVEAWRANGPEGDFRLFLEIAADLELDRLGRAHDRFLSFGAYAQDEGRLYGAGTFEAGTAGGLDPNAITEDHAFARMEDRAAPHAPFDGSTFPDADDTEGYTWCKAPRLAGLPFETGAFARQVVAGHPLARDLVTREGGTVRSRVVGRLLETARTLIAMEGWVKELRPEGPWCAQGHLPQEGRAFGLTEAARGALGHWMVVEKGRIARYQIIAPTTWNFSPRDGAGLPGPLETALVGAPVRQGETTPVSVQHIVRSFDPCMVCTVH

SEQ ID NO: 16 - DNA, 858 bp, Unknown
  novel strain
acgggggaggaagcccgcgccatcttcgacgccatccttgccggcgttatcgtcctcgacgccctgtgcgtggaaggcgcgctgctgcgcgggccgaacggcaccgggcgcttccatgtgctggcgggcacggacacccccaccatcgactgggcgcggcagctcgccggcatggcgcgccacgtggtggcggtgggcacctgcgccgcctatgggggcgtgacggcggcgggcatcaaccccaccgatgcctgcggcctccagttcgacggacgccggaagggtggggcgctgggggcggacttccgctcccgctcggggcttccggtcatcaatgtggccggctgccccacccatcccaactgggtgacggaaaccctgatgctgctcgcctgcggcctgctgggcgaggccgacctcgacgtctatggccgcccgcgcttctatgcggacctgctggtgcatcacggctgcccgcgcaacgaatactatgaatacaaggcgagcgccgagaagatgagcgacctcggctgcatgatggagcatctgggctgcctcggcacccaggcccacgccgactgcaacacgcgcctttggaatggcgagggctcgtgcacccgcggcggctatgcctgcatcaactgcacggcgccggaattcgaggagccgggccacgccttcctggagacgcccaagatcggcggcatccccatcggcctgcccaccgacatgcccaaggcctggttcatcgccttgtcctccctcgccaaggcggcgacgccggagcggctgcgcaagaacgcggtgtccgaccatgtggtcacgccgcccgccgtcaaggacatcaagcggcgatga

SEQ ID NO: 17 - protein, 335 aa, Unknown
  novel strain
MSTPFSVLWLQSGGCGGCTMSLLCAEAPDLATTLDAAGIGFLWHPALSEETGEEARAIFDAILAGVIVLDALCVEGALLRGPNGTGRFHVLAGTDTPTIDWARQLAGMARHVVAVGTCAAYGGVTAAGINPTDACGLQFDGRRKGGALGADFRSRSGLPVINVAGCPTHPNWVTETLMLLACGLLGEADLDVYGRPRFYADLLVHHGCPRNEYYEYKASAEKMSDLGCMMEHLGCLGTQAHADCNTRLWNGEGSCTRGGYACINCTAPEFEEPGHAFLETPKIGGIPIGLPTDMPKAWFIALSSLAKAATPERLRKNAVSDHVVTPPAVKDIKRR

SEQ ID NO: 18 - DNA, 870 bp, Unknown
  novel strain
gtgaccgagcgcctgtccgacgtcaacgcccgcatcgcctcggtgcggcagctctcatcggtcatcacggccatgcggggcattgcggcggcgcgggcgcgggaggcgcggggtcggctcgacggcatccgcgcctatgcgcagaccatcgccgaggccatcggccatgtgctcgccgtgctgcccgaggaggcccgcgcccggtcctccgggcaccggcatcggggccatgcggtcatcgccctgtgcgcggagcagggctttgccggcgtcttcaacgagcgggtgctggacgaggccgcccggctgctgaccggcggggcggggccggccgagctgctgctggtgggcgaccggggcctgatggtggcccgcgagcgggggctcgatgtctcctggtcggtgcccatggtggcccatgcgggccaggcctcggcgctggcggaccgcatcagcgaggagctctaccggcggatcgatgcgggacgggtgacgcgggtgtcggtggtgcacgccgagcccgccgcgtccgccgccatcgagacggtggtgaaagtgctggtgccgttcgacttcgcccgcttccccctggcgcgggtggcatccgccccgctcatgaccatgccgccgccgcggctgctggcccagctgtcggaggaatatgtgttcgccgagctgtgcgaggcgctcaccttgtccttcgcggcggagaacgaggcccgcatgcgggccatgatcgccgcccgcgccaatgtggccgataccctggagggcctcgtcggccgcgcccggcagatgcgccaggaggagatcaccaacgagatcatcgagctggaaggcggcgccggcagcgcccggcatgcggattga

SEQ ID NO: 19 - protein, 289 aa, Unknown
  novel strain
MTERLSDVNARIASVRQLSSVITAMRGIAAARAREARGRLDGIRAYAQTIAEAIGHVLAVLPEEARARSSGHRHRGHAVIALCAEQGFAGVFNERVLDEAARLLTGGAGPAELLLVGDRGLMVARERGLDVSWSVPMVAHAGQASALADRISEELYRRIDAGRVTRVSVVHAEPAASAAIETVVKVLVPFDFARFPLARVASAPLMTMPPPRLLAQLSEEYVFAELCEALTLSFAAENEARMRAMIAARANVADTLEGLVGRARQMRQEEITNEIIELEGGAGSARHAD

SEQ ID NO: 20 - DNA, 1536 bp, Unknown
  novel strain
atgagcacgggcgcgcaagcgagcgaggattggctcacccggagccgggcggccctggccgggacgcgcctttcccagcaatcccaatcggtgggccgggtggaggagatggccgacggcatcgcccgcgtctccggcctgccggatgtgcggctcgacgagcttctcaccttcgagggcggccagaccggctatgccctcaccctcgatcgcaccgagatcgccgtggtgctgctggatgacgcctccggcgtggaggcgggcgcccgggtgttcggcaccggcgaggtggtgaaggtgccggtggggccggggctgctgggccgcatcgtcgaccccctcggccggcccatggaccgctccgagccggtggtggcgcaggcgcaccatcccatcgagcggccggcgccggccatcatcgcccgcgacctggtctcgcagccggttcagaccggcacgctggtggtggatgcgctgttctccctcggccggggccagcgcgagctcatcatcggcgaccgggctaccggcaagaccgccatcgcggtggacaccatcatcagccagaagcattcggacatcgtgtgcatctacgtggcggtgggccagcgcgccgccgccgtggagcgggtggtggaggcggtgcgcgcccacggggcgatcgagcgctgcatcttcgtggtcgcctcggccgccgcctcgccagggctgcaatggatcgcgccgttcgccggcatgaccatggcggaatatttccgcgacaacggccagcatgcgctcatcatcatcgatgatctcaccaagcatgcggccacccatcgcgagctggcgctgctcacccacgagccgccgggccgcgaggcctatcccggcgacatcttctatgtgcacgcccgccttctggagcgggccgccaagctctccgccgagctgggcggtggctcgctcacggccctgcccatcgcggagacggacgcgggaaacctctccgcctatatccccaccaacctcatctccatcaccgatgggcagatcgtgctggattcgcggctgttcgcggccaaccagcgcccggcggtggatgtgggcctctccgtgagccgggtgggcggcaaggcgcagcatcccgcgcttcgggccgtgtccgggcgcatccggctcgattattcccagttcctggagctggaaatgttcacccgcttcggcggcatcaccgatacccgcgtgaaggcgcagatcacccggggcgagcgcatccgcgcgctgctcacccagccgcgcttttccaccctgcgccttcaggacgaggtggcgctgctggccgcgctggcggagggggtgttcgacactttggccccggggctgatgggcgccgtgcgtgcccgcattccggcccagctggatgcgcaggtgaaggacgtggcctcggccctcgccgagggcaaggtgctggaggagggcttgcacgcccgtctcgtggcggccgtgcgggccgtcgcggcggacgtggccgcgaccgcgaaggccgggccgtga

SEQ ID NO: 21 - protein, 511 aa, Unknown
  novel strain
MSTGAQASEDWLTRSRAALAGTRLSQQSQSVGRVEEMADGIARVSGLPDVRLDELLTFEGGQTGYALTLDRTEIAVVLLDDASGVEAGARVFGTGEVVKVPVGPGLLGRIVDPLGRPMDRSEPVVAQAHHPIERPAPAIIARDLVSQPVQTGTLVVDALFSLGRGQRELIIGDRATGKTAIAVDTIISQKHSDIVCIYVAVGQRAAAVERVVEAVRAHGAIERCIFVVASAAASPGLQWIAPFAGMTMAEYFRDNGQHALIIIDDLTKHAATHRELALLTHEPPGREAYPGDIFYVHARLLERAAKLSAELGGGSLTALPIAETDAGNLSAYIPTNLISITDGQIVLDSRLFAANQRPAVDVGLSVSRVGGKAQHPALRAVSGRIRLDYSQFLELEMFTRFGGITDTRVKAQITRGERIRALLTQPRFSTLRLQDEVALLAALAEGVFDTLAPGLMGAVRARIPAQLDAQVKDVASALAEGKVLEEGLHARLVAAVRAVAADVAATAKAGP

SEQ ID NO: 22 - DNA, 741 bp, Unknown
  novel strain
atgcagatcgactggtggacgctgggcctgcagacggtcaacgtcctcgttctcatctggctcctgagccgcttcctgttcaagccggtggcgcaggtcatcgcgcagcgccgtgccgagatcgagaagctggtggaggatgcgcgcgccgccaaggccgccgccgaggccgagcgggacacggcgaaggcggaggaggcgcgccttgccgccgagcgcggcgcccgcatggcggcggtcgccaaggaggcggaggcgcagaaggcggcattgctggccgccgccaagaccgaggccgaggccctgcacgcggccgcggaagcggccatcgtccgggcgcgggcgagcgaggaggaagccgccgccgaccgcgccagccgccttgccgtggacatcgccgccaagctgctggaccggctgcccgacgacgcccgggtcgcgggcttcatcgatggcctcgccgaggggcttgaagccctgcccgaggcgagccgggcggtgatcggcgtcgacggcgcgccagtgcgcgtgacggccgcgcgcgcccttatgccggcggaggaggaggcctgccgcacgcggctctcccaggcgctgggccgtccggtgacgctggccgtgaccatcgaccccgccctcatcgccggcctggagatggagacgccccacgcggtggtgcgcaattccttcaaggccgatctcgaccgcgtcaccgcggcgctcacccatcatgggacctga

SEQ ID NO: 23 - protein, 246 aa, Unknown
  novel strain
MQIDWWTLGLQTVNVLVLIWLLSRFLFKPVAQVIAQRRAEIEKLVEDARAAKAAAEAERDTAKAEEARLAAERGARMAAVAKEAEAQKAALLAAAKTEAEALHAAAEAAIVRARASEEEAAADRASRLAVDIAAKLLDRLPDDARVAGFIDGLAEGLEALPEASRAVIGVDGAPVRVTAARALMPAEEEACRTRLSQALGRPVTLAVTIDPALIAGLEMETPHAVVRNSFKADLDRVTAALTHHGT

SEQ ID NO: 24 - DNA, 243 bp, Unknown
  novel strain
atgactgtcgagatggtcagcatcttcgcggcggcgctcgccgtctccttcggcgccatcgggccggccctgggcgagggccgggcggtggccgcggccatggacgccatcgcccgccagccggaggcggccggaaccttgtcgcgcacgctcttcgtcggcctcgccatgatcgagaccatggcgatctactgcctggtgatcgcgctcctggtgctcttcgccaatccgttcgtgaagtga

SEQ ID NO: 25 - protein, 80 aa, Unknown
  novel strain
MTVEMVSIFAAALAVSFGAIGPALGEGRAVAAAMDAIARQPEAAGTLSRTLFVGLAMIETMAIYCLVIALLVLFANPFVK

SEQ ID NO: 26 - DNA, 699 bp, Unknown
  novel strain
atgggctcgccgctgatcctcgaacccctgttccatatcgggcccgtgcccatcaccgcgccggtggtggtcacctggctcatcatggccgccttcattgggctggcgcggctcatcacccggaagctttccaccgatcccacccggacccaggcggcggtggaaacggtgctgaccgccatcgattcccagatcgccgacaccatgcaggccgatcccgcgccttatcgcgcgctcatcggcaccatcttcctttatgtgctggtggccaactggtcctcgctcatcccgggcatcgagccgcccacggcgcatatcgagaccgatgcggcgctcgctttcatcgtgttcgccgccaccatcgggttcgggttgaagacaaggggtgtgaagggctatctcgccaccttcgccgaaccctcctgggtgatgatcccgctcaatgtggtggagcagatcacccggaccttctcgctcatcgtgcgcctgttcggcaacatcatgagcggggtgttcgtggtcggcatcatcctgtccctcgccgggctgctggtgcccatccccctcatggcgctcgatctcctgaccggcgccgtgcaggcctacatcttcgcggtgctggcctgcgtgttcatcggcgcggccattggcgaggcgccggcaaagccccaatcgaaggagccagggaaaacatcatga

SEQ ID NO: 27 - protein, 232 aa, Unknown
  novel strain
MGSPLILEPLFHIGPVPITAPVVVTWLIMAAFIGLARLITRKLSTDPTRTQAAVETVLTAIDSQIADTMQADPAPYRALIGTIFLYVLVANWSSLIPGIEPPTAHIETDAALAFIVFAATIGFGLKTRGVKGYLATFAEPSWVMIPLNVVEQITRTFSLIVRLFGNIMSGVFVVGIILSLAGLLVPIPLMALDLLTGAVQAYIFAVLACVFIGAAIGEAPAKPQSKEPGKTS

SEQ ID NO: 28 - DNA, 456 bp, Unknown
  novel strain
gtgagcgcgccgctgcacctcaccatcaccacgccggccgccgttctggtggaccgtgccgacatcgtggccctgcgtgccgaggacgagagcggcagcttcggcatcctgcccggccatgcggatttcctgaccgttctggaggcctgcgtggtgcgcttcaaggatggggccgacggcgtgcattattgtgctctcagtggtggcgtgctgtcggtcgaggagggccggcgcatcgccatcgcctgccgtcagggcacggtgagcgacgacctggtcgccctggaaggggcggtggacgccatgcgttcggcggagagcgatgccgacaagcgggcccgggtggagcagatgcgccttcatgcccacgccgtgcgccagctcctgcactatctgcggcccggccgggccggcggcgtggcgccggccgccgcgccggaggaggggccgtcatga

SEQ ID NO: 29 - protein, 151 aa, Unknown
  novel strain
MSAPLHLTITTPAAVLVDRADIVALRAEDESGSFGILPGHADFLTVLEACVVRFKDGADGVHYCALSGGVLSVEEGRRIAIACRQGTVSDDLVALEGAVDAMRSAESDADKRARVEQMRLHAHAVRQLLHYLRPGRAGGVAPAAAPEEGPS

SEQ ID NO: 30 - DNA, 1494 bp, Unknown
  novel strain
atggcagcggcagatgaggaggcgcaatcggccgccggccccgcctcgggccgggtggtggccgtgcgcggcgcggtgatcgacatcgcctttgcccagcctccgctgccgccgctggacgacgcccttctcatcaccgacggccggggcggcacggtgctggtggaggtgcagagccatatggatcggcacacggtgcgcgccatcgcccttcaggccaccaccggcctcagccgggggctggaggcggcgcgggtgggcgggccggtgaaggtgccggtgggagaccatgtgctcggccgcctcctggatgtcaccggcgccatcggcgacaagggcgggccgctgccggccgacgtgcccacgcggccgatccaccacgcgccgccatccttcgccgcgcagggcggcacgtccgatctgtttcgcaccggcatcaaggtcatcgacctcctggcgcccctcgcccagggcggcaaggcggccatgttcggcggggccggcgtgggcaagaccgtgctggtgatggagctgatccacgccatggtggcgagctacaagggcatctcggtgtttgccggcgtgggggagcgctcccgcgagggccacgagatgctgctggacatgaccgattccggcgtgctcgaccgcaccgttctggtctatggccagatgaacgagccccccggggcccgctggcgggtgcccatgacggcgctgaccatcgccgaatatttccgcgacgagaagcaccagaacgtcctgctgctgatggacaacatcttccgcttcgtccaggcgggggcggaggtctccggccttttgggccgtccgccctcccgggtgggataccagccgacgctggcgagcgaggtggcggcgctccaggaacgcatcacctccgtgggcgaggcctcggtgaccgccatcgaggcggtctacgtgccggcggatgacttcaccgatcccgccgtgaccaccatcgccgcccacgtggattccatggtggtgctctcccgcgccatggcggcggagggcatgtatccggcggtggaccccatctcctcctcgtcggtgctgctcgacccgctcatcgtgggggacgagcatgcgcgcgtcgccaacgaggtgcgccggaccatcgagcattatcgcgagcttcaggatgtgatctcgctgctgggcatggaggaattgggcaccgaggatcgccgcatcgtggagcgggcgcgccggctccagcgcttcctcacccagcccttcacggtcaccgaggccttcaccggcgtgcccggccgctcggtggccatcgccgacaccatcgccggctgcaggatgatcctgtccggcgcctgcgacgactggcaggaaagcgccctctacatggtgggcaccatcgacgaggcccgccagaaggaggaggccgctcgcgccaaggcggggcagggcgccccggccgggacggcagccgagacggcggaggccgccccgtga

SEQ ID NO: 31 - protein, 497 aa, Unknown
  novel strain
MAAADEEAQSAAGPASGRVVAVRGAVIDIAFAQPPLPPLDDALLITDGRGGTVLVEVQSHMDRHTVRAIALQATTGLSRGLEAARVGGPVKVPVGDHVLGRLLDVTGAIGDKGGPLPADVPTRPIHHAPPSFAAQGGTSDLFRTGIKVIDLLAPLAQGGKAAMFGGAGVGKTVLVMELIHAMVASYKGISVFAGVGERSREGHEMLLDMTDSGVLDRTVLVYGQMNEPPGARWRVPMTALTIAEYFRDEKHQNVLLLMDNIFRFVQAGAEVSGLLGRPPSRVGYQPTLASEVAALQERITSVGEASVTAIEAVYVPADDFTDPAVTTIAAHVDSMVVLSRAMAAEGMYPAVDPISSSSVLLDPLIVGDEHARVANEVRRTIEHYRELQDVISLLGMEELGTEDRRIVERARRLQRFLTQPFTVTEAFTGVPGRSVAIADTIAGCRMILSGACDDWQESALYMVGTIDEARQKEEAARAKAGQGAPAGTAAETAEAAP

SEQ ID NO: 32 - DNA, 1437 bp, Unknown
  novel strain
atggcgaacaaggtcggacgcatcacccagatcatcggcgccgtcgtcgacgtgcagttcgacgggcatctgccggcgattctcaacgcgatcgagaccaccaaccagggcaaccggctggtgctcgaagtggctcagcatctcggcgagaacaccgtgcgctgcatcgccatggatgccactgaaggcctggtgcgtggccaggaggtggccgacaccgatgcgcccatccaggtgcccgtgggcgccgccaccctcggccgcatcatgaacgtgatcggcgagccggtggacgagctgggccccatcgagggcgaagcgctgcgcggcatccatcagccggccccctcctatgcggagcaggccacggaagctgagatcctcgtcaccggcatcaaggtggtggatctgctggcgccctattccaagggcggcaaggtgggcctgttcggcggcgccggcgtgggcaagaccgtgctcatcatggagctgatcaacaacgtggccaaggcgcacggcggctattccgtgttcgccggcgtgggtgagcgcacccgcgagggcaacgacctctaccacgagatgatcgagtccaacgtgaacaaggacccgcacgagaacaatggctcggcggccggttccaagtgcgccctggtctatggccagatgaacgagccgcccggcgcccgcgcccgcgtggccctcaccggcctcaccgtcgccgagcatttccgcgaccagggccaggacgtgctgttcttcgtggacaacatcttccgcttcacccaggcgggctccgaggtgtcggcgcttctcggccgcatcccctcggcggtgggctaccagccgacgctggccaccgacatgggccagctgcaggagcgcatcaccaccaccaccaagggctccatcacctcggtgcaggccatctacgtgccggcggacgatctgaccgatccggcgccggccgcctccttcgcccatctggacgccaccacggtgctgtcgcgctccatcgcggagaagggcatctacccggcggtggatccgctggactccacctcgcgcatgctgtctcccgccatcctcggcgacgagcactacaacaccgcgcgccaggtgcagcagaccctgcagcgctacaaggcgctccaggacatcatcgccatcctgggcatggacgaactctccgaagaggacaagctcaccgtggcccgcgcccgcaagatcgagcgcttcctctcccagcccttccacgtggccgaggtgttcaccggttcgcccggcaagctggtcgacctcgccgacaccatcaagggcttcaagggcctggtggacggcaagtacgactacctgcccgagcaggccttctacatggtgggcaccatcgaagaagccatcgagaagggcaagaagctggcggccgaggcggcctga

SEQ ID NO: 33 - protein, 478 aa, Unknown
  novel strain
MANKVGRITQIIGAVVDVQFDGHLPAILNAIETTNQGNRLVLEVAQHLGENTVRCIAMDATEGLVRGQEVADTDAPIQVPVGAATLGRIMNVIGEPVDELGPIEGEALRGIHQPAPSYAEQATEAEILVTGIKVVDLLAPYSKGGKVGLFGGAGVGKTVLIMELINNVAKAHGGYSVFAGVGERTREGNDLYHEMIESNVNKDPHENNGSAAGSKCALVYGQMNEPPGARARVALTGLTVAEHFRDQGQDVLFFVDNIFRFTQAGSEVSALLGRIPSAVGYQPTLATDMGQLQERITTTTKGSITSVQAIYVPADDLTDPAPAASFAHLDATTVLSRSIAEKGIYPAVDPLDSTSRMLSPAILGDEHYNTARQVQQTLQRYKALQDIIAILGMDELSEEDKLTVARARKIERFLSQPFHVAEVFTGSPGKLVDLADTIKGFKGLVDGKYDYLPEQAFYMVGTIEEAIEKGKKLAAEAA

SEQ ID NO: 34 - DNA, 876 bp, Unknown
  novel strain
atggcgagtctgaaggacctgagaaaccgcattgcctcggtgaaggcgacgcagaagatcaccaaggcgatgcagatggtcgccgcggcgaagctgcgtcgcgcccaggcggcggctgaagcggcccgtccctatgcggaacgcatggagacggtgctcggaaatcttgcctccggcatggtggtgggcgcgcaggcgcctgttctcatgaccgggacgggcaagagcgacacccacctgctgctggtgtgcaccggcgagcgcggcctgtgcggcgccttcaactcgtccatcgtgcgcttcgcccgcgagcgggcgcagctgctgctggccgagggcaagaaggtgaaaatcctgtgcgtgggccgcaagggccacgagcagctgcgccgcatctacccggacaacatcatcgacgtggtggacctgcgcgcggtgcgcaacatcggcttcaaggaggccgacgccatcgcccgcaaggtgctggccctgctcgatgaaggcgcattcgacgtctgcacgctcttctactcccacttcaggagcgtgatcgcccaggtgccgacggcccagcagctcattccggccaccttcgacgagcggccggccgtcgccgatgcgccggtctatgaatatgagccggaggaggaggagatcctcgccgagctgctgccgcgcaacgtggcggtgcagatcttcaaggccctcctcgagaaccaggcttctttctatggctcccagatgagcgccatggacaacgccacgcgcaatgcgggcgagatgatcaagaagcagacgctcacctacaaccgtacccgccaggccatgatcacgaaggaactcatcgagatcatctccggcgccgaggccgtctga

SEQ ID NO: 35 - protein, 291 aa, Unknown
  novel strain
MASLKDLRNRIASVKATQKITKAMQMVAAAKLRRAQAAAEAARPYAERMETVLGNLASGMVVGAQAPVLMTGTGKSDTHLLLVCTGERGLCGAFNSSIVRFARERAQLLLAEGKKVKILCVGRKGHEQLRRIYPDNIIDVVDLRAVRNIGFKEADAIARKVLALLDEGAFDVCTLFYSHFRSVIAQVPTAQQLIPATFDERPAVADAPVYEYEPEEEEILAELLPRNVAVQIFKALLENQASFYGSQMSAMDNATRNAGEMIKKQTLTYNRTRQAMITKELIEIISGAEAV

SEQ ID NO: 36 - DNA, 1530 bp, Unknown
  novel strain
atggacattcgagccgctgaaatctctgccatcctgaaagagcagatccagaatttcggccaggaggcggaagtctccgaggtgggtcaggttctgtccgtgggtgacggcatcgcgcgcgtctacggcctcgacaacgtccaggcgggcgagatggtcgagttcgagaacggcacgcgcggcatggcgctgaacctcgagctcgacaatgtcggcatcgtgatcttcggttccgaccgcgagatcaaggaaggccagaccgtcaagcggaccggcgccatcgtggacgcccccgtcggcaagggcctgctcggccgcgtcgtggacgctctcggcaacccgatcgacggcaagggcccgatcatgttcaccgagcgtcgccgggtcgacgtgaaggcgccgggcatcatcccgcgcaagtcggtgcacgagcccatgcagaccggcctgaaggccatcgatgcgctcatccccatcggccgcggccagcgcgagctcatcatcggcgaccgccagaccggcaagaccgccgtggcgctcgactcgatcctgaaccagaagcccatcaaccagggcgacgacgagaaggccaagctctactgcgtctatgtcgcggtgggccagaagcgttccactgtcgcgcagttcgtgaaggtgctcgaggagcacggcgcgctggaatattccatcgtcgtcgccgccaccgcctcggacgcggcccccatgcagttcctggcgccgttcaccggcaccgccatgggcgagtatttccgcgacaacggcatgcacgccctcatcatccatgatgacctgtccaagcaggccgtggcctaccgccagatgtcgctgctgctgcgccgcccgccgggccgcgaggcctatcccggcgatgtgttctacctgcactcccgcctcttggagcgcgccgccaagctcaatgacgagcacggcgccggctcgctgaccgccctgccggtgatcgagacccaggccaacgacgtgtcggcctacatcccgaccaacgtgatctccatcaccgacggtcagatcttccttgaatccgatctgttctaccagggcatccgcccggcggtgaacgtgggcctgtcggtgtcgcgcgtgggctcttcggcccagatcaaggcgatgaagcaggtggccggcaagatcaagggcgagctcgcccagtatcgcgagctggcggccttcgcccagttcggttcggacctggacgcggccacccagaagctgctgaaccgcggcgcccgcctcaccgagctgctgaagcagagccagttctcgcccctcaaggtggaggagcaggtggcggtgatctatgccggcaccaatggctatctcgatccgctgccggtctccaaggtgcgcgagttcgagcagggtctgctcctgtcgctgcgctcgcagcatccggagatcctggacgccatccgcacgtccaaggagctttccaaggacaccgccgagaagctgacgaaggccatcgacgccttcgccaagagcttctcctga

SEQ ID NO: 37 - protein, 509 aa, Unknown
  novel strain
MDIRAAEISAILKEQIQNFGQEAEVSEVGQVLSVGDGIARVYGLDNVQAGEMVEFENGTRGMALNLELDNVGIVIFGSDREIKEGQTVKRTGAIVDAPVGKGLLGRVVDALGNPIDGKGPIMFTERRRVDVKAPGIIPRKSVHEPMQTGLKAIDALIPIGRGQRELIIGDRQTGKTAVALDSILNQKPINQGDDEKAKLYCVYVAVGQKRSTVAQFVKVLEEHGALEYSIVVAATASDAAPMQFLAPFTGTAMGEYFRDNGMHALIIHDDLSKQAVAYRQMSLLLRRPPGREAYPGDVFYLHSRLLERAAKLNDEHGAGSLTALPVIETQANDVSAYIPTNVISITDGQIFLESDLFYQGIRPAVNVGLSVSRVGSSAQIKAMKQVAGKIKGELAQYRELAAFAQFGSDLDAATQKLLNRGARLTELLKQSQFSPLKVEEQVAVIYAGTNGYLDPLPVSKVREFEQGLLLSLRSQHPEILDAIRTSKELSKDTAEKLTKAIDAFAKSFS

SEQ ID NO: 38 - DNA, 555 bp, Unknown
  novel strain
gtggcggaaacgatcgtgtcaggcatggcgggacgctatgcgaccgcgctgttcgagctggcggacgaagccggtgccatcgattccgtccaggcggatcttgatcgcctgtccggccttctggccgagagcgcggatctggcgcggctggtcaagagcccggtcttcaccgccgagcagcagctcggcgcgatggcggccattctcgatcaagcaggcatttccggccttgcgggcaaattcgtgaagctggtggcgcagaaccgccgcctgttcgcactgccgcgcatgattgccgaatacgccgtcctggtggcccggaagaagggcgagacctcggcgagcgtgaccgttgccacccccctgagcgatgagcatctggccacgctcaaggcggccctggctgaaaagaccggcaaggacgtgaagctcgacgtcaccgtcgatccgtccatcctcggtggtctcatcgtgaagctcggctcgcgcatggtcgatgcttccctgaagaccaaactcaattctatccggcatgcgatgaaagaggtccgctga

SEQ ID NO: 39 - protein, 184 aa, Unknown
  novel strain
MAETIVSGMAGRYATALFELADEAGAIDSVQADLDRLSGLLAESADLARLVKSPVFTAEQQLGAMAAILDQAGISGLAGKFVKLVAQNRRLFALPRMIAEYAVLVARKKGETSASVTVATPLSDEHLATLKAALAEKTGKDVKLDVTVDPSILGGLIVKLGSRMVDASLKTKLNSIRHAMKEVR

SEQ ID NO: 40 - DNA, 492 bp, Unknown
  novel strain
atgaccgaaatggaactggctgagctctgggtcgccatcgccttcctggttttcgtaggcctcctgatctatgcgggcgcccaccgcgccatcgtctccgccctggattcccgcggctcgcgcatcgcctcggaactggaggaggcccgtcggctcaaggaagaggcccagaagctggtggccgaattcaagcgcaagcagcgcgaggccgaggccgaggccgaatccatcgtcaccggcgccaaggccgaggccgagcgcctcgccgccgaggccaaggcgaagatcgaggatttcgtcacccgccgcaccaagatggccgaggacaagatcgcccaggccgagcatcaggctctggcggacgtgaagtccatcgccgccgaggcggcggccaaggcggccgaggtgatcctcggcgcccaggccaccggcgcggtggcggagcgtctgctgtcgggcgccatctccgaggtcaagaccaagctcaactga

SEQ ID NO: 41 - protein, 163 aa, Unknown
  novel strain
MTEMELAELWVAIAFLVFVGLLIYAGAHRAIVSALDSRGSRIASELEEARRLKEEAQKLVAEFKRKQREAEAEAESIVTGAKAEAERLAAEAKAKIEDFVTRRTKMAEDKIAQAEHQALADVKSIAAEAAAKAAEVILGAQATGAVAERLLSGAISEVKTKLN

SEQ ID NO: 42 - DNA, 729 bp, Unknown
  novel strain
atgatgattgcatggaagcggaccttcgcagtcgtgaccttcggggccgccctgatggccatgcccgtcgcgggcgtggtcgcagctgagacttctcccgctccggcggcagtggcgcaggccgatcatgcggtgcccaccgaggcggccggccagggcaccgccgatgcggcccatgccgccgcgccgggcgaggccgcccatggtggcgcggccaagcacgaaacccatttcccgcccttcgacggcaccaccttcgcctcccagttgctgtggctcgccgtcaccttcggcctgctttactacctcatgagcaaggtcacgctgccgcgcatcggccgcatcctggaagagcgccacgaccgcatcgccgatgatctggaggaagcctccaagcatcgcgccgagagcgaggccgcccagcgggcctatgagaaggcgctgagcgaggcccgcgcgaaggcccattccatcgccgcggaaacccgcgaccgccttgccgcccacgccgacaccaaccgcaaggcgctggagagcgagctcaccgccaagctgcaggcggccgaggagcgcatcgccaccaccaagagcgaagccctcacccatgtgcgcggcatcgcggtggacgccacccaatccatcgtctccaccctcatcggtgtcgcgcccgcggcggccgacgtggaaaaagcggtggacggcgccctgtcccagcacggccaggcctga

SEQ ID NO: 43 - protein, 242 aa, Unknown
  novel strain
MMIAWKRTFAVVTFGAALMAMPVAGVVAAETSPAPAAVAQADHAVPTEAAGQGTADAAHAAAPGEAAHGGAAKHETHFPPFDGTTFASQLLWLAVTFGLLYYLMSKVTLPRIGRILEERHDRIADDLEEASKHRAESEAAQRAYEKALSEARAKAHSIAAETRDRLAAHADTNRKALESELTAKLQAAEERIATTKSEALTHVRGIAVDATQSIVSTLIGVAPAAADVEKAVDGALSQHGQA

SEQ ID NO: 44 - DNA, 228 bp, Unknown
  novel strain
atggaagcggaagctggaaagttcatcggtgccggcctcgcctgcctcggcatgggtctcgctggcgtcggcgtcggtaacatcttcggtaacttcctctccggcgccctgcgcaacccgtccgctgccgacggccagttcgcccgcgccttcatcggcgccgccctcgcggaaggtctcggcatcttctcgctggtcgttgcgctcgtcctgctgttcgtggcctga

SEQ ID NO: 45 - protein, 75 aa, Unknown
  novel strain
MEAEAGKFIGAGLACLGMGLAGVGVGNIFGNFLSGALRNPSAADGQFARAFIGAALAEGLGIFSLVVALVLLFVA

SEQ ID NO: 46 - DNA, 753 bp, Unknown
  novel strain
atgaccgtcgatccgatccaccagttcgagatcaagcgctacgtggatctgctgaacgtcggcggtgtccagttctccttcaccaacgcaacggtgttcatgattggcatcgtcctggtgattttcttcttcctgactttcgcgacacgcggtcgcacccttgtgccgggccggatgcagtcggcggcggagctgagctacgagttcatcgccaagatggtgcgcgacgcggccggcagcgagggaatggtgttctttcccttcgtcttctcgctcttcatgttcgtgctggtggcgaacgtattggggctcatcccctacaccttcacggtgaccgcccacctcatcgtcaccgccgccctggcggcgacggtgatcctcaccgtcatcatctacggcttcgtgcggcacggcacccacttcctgcacctgttcgtgccgtcgggcgtgccgggcttcctcctgcccttcctcgtggtgatcgaggtggtgtcgttcctgtcgcggcccatcagcctctcgctgcgtctgttcgccaacatgctggcgggccacatcgccctcaaggtgttcgccttcttcgtcgtgggactggcctcggccggcgcgatcggctggttcggcgccaccctgcccttcttcatgatcgtggcgctcaccgcgctggagctgctggtggcggtgctgcaggcctacgtgttcgcggtgctgacctcgatctacctcaacgacgccatccatcccggccactga

SEQ ID NO: 47 - protein, 250 aa, Unknown
  novel strain
MTVDPIHQFEIKRYVDLLNVGGVQFSFTNATVFMIGIVLVIFFFLTFATRGRTLVPGRMQSAAELSYEFIAKMVRDAAGSEGMVFFPFVFSLFMFVLVANVLGLIPYTFTVTAHLIVTAALAATVILTVIIYGFVRHGTHFLHLFVPSGVPGFLLPFLVVIEVVSFLSRPISLSLRLFANMLAGHIALKVFAFFVVGLASAGAIGWFGATLPFFMIVALTALELLVAVLQAYVFAVLTSIYLNDAIHPGH

SEQ ID NO: 48 - DNA, 390 bp, Unknown
  novel strain
atgtccgagccgaatgatccatcccgcagggacggtgcgaaggcgaaagacgagacgcaggactcccggcccggtgaggcggatcttgctcggcgcctcgatgcgctcggcacctccatcggtcaggtcaagtccagaagcggggagcccgcggcgacgccgcgcaaggacacctcctcggcctccggcgcggccctggcgtttcggctgggcgccgagtttgtttcaggcgtgctggtgggctcgctcatcggctacgggttggattatgcgtttgcgatttcgccctgggggctgatcgccttcacgctgatcggctttgccgccggcgtcctgaacatgctgcgcgtggcgaacagcgatgccaagcgccacagcgcggacaggtga

SEQ ID NO: 49 - protein, 129 aa, Unknown
  novel strain
MSEPNDPSRRDGAKAKDETQDSRPGEADLARRLDALGTSIGQVKSRSGEPAATPRKDTSSASGAALAFRLGAEFVSGVLVGSLIGYGLDYAFAISPWGLIAFTLIGFAAGVLNMLRVANSDAKRHSADR

SEQ ID NO: 50 - DNA, 1929 bp, Unknown
  novel strain
atgagttcgctctccgccactattcaacaggtcttcaacgagccgggctgcgcgaagaaccagaataagtccgaggcggagaagaagaagggctgcaccaagcagctgcaacccggcggagcggccggcggctgcgcgttcgacggcgcgaagatcgcgctccagcccttgaccgacgtcgcccacctggtgcacggccccatcgcctgcgaaggcaattcctgggacaatcgtggcgccaagtcctccggctcgaacatctggcgcaccggcttcaccacggacatcaacgaaaccgacgtggtgttcggcggcgagaagcgtctgttcaagtccatcaaggaaatcatcgagaagtacgacccgccggccgtcttcgtctatcagacctgcgtccccgccatgatcggcgacgacatcgacgcggtgtgcaaggcggccagggagaagttcggaaagccggtgatcccgatcaattcccccggcttcgtggggccgaagaatctcggcaacaagctcgccggcgaggcgctcctcgaccatgtgatcggcaccgaggagcccgattacacgacggcctacgacatcaacatcatcggcgaatacaatctctccggcgagttgtggcaggtgaagccgctgctggacgagctgggcatccgcatcctcgcctgcatctccggcgacgggaagtacaaggatgtggcgtcctcccaccgcgccaaggcggcgatgatggtgtgctccaaggccatgatcaacgtggcccgcaagatggaggagcgctacgacatccccttcttcgaaggctccttctacggcatcgaggatagctccgattccctgcgcgagattgcgcgcatgctcatcgagaagggcgccgatccggagctgatggaccgcaccgaggcgctgattgagcgggaagagaagaaggcgtgggacgccatcgccgcctacaagccccgcttcaaggacaagaaggtgctgctcatcaccggcggcgtgaaatcctggtcggtggtggcagcgctccaggaagccggcctcgaactggtgggcacctcggtgaagaagtccaccaaggaggacaaggagcgcatcaaggaactgatgggccaggacgcccacatgatcgacgacatgacgccccgcgaaatgtacaagatgctgaaggacgccaaggcggacatcatgctctcgggcgggcgctcgcaattcatcgcgctcaaggccgccatgccctggctcgacatcaaccaggagcgccaccacgcctatatgggctatgtgggcatggtgaagctggtcgaggagatcgacaaggcgctctacaatcccgtgtgggaacaggtgcgcaagcccgccccgtgggaaaatccggaagacacctggcaggcccgtgcgctcgccgaaatggaggcggaggccgccgcgctcgccgccgatccggtgcgcgcggaagaggtgcgccggtccaagaagatctgcaattgcaagagcgtcgacctcggaaccattgaggacgccatcaaggctcacgcgctgaccaccgtggagggtgtgcgagagcacaccaatgcctcgggaggctgcggagcctgcagcgggcggatcgaggagatcttcgaggccgtgggcgttgtcgccgccccgcctcccgcggaggccgccccgtctccgcaggagatcgcgcccgatccgctcgctgcggaggaaaagcgccgcgccaagaaggcctgcggctgcaaggaggtagcggtcggcaccattgaggatgccatccgcgccaagggtctgcgaaacatcgcggaggtgcgtgcggccaccgatgccaacaccggctgcggcaattgccaggagcgggtggagggcatcctcgaccgggttctcgccgaggcggcctcagaactccaggcggcggaatag

SEQ ID NO: 51 - protein, 642 aa, Unknown
  novel strain
MSSLSATIQQVFNEPGCAKNQNKSEAEKKKGCTKQLQPGGAAGGCAFDGAKIALQPLTDVAHLVHGPIACEGNSWDNRGAKSSGSNIWRTGFTTDINETDVVFGGEKRLFKSIKEIIEKYDPPAVFVYQTCVPAMIGDDIDAVCKAAREKFGKPVIPINSPGFVGPKNLGNKLAGEALLDHVIGTEEPDYTTAYDINIIGEYNLSGELWQVKPLLDELGIRILACISGDGKYKDVASSHRAKAAMMVCSKAMINVARKMEERYDIPFFEGSFYGIEDSSDSLREIARMLIEKGADPELMDRTEALIEREEKKAWDAIAAYKPRFKDKKVLLITGGVKSWSVVAALQEAGLELVGTSVKKSTKEDKERIKELMGQDAHMIDDMTPREMYKMLKDAKADIMLSGGRSQFIALKAAMPWLDINQERHHAYMGYVGMVKLVEEIDKALYNPVWEQVRKPAPWENPEDTWQARALAEMEAEAAALAADPVRAEEVRRSKKICNCKSVDLGTIEDAIKAHALTTVEGVREHTNASGGCGACSGRIEEIFEAVGVVAAPPPAEAAPSPQEIAPDPLAAEEKRRAKKACGCKEVAVGTIEDAIRAKGLRNIAEVRAATDANTGCGNCQERVEGILDRVLAEAASELQAAE

SEQ ID NO: 52 - DNA, 1503 bp, Unknown
  novel strain
atgagtgtcgcacagtcccagagcgtcgccgagatcaaggcgcgcaacaaggaactcatcgaagaggtcctcaaggtctatcccgagaagaccgccaagcgccgcgccaagcacctgaacgtccacgaagccggcaagtccgactgcggcgtgaagtccaacatcaagtccatcccgggcgtgatgaccatccgcggttgcgcttatgccggctccaagggtgtggtgtggggtcccatcaaggacatgatccacatctcccacggcccggtgggctgcggccagtatagctgggccgcccgccgcaactactatatcggcacgaccggcatcgacaccttcgtgacgatgcagttcacctccgacttccaggagaaggacatcgtcttcggcggcgacaagaagctcgccaagatcatggacgagatccaggagctgttcccgctgaacaacggcatcaccgttcagtccgagtgccccatcggcctcatcggcgacgacatcgaggccgtctccaagcagaagtccaaggagtatgagggcaagaccatcgtgccggtgcgctgcgagggcttccgcggcgtgtcccagtccctgggccaccacatcgccaacgacgccatccgcgattgggtgttcgacaagatcgcgcccgacgccgagccgcgctttgagccgaccccgtacgacgtcgccatcatcggcgactacaatatcggtggtgacgcctggtcgtcccgtatcctcctggaggagatgggcctgcgcgtgatcgcccagtggtccggcgacggttcgctcgctgagctggaggccaccccgaaggccaagctcaacgtgctgcactgctaccgctccatgaactacatctcgcgccacatggaagagaagtacggtatcccgtggtgcgagtacaacttcttcggtccttccaagatcgccgagtccctgcgcaagatcgccagctacttcgacgacaagatcaaggaaggcgcggagcgcgtcatcgccaagtatcagccgctcatggatgcggtgatcgcgaagtatcgtccccgcctcgagggcaagaccgtgatgctgtacgtgggcggcctgcgtccccgtcacgtcatcggcgcctacgaggacctgggcatggaagtggtcggcacgggctacgagttcgcccataacgacgactaccagcgcaccgcccagcactacgtcaaggatggcaccatcatctatgacgacgtgaccggctacgagttcgagaagttcgtcgagaagatccagccggacctggtcggttcgggcatcaaggaaaagtacgtcttccagaagatgggcgtgccgttccgccagatgcactcctgggactactcgggcccgtaccacggctatgacggcttcgcgatcttcgcgcgcgacatggacatggccatcaacagccccgtgtggaagatgacccaggctccgtggaagagcgtccccaagccgacgatgctcgcggctgaatga

SEQ ID NO: 53 - protein, 500 aa, Unknown
  novel strain
MSVAQSQSVAEIKARNKELIEEVLKVYPEKTAKRRAKHLNVHEAGKSDCGVKSNIKSIPGVMTIRGCAYAGSKGVVWGPIKDMIHISHGPVGCGQYSWAARRNYYIGTTGIDTFVTMQFTSDFQEKDIVFGGDKKLAKIMDEIQELFPLNNGITVQSECPIGLIGDDIEAVSKQKSKEYEGKTIVPVRCEGFRGVSQSLGHHIANDAIRDWVFDKIAPDAEPRFEPTPYDVAIIGDYNIGGDAWSSRILLEEMGLRVIAQWSGDGSLAELEATPKAKLNVLHCYRSMNYISRHMEEKYGIPWCEYNFFGPSKIAESLRKIASYFDDKIKEGAERVIAKYQPLMDAVIAKYRPRLEGKTVMLYVGGLRPRHVIGAYEDLGMEVVGTGYEFAHNDDYQRTAQHYVKDGTIIYDDVTGYEFEKFVEKIQPDLVGSGIKEKYVFQKMGVPFRQMHSWDYSGPYHGYDGFAIFARDMDMAINSPVWKMTQAPWKSVPKPTMLAAE

SEQ ID NO: 54 - DNA, 1377 bp, Unknown
  novel strain
atggccaccgtttccgtctccaagaaggcctgcgcggtcaaccccctcaagatgagccagccggtgggcggcgcgctcgccttcatgggcgtgcgcaaggccatgccgctgctgcacggctcgcagggctgcacctccttcggcctggtgctgttcgtgcgccacttcaaggaagccatccccatgcagaccaccgccatgagcgaggtggcgacggttctgggcggccttgagaatgtggagcaggccattctcaacatctacaatcgcaccaagccggagatcatcggcatctgctccaccggcgtcaccgagaccaagggcgatgatgtcgacggctacatcaagctgatccgggacaagtatccccagctggccgacttcccgctggtctatgtctccacccccgatttcaaggacgccttccaggacggttgggagaagaccgtggcgaagatggtggaggcgctggtgaagcccgccgccgacaagcagaaggacaagacccgcgtcaacgtcctgcccggctgccacctcacgcccggcgatctggatgagatgcggaccatcttcgaggatttcgggctcacaccctatttcctgccggatctggccggctcgctggatgggcatatccccgaggacttctcgcccaccaccatcggcggcatcggcatcgatgagatcgccaccatgggcgaggcggcccacaccatctgcatcggcgcgcagatgcgccgggcgggcgaggccatggagaagaagaccggcattcccttcaagctgttcgagcgcctgtgcggcctggaggcgaacgacgccttcatcatgcacctgtcgcagatctccggccggccggtgccggtgaagtatcgccggcagcggggccagctggtggatgccatgctggacggccacttccatctgggcggtcgcaaggtggccatgggggcggagccggacctgctctacgacgtgggctccttcctgcacgagatgggcgcccacatcctttccgcggtcaccaccacccagtcgccggtgctggcgcgcctgcctgccgaggaggtgcttatcggcgacctggaggatctggagacccaggcgaaggcgcgcggatgcgatctcctgctcacccattcccatgggcgccaggcggcggagcgcctccacatccccttctaccggatcggcattcccatgtttgaccggctgggggcggggcatctgttgtcggtgggctatcgcggcacccgcgacctcatcttccatctcgccaaccttgtgatcgccgaccacgaggaaaatcacgagccgacgcccgacacctgggccaccggccatggcgagcatgccgccgcccccacttcccattga

SEQ ID NO: 55 - protein, 458 aa, Unknown
  novel strain
MATVSVSKKACAVNPLKMSQPVGGALAFMGVRKAMPLLHGSQGCTSFGLVLFVRHFKEAIPMQTTAMSEVATVLGGLENVEQAILNIYNRTKPEIIGICSTGVTETKGDDVDGYIKLIRDKYPQLADFPLVYVSTPDFKDAFQDGWEKTVAKMVEALVKPAADKQKDKTRVNVLPGCHLTPGDLDEMRTIFEDFGLTPYFLPDLAGSLDGHIPEDFSPTTIGGIGIDEIATMGEAAHTICIGAQMRRAGEAMEKKTGIPFKLFERLCGLEANDAFIMHLSQISGRPVPVKYRRQRGQLVDAMLDGHFHLGGRKVAMGAEPDLLYDVGSFLHEMGAHILSAVTTTQSPVLARLPAEEVLIGDLEDLETQAKARGCDLLLTHSHGRQAAERLHIPFYRIGIPMFDRLGAGHLLSVGYRGTRDLIFHLANLVIADHEENHEPTPDTWATGHGEHAAAPTSH

SEQ ID NO: 56 - DNA, 1560 bp, Unknown
  novel strain
atgccacaaaatgctgacaatgtgctcgatcacttcgagctcttccgtggtcccgaataccagcagatgctggccaataagaaaaagatgttcgagaacccccgcgatccggccgaagtcgagcgcgtgcgggaatgggcgaagactcctgaatacaaggagctgaacttcgcccgcgaggcgctcaccgtgaatccggccaaggcttgtcagccgctgggcgcggtgttcgtcgccgtcggcttcgagagcacgatccccttcgtgcacggctcgcagggttgcgtcgcgtattaccgctcgcacctctcccgccacttcaaggagccgtcctcctgcgtctcctcgtccatgaccgaggatgcggcggtgttcggcggcctcaacaacatgattgacggcctcgccaacacctacaacatgtacaagccgaagatgatcgccgtctccaccacctgcatggcggaagtcatcggcgacgatctgaacgccttcatcaagaccgcgaaggaaaagggctcggttccggccgaatacgacgtgcccttcgcccacaccccggcgttcgtcggcagccatgtcaccggctacgacaatgcgctcaagggcatcctcgagcacttctgggacggcaaggccggcaccgcgccgaagctggagcgcgttcccaacgagaagatcaacttcatcggcggcttcgacggctacaccgtcggcaacactcgcgaagtgaagcgcatcttcgaggcgttcggcgccgattacaccatcctcgccgacaattccgaagtgttcgacaccccgaccgacggcgagttccgcatgtatgacggcggcacgaccctggaggacgcggcgaacgcggtgcacgccaaggccaccatctccatgcaggaatactgcacggagaagaccctgcccatgatcgccggtcatggccaggacgtggtcgccctcaaccaccccgtgggcgtgggcggcaccgacaagttcctcatggagatcgcccgcctcaccggcaaggagatccccgaggagctgacccgcgagcgcggccgtctcgtggacgctatcgcggactcttccgcgcacatccacggcaagaagttcgccatctacggcgatccggatctgtgcctgggcctcgccgcgttcctgctggagctgggcgccgagccgacccatgtgctggccaccaacggcaccaagaagtgggccgagaaggttcaggaactgttcgactcttcgccgttcggcgccaactgcaaggtctatcccggcaaggacctgtggcacatgcgctcgctcctgttcgtggagccggtggatttcatcatcggcaacacctacggcaagtatctcgagcgcgacacgggcaccccgctgatccgtatcggcttcccggtgttcgaccgtcaccaccaccaccgccgtccggtgtggggctatcagggcggcatgaacgtcctgatcacgatcctcgacaagatctttgacgagatcgaccgcaacaccaacgtgccggccaagaccgactactcgttcgacatcattcgttga

SEQ ID NO: 57 - protein, 519 aa, Unknown
  novel strain
MPQNADNVLDHFELFRGPEYQQMLANKKKMFENPRDPAEVERVREWAKTPEYKELNFAREALTVNPAKACQPLGAVFVAVGFESTIPFVHGSQGCVAYYRSHLSRHFKEPSSCVSSSMTEDAAVFGGLNNMIDGLANTYNMYKPKMIAVSTTCMAEVIGDDLNAFIKTAKEKGSVPAEYDVPFAHTPAFVGSHVTGYDNALKGILEHFWDGKAGTAPKLERVPNEKINFIGGFDGYTVGNTREVKRIFEAFGADYTILADNSEVFDTPTDGEFRMYDGGTTLEDAANAVHAKATISMQEYCTEKTLPMIAGHGQDVVALNHPVGVGGTDKFLMEIARLTGKEIPEELTRERGRLVDAIADSSAHIHGKKFAIYGDPDLCLGLAAFLLELGAEPTHVLATNGTKKWAEKVQELFDSSPFGANCKVYPGKDLWHMRSLLFVEPVDFIIGNTYGKYLERDTGTPLIRIGFPVFDRHHHHRRPVWGYQGGMNVLITILDKIFDEIDRNTNVPAKTDYSFDIIR

SEQ ID NO: 58 - DNA, 621 bp, Unknown
  novel strain
gtggagtccggtggtcctgagccgggcgtgggctgcgccggccgcggcgtgatcacctccatcaacttcctggaggagaacggcgcctacgaggacatcgactatgtgtcctacgacgtgctgggcgacgtggtgtgcggcggcttcgccatgcccatccgcgagaacaaggcgcaggaaatctacatcgtgatgtccggcgagatgatggccatgtatgcggccaacaacatctccaagggcatcctgaagtatgccaattccggcggcgtgcgcctgggcgggctggtctgcaacgagcgccagaccgacaaggagctggagctggcggaggctctggcgaagaagctcggcaccgagctgatctacttcgtgccgcgcgacaacatcgtgcagcatgccgagctgcgccgcatgacagtgatcgagtatgcgcccgattccgcccaggcccagcactaccggaacctggccgagaaggtgcacgccaacaagggcaacggcatcatcccgaccccgatcaccatggacgagctggaagacatgctcatggagcacggcatcatgaaggccgtggacgagagccagatcggcaagaccgccgccgagctcgccgtctga

SEQ ID NO: 59 - protein, 206 aa, unknown
  novel strain
MESGGPEPGVGCAGRGVITSINFLEENGAYEDIDYVSYDVLGDVVCGGFAMPIRENKAQEIYIVMSGEMMAMYAANNISKGILKYANSGGVRLGGLVCNERQTDKELELAEALAKKLGTELIYFVPRDNIVQHAELRRMTVIEYAPDSAQAQHYRNLAEKVHANKGNGIIPTPITMDELEDMLMEHGIMKAVDESQIGKTAAELAV

SEQ ID NO: 60 - DNA, 2646 bp, unknown
  novel strain
atgtccgcagccgaggaaacgtccacgcacgctgaattgaggctcccgcaggatggggtggagcatgacgtcgcggccgccgaggccgcggtggaccggtccgccggggagccgtcgggaacgcaggcgtccgccgcgcccgccgaggcggcgccgtcttccgctcctgtttccgctgctgccggagaaacgcagccacaggacgataccccgccgcaggacccgtcttccctgtcggacggtccaggcctgtcgccgcccgtgacccagcccggcgcggcgtccggcgacttcggcgggcccgaggccatgggcgacgccttcatgccgcctgtgccggaagagcccatggaggggatggcccccgcgccggcaatctccgcggcgcccgcctcggtgccgtctgccggctggccggaggatgcgccttcggcactgctcgatgcggtggatgcgtccggggacctgccggctgccgcggagggcgcggccacggcgccggagcccattttccgcgagctgcccatgacggcggcgcccgccgcacctgccatcgcgaacattctcgagccggtggccgaggctctgtccgcgctcggggcggtggccgtctcccggccccaggtccagcgcgaattccgcgcggcgcccgaacacccccccatgcccaggatggcgccccctcaggcggcgccagaaccggcccctgccgttccgcccaaacctgaagctgccaaacctgaagctgccaaacctgaagccgccaagcctgaagctgccaagcctgaagttgccaagccggacgccgccgcgccggacgccgcgaaatcttcgggcaaggcggagcgtccgtccggcgccggggacggctctggaacgtcgggcgtgaacatggaggccttctcccgcaacctcgcccgtctggtggaagagggcggcaaggccatggccgcctatctgagcccacgggagcagggcaagaccgacgatctcgccgatgacatcgccgatgccatgaagaccgtgggccaggtggtggaatactgggtcgccgatccccagcgcacggtggaggcgcagtcccgcctcatgggcggctatctctcggtctgggccaacaccctgaagcgcctggcaggcgaggaggccacgccggtggccgcccccgacccgaaggatgcccgcttcaaggatgccggctggaacgacagccccatgttcgatgccctcaagcaggcctatctggtcacctcggactgggcgcagaacatggtggacgaggccaagggcctcgatccccacaccaagcacaaggcggagttcctggtgcggcagatcgccaacgccatctcgccctccaacttcgtcctcaccaatcccgagctcatccgcgagaccctgcactcgtcgggcgagaatctggtgaagggcatgcagaacctcaccgcggatctgatggcggggcagggcacgctgaagatccgccagacggatctttccgccttcgaggtgggccgcaacctcgccaccacgccgggcaaggtgatcttcgaaaacgagctgatgcagctcatccagtacgagccgaccaccgagacggtgaagaagacgccggtgctcatcgtcccgccctggatcaacaagttctacatcctcgacctgacggcggaaaaatccttgatcaagtggctggtgagccagggactgacggtcttcaccatctcctgggtcaatcccgacgggcgcctcgccgccaagggcttcgacgattacatgcgcgacggcatcatggccgccctcgacgcggtggcggtggcctccggcgagcggcgggcccatgcggtgggctattgcgtgggcggcacgctgctggccaccacgctcgcttacatggccgccaccggggatgaccgcatcgccagcgccaccttcctcaccacccagatcgacttcacccatgcgggcgatctgaaggtgttcgtggacgaaagccagctcgccaccatcgagcgcaagatgaaggagatggggtatctggaaggctcgaagatggcctccgccttcaacatgctgcgctccaacgacctgatctggccctatgtggtgaacaactacatgaagggcaaggcgccgtttccgttcgacctgctgttctggaattcggattccacccgcatgccggcggcgaaccattcctattatctgcgcaactgctacctcaccaacaatatcgcccggggtctggcggagctggccggcctcaagatcgatgtcaccaaggtgtcgatcccagtctattcgctggcgacgcgggaagaccacatcgccccggccaactcggtctatatcggggcaaatctcctgtccggcccggtgcgctacgtgctggcgggctcgggccacatcgccggggtggtgaacccgccggccaagatgaagtatcagtactgggccgacggcccggtggggccgagctacgaggcctggctggccggggcgcaggagcacaagggctcctggtggccggactggttcaactggttctccttcaaccatcccgaggaggtgccggcgcgcgccatcggcggcggccgcctcgcccccatcgaggacgcacccgggcgctatgtgaaggagcggtcgtag

SEQ ID NO: 61 - DNA, 1950 bp, unknown
  novel strain
atggaagcgcgaaagatgcccgtttcacccccctcctccgccaccatcctgcccctgcccgtctcggccgctcccccttcgacggcgcccgccgcgtccctgccggcgacggcctccagctccaccaacaaggccagcccctcagccttccccgccgcctgggcgcgttccttcgccctgcccggcctgcccgccttcctgtgcccggacgaggaggatttcgaggagggttcggggccggccgccttccatgcggtggaccgggcggcggcggccttggtggcccgcaccacccaggggctctcgcccgccgccctcaccctcgcttacatggattgggcgatgcatctggcagcggcgccgggcaagcaggcggagctggcggtgaaggccacgcgcaaggcggcgcgcttctgggcctatgtgctcgcctccaccctcgaccgcacccaggcgccctgcatcgcgcccctggtgggcgacgaacggttttccgccccggcctggcaggactggccctaccgcttctggtaccaggcctttctgctcaatcagcaatggtggcacaacgccactcatggcgtgccgggcgtggcgccgcacaatcaggacgtggtggccttcgccgcccggcaggtcctggacatgttctccccctccaactcgcccctcaccaatcccgaggtggtgaagaaggcgcgccagacgctaggggccaatttcgtccagggcgcgcgcaacttcatggaggaccagtcgcgcaaaaccaccggccgcccgcccgtgggcgcggaggcgttcactcccggcaaggaggtggccatcacccccggcgaggtgatctaccgcaaccatctcatcgagctgatccaataccgggcgaccacccctgacgtgcatgcggagccgatcctcatcgtgcccgcctggatcatgaaatattacatcctcgacctgtcgcccgataactccctgatccgctacctggtggacaaggggcacacggtcttctgcatctcctggcgcaatgtgaacgcggaggaccgcgatctcggcttcgaggactatcgcaagatgggcatcatggcggccctcgacgcggtgaacgcggtggtgccgaaccagaaggtgcatgcggtgggttattgcctgggcggcacgctgctctccatcgccgccgccgccatggcgcgggtggtcgacgaccggctgggctccgtcaccctgttcgccgcccagaccgacttcaccgagcccggcgaactgcagctcttcgtggacccgagcgagctttatgccctggaaagcctcatgtgggaccagggctatctcggcgcccggcagatggccggcgcgttcgagatgctgcgctccaacgacctcgtctggtcgcgcatggtgcgcgactatctcatgggcgagcgcgcacccatgaacgacctcatggcctggaacgcagatgccacccgcatgccctatcgcatgcattcccagtatctgcgcaacctgttcctcgacaacgagctggccgtgggccgttacatggtggagggccggccggtgtcgctgcagaacatccgcgtcccgctgttcgtggtgggcacggagcgggaccacgtggcgccgtggaagtcggtctacaagatccaccagctcaccgacacggacgtgaccttcgtcctcgcctccggcgggcacaatgcgggcatcgtctccgagcccggccacaagcaccggcactatcgcatccacgacaccaaattgggcgagatgcatgtgagcccggaggaatggatggaggcgaaccggtcgcaggacgggtcctggtggccggcctgggaggcatggctcgccggccagtcctccggccgcatcggcctgccgcctctgggcgcgcccggctacgaggtgctgggcccggcgcccggcacctatgtgatgcaaaggtga

SEQ ID NO: 62 - protein, 881 aa, unknown
  novel strain
MSAAEETSTHAELRLPQDGVEHDVAAAEAAVDRSAGEPSGTQASAAPAEAAPSSAPVSAAAGETQPQDDTPPQDPSSLSDGPGLSPPVTQPGAASGDFGGPEAMGDAFMPPVPEEPMEGMAPAPAISAAPASVPSAGWPEDAPSALLDAVDASGDLPAAAEGAATAPEPIFRELPMTAAPAAPAIANILEPVAEALSALGAVAVSRPQVQREFRAAPEHPPMPRMAPPQAAPEPAPAVPPKPEAAKPEAAKPEAAKPEAAKPEVAKPDAAAPDAAKSSGKAERPSGAGDGSGTSGVNMEAFSRNLARLVEEGGKAMAAYLSPREQGKTDDLADDIADAMKTVGQVVEYWVADPQRTVEAQSRLMGGYLSVWANTLKRLAGEEATPVAAPDPKDARFKDAGWNDSPMFDALKQAYLVTSDWAQNMVDEAKGLDPHTKHKAEFLVRQIANAISPSNFVLTNPELIRETLHSSGENLVKGMQNLTADLMAGQGTLKIRQTDLSAFEVGRNLATTPGKVIFENELMQLIQYEPTTETVKKTPVLIVPPWINKFYILDLTAEKSLIKWLVSQGLTVFTISWVNPDGRLAAKGFDDYMRDGIMAALDAVAVASGERRAHAVGYCVGGTLLATTLAYMAATGDDRIASATFLTTQIDFTHAGDLKVFVDESQLATIERKMKEMGYLEGSKMASAFNMLRSNDLIWPYVVNNYMKGKAPFPFDLLFWNSDSTRMPAANHSYYLRNCYLTNNIARGLAELAGLKIDVTKVSIPVYSLATREDHIAPANSVYIGANLLSGPVRYVLAGSGHIAGVVNPPAKMKYQYWADGPVGPSYEAWLAGAQEHKGSWWPDWFNWFSFNHPEEVPARAIGGGRLAPIEDAPGRYVKERS

SEQ ID NO: 63 - protein, 649 aa, unknown
  novel strain
MEARKMPVSPPSSATILPLPVSAAPPSTAPAASLPATASSSTNKASPSAFPAAWARSFALPGLPAFLCPDEEDFEEGSGPAAFHAVDRAAAALVARTTQGLSPAALTLAYMDWAMHLAAAPGKQAELAVKATRKAARFWAYVLASTLDRTQAPCIAPLVGDERFSAPAWQDWPYRFWYQAFLLNQQWWHNATHGVPGVAPHNQDVVAFAARQVLDMFSPSNSPLTNPEVVKKARQTLGANFVQGARNFMEDQSRKTTGRPPVGAEAFTPGKEVAITPGEVIYRNHLIELIQYRATTPDVHAEPILIVPAWIMKYYILDLSPDNSLIRYLVDKGHTVFCISWRNVNAEDRDLGFEDYRKMGIMAALDAVNAVVPNQKVHAVGYCLGGTLLSIAAAAMARVVDDRLGSVTLFAAQTDFTEPGELQLFVDPSELYALESLMWDQGYLGARQMAGAFEMLRSNDLVWSRMVRDYLMGERAPMNDLMAWNADATRMPYRMHSQYLRNLFLDNELAVGRYMVEGRPVSLQNIRVPLFVVGTERDHVAPWKSVYKIHQLTDTDVTFVLASGGHNAGIVSEPGHKHRHYRIHDTKLGEMHVSPEEWMEANRSQDGSWWPAWEAWLAGQSSGRIGLPPLGAPGYEVLGPAPGTYVMQR

(19) United States
(12) Patent Application Publication
(10) Pub. No.: US 2024/0254435 A1
(43) Pub. Date: Aug. 1, 2024

(54) VARIANT BACTERIAL STRAINS AND PROCESSES FOR PROTEIN OR BIOMASS PRODUCTION

(71) Applicant: Solar Foods Oy, Lappeenranta (FI)

(72) Inventors: Tytti JÄMSÄ, Lappeenranta (FI); Juha-Pekka PITKÄNEN, Lappeenranta (FI); Laura SALUSJÄRVI, Espoo (FI)

(73) Assignee: Solar Foods Oy, Lappeenranta (FI)

(21) Appl. No.: 18/553,946

(22) PCT Filed: Apr. 7, 2022
(86) PCT No.: PCT/FI2022/050229

§ 371 Date: Oct. 4, 2023

(30) Foreign Application Priority Data
Apr. 28, 2021 (FI) 20215494

(51) Int. Cl. C12N 1/20; C12N 9/10; C12N 15/90; C12R 1/01

(52) U.S. Cl. CPC C12N 1/205; C12N 1/20; C12N 9/1029; C12N 15/902; C12Y 203/01; C12R 2001/01

(57) ABSTRACT

Variant chemoautotrophic bacteria of the genus Xanthobacter including a genetic modification that reduces the bacterial production of polyhydroxyalkanoic acids. Furthermore, the present disclosure relates to continuous culture processes for the production of protein or biomass using variant chemoautotrophic bacteria, said process including supply of gases and minerals to the cells. The present disclosure also relates to the products of these processes and use of these products in e.g. food or feed.

Reference is made to the Identification of the Microorganism, having the Identification reference given by the DEPOSITOR of SoF1-2.0 and with the Accession number given by the INTERNATIONAL DEPOSITORY AUTHORITY of VTT E-213595. The date of the original deposit is Apr. 19, 2021

### INCORPORATION BY REFERENCE

The sequence listing in text (.txt) format is incorporated herein by reference in its entirety.
a. Name of File: Solar015PCT Sequence Listing_ST25
b. Date of Creation: 1 Mar. 2024
c. Size of File: 147,363 Bytes

### FIELD

The aspects of the disclosed embodiments relate to the production of protein and/or other macromolecules using microorganisms. In particular, the aspects of the disclosed embodiments relate to novel bacterial strains and continuous culture processes for the production of protein or biomass using bacteria wherein gases and minerals are supplied to the cells. The aspects of the disclosed embodiments also relate to the products of these processes and use of these products in e.g., food or feed.

### BACKGROUND

Growing world population, climate change and shortage of water increasingly pose a threat to traditional agriculture and thus sufficient supply of food and feed. Therefore, alternative sources of organic molecules, such as proteins, are being investigated. A potential alternative is single cell production, i.e. the production of protein and/or other macromolecules using microorganisms.

Chemoautotrophic microorganisms have been described which are able to grow on minimal mineral medium with hydrogen gas as the energy source and carbon dioxide as the only carbon source. For a review of these microorganisms, see e.g. Shively et al. (1998) Annu Rev Microbiol 52:191. Patent application WO2018144965 describes various microorganisms and bioprocesses for converting gaseous substrates into high-protein biomass. Andersen et al. (1979) Biochim Biophys Acta 585:1-11 describes mutant strains of Alcaligenes eutrophus, a hydrogen bacterium that grows readily under heterotrophic and autotrophic conditions. Mutants having altered ribulose-1,5-bisphosphate carboxylase/oxygenase (rubisco) activity were characterised. Ohmiya et al. (2003) J. Biosci. Bioeng. 95:549-561 reviews the application of microbial genes to recalcitrant biomass utilization. Yu Jian et al. (2013) Int J Hydrogen Ener 38:8683-8690 describes carbon dioxide fixation by a hydrogen-oxidizing bacterial isolate. A high energy efficiency of 50% was measured under a moderate oxygen concentration (10 mol %).

However, various chemoautotrophic microorganisms have different properties in terms of growth rate, yield, biomass composition as well as properties related to being used as a food ingredient such as safety in human consumption, taste, smell, mouth-feel, technical and functional properties in cooking, etc. Not every chemoautotrophic microorganism has sufficient growth rate and provides sufficient yield and not every process can realistically be upscaled to an economically viable large-scale process. In order to have sufficient output of functional protein, e.g. for food or feed applications, it is important to find a suitable production organism and a suitable process which can be performed at large scale. This need is addressed by the present invention.

Under metabolic stress, such as nitrogen limitation, bacteria may store energy in the form of storage polymers called polyhydroxyalkanoic acids (PHAs) (Rehm and Steinbüchel, 1999 Int J Biol Macromol 25:3-19). When bacteria are grown in a large bioreactor in an industrial setting, gas exchange often becomes insufficient and leads to increased PHA production. PHAs can be utilized as bioplastics, but when the bacteria are grown for other products than PHAs, its production is an unwanted outcome as it reduces the carbon yield. This problem is also address by the present invention.

### SUMMARY

The inventors found that chemoautotrophic bacteria, for example of the genus Xanthobacter, under certain conditions store energy in the form of PHAs. It was found that variant strains comprising a gene disruption of the phaC1 gene produced almost no PHA under the same conditions, but retained favourable properties and suitability for processes for the production of biomass and/or protein.

In a first main aspect, the aspects of the disclosed embodiments relate to a variant of bacterial strain VTT-E-193585 comprising a genetic modification that reduces the bacterial production of polyhydroxyalkanoic acid (PHA) as compared to strain VTT-E-193585.

In a further aspect, aspects of the disclosed embodiments relate to a process for the production of biomass and/or protein, said process comprising culturing a variant chemoautotrophic bacterial strain in continuous culture with hydrogen as energy source and an inorganic carbon source, wherein the inorganic carbon source comprises carbon dioxide and wherein said variant chemoautotrophic strain comprises a gene disruption of one or more genes encoding a PHA synthase. In further main aspects, the invention relates to bulk protein, biomass or non-protein cellular or chemical components obtained or obtainable by the process of the invention, and to a food or feed product obtained or obtainable by a process of the invention.

In a further aspect, the aspects of the disclosed embodiments relate to a variant Xanthobacter strain comprising a gene disruption of one or more genes encoding a PHA synthase.

The aspects of the disclosed embodiments also relate to methods for genetic modification of bacterial strain VTT-E-193585 and to genetically-modified variants of strain VTT-E-193585.

### BRIEF DESCRIPTION OF THE FIGURES

FIG. 1. Optical density measured at 600 nm (black circles) and optical density probe readings during chemoautotrophic 200-L cultivation of isolated bacterial strain deposited as VTT-E-193585.

FIG. 2. Optical density measured at 600 nm during parallel chemoautotrophic 200-mL cultivations of isolated bacterial strain deposited as VTT-E-193585 on different nitrogen sources.

FIG. 3. Growth curve of SoF1 (VTT-E-193585) (solid line) and SoF1-2.0 (VTT E-213595) (dashed line) in autotrophic conditions.

### DETAILED DESCRIPTION

### Definitions

When used herein, the term “isolated”, e.g. in the context of a strain, means isolated from its natural environment. Preferably, an isolated strain is pure, i.e. free of other strains.

The term “variant”, when used herein in the context of a strain, refers to a strain which is derived from a reference strain, i.e. generated using the reference strain as starting point, and contains a genetic modification as compared to said reference strain. Genetic modifications include modifications include point mutations, as well as disruptions, such as insertions or deletions, of entire loci or fragments thereof. The variant preferably has fewer than 10 genetic modifications, e.g. fewer than 5, such as 4, 3, 2 or 1 genetic modification(s) compared to the reference strain. Preferably, the genome sequence of the variant strain is more than 90%, such as more than 95%, for example more than 99% identical to the genome sequence of the reference strain.

The term “chemoautotrophic” when used herein, refers to the ability to grow on minimal mineral medium with hydrogen gas as the energy source and carbon dioxide as the only carbon source.

When used herein, the noun “culture” refers to a suspension of viable cells in a liquid medium.

The term “biomass” has its usual meaning in the field of bacterial fermentation and refers to cellular material.

The term “continuous culture”, when used herein, refers to a culturing process wherein fresh media is added continuously to the culture and media with bacterial culture is removed continuously at essentially the same rate.

### Aspects and Embodiments

Strain VTT-E-193585 has been isolated from the seashore of the Baltic sea in Naantali, Finland. This organism is able to grow in suitable bioreactor conditions with minimal mineral medium with hydrogen as the energy source and carbon dioxide as the carbon source at limited oxygen conditions. 16S sequencing and Illumina metagenomics sequencing have shown that the strain most likely is a member of the genus Xanthobacter, but is not a known species. The bacterial strain is highly suitable for food and feed applications, because the dried cell powder has a high protein content and contains all the essential amino acids. It also contains more unsaturated than saturated fatty acids and a high level of B-group vitamins. The levels of peptidoglycans and lipopolysaccharides, which may cause allergy or toxicity, are low. A toxicity analysis was performed and no genotoxicity or cytotoxicity was observed for the strain. In addition, the strain is generally sensitive to antibiotics.

Strain VTT-E-193585 (SoF1) has been deposited on Jun. 11, 2019 in the VTT Culture Collection at the VTT Technical Research Centre of Finland, P.O. Box 1000, FI-02044 VTT, Finland, an International Depositary Authority under the Budapest Treaty. Further information on the characteristics of the strain and methods for culturing the strain are provided in the Examples herein and in European patent application EP19205786.7 (incorporated herein by reference).

The aspects of the disclosed embodiments relate inter alia to variants of VTT-E-193585, in particular variants comprising a genetic modification that reduces the bacterial production of polyhydroxyalkanoic acid (PHA), as well as more generally to chemoautotrophic bacteria having reduced production of polyhydroxyalkanoic acid (PHA), such as strains comprising a gene disruption of one or more genes encoding a PHA synthase.

The inventors have constructed genetically-modified variants of VTT-E-193585 comprising disruptions of the phaC1 and/or phaC2 loci. The variant comprising a gene disruption of phaC1 has been deposited on Apr. 19, 2021 in the VTT Culture Collection at the VTT Technical Research Centre of Finland, P.O. Box 1000, FI-02044 VTT, Finland, an International Depositary Authority under the Budapest Treaty. The accession number is VTT E-213595. Further information on the characteristics of the strain and methods for culturing the strain are provided in the Examples herein.

In a first main aspect, the aspects of the disclosed embodiments relate to a variant of bacterial strain VTT-E-193585 comprising a genetic modification that reduces the bacterial production of polyhydroxyalkanoic acid (PHA) as compared to strain VTT-E-193585. Thus, the invention relates to a genetically-modified variant, i.e. derivative, of bacterial strain VTT-E-193585. In other words, strain VTT-E-193585 further characterized in that it comprises a genetic modification.

In one embodiment, the genetic modification reduces bacterial PHA synthase activity as compared to strain VTT-E-193585, preferably wherein PHA synthase activity has been reduced to less than 10%, such as less than 5%, for example less than 2%.

In a further embodiment, the genetic modification reduces bacterial PHB production under autotrophic growth conditions to less than 10%, such as less than 5%, for example less than 2%. This can e.g. be determined by measuring the PHB dry content as described in Example 5 herein.

In one embodiment, the variant comprises a genetic modification reducing the expression level of phaC1 and/or the activity of the phaC1 enzyme.

In another embodiment, the variant comprises a genetic modification reducing the expression level of phaC2 and/or the activity of the phaC2 enzyme.

In one embodiment, the genetic modification is a gene disruption, such as an insertion and/or a deletion of the gene or part thereof.

In one embodiment, the variant comprises gene disruptions of both phaC1 and phaC2.

In another embodiment, the variant comprises a gene disruption of phaC1 but not of phaC2.

In one embodiment, the variant is the bacterial strain deposited under number VTT-E-213595, in which the phaC1 gene has been disrupted.

In a preferred embodiment, the variant has retained the ability to grow using hydrogen gas as energy source and carbon dioxide as the only carbon source.

In one embodiment, if the strain is a variant of strain VTT-E-193585, the variant comprises the 16S ribosomal RNA set forth in SEQ ID NO:1 or a 16S ribosomal RNA having up to 20 nucleotide differences with SEQ ID NO:1, e.g. 1 to 10, such as 1 to 5, e.g. one, two or three nucleotide differences with SEQ ID NO:1.

SEQ ID NO: 1. 16S ribosomal RNA sequence of strain VTT-E-193585:
CTTGAGAGTTTGATCCTGGCTCAGAGCGAACGCTGGCGGCAGGCCTAACACATGCAAGTCGA
GCGCCCAGCAATGGGAGCGGCAGACGGGTGAGTAACGCGTGGGGATGTGCCCAATGGTACG
GAATAACCCAGGGAAACTTGGACTAATACCGTATGAGCCCTTCGGGGGAAAGATTTATCGCCA
TTGGATCAACCCGCGTCTGATTAGCTAGTTGGTGGGGTAACGGCCCACCAAGGCGACGATCA
GTAGCTGGTCTGAGAGGATGATCAGCCACACTGGGACTGAGACACGGCCCAGACTCCTACGG
GAGGCAGCAGTGGGGAATATTGGACAATGGGCGCAAGCCTGATCCAGCCATGCCGCGTGTG
TGATGAAGGCCTTAGGGTTGTAAAGCACTTTCGCCGGTGAAGATAATGACGGTAACCGGAGA
AGAAGCCCCGGCTAACTTCGTGCCAGCAGCCGCGGTAATACGAAGGGGGCTAGCGTTGCTCG
GAATCACTGGGCGTAAAGCGCACGTAGGCGGATCGTTAAGTCAGGGGTGAAATCCTGGAGCT
CAACTCCAGAACTGCCCTTGATACTGGCGACCTTGAGTTCGAGAGAGGTTGGTGGAACTGCG
AGTGTAGAGGTGAAATTCGTAGATATTCGCAAGAACACCAGTGGCGAAGGCGGCCAACTGGC
TCGATACTGACGCTGAGGTGCGAAAGCGTGGGGAGCAAACAGGATTAGATACCCTGGTAGTC
CACGCCGTAAACGATGGATGCTAGCCGTTGGGCAGCTTGCTGTTCAGTGGCGCAGCTAACGC
ATTAAGCATCCCGCCTGGGGAGTACGGTCGCAAGATTAAAACTCAAAGGAATTGACGGGGGC
CCGCACAAGCGGTGGAGCATGTGGTTTAATTCGAAGCAACGCGCAGAACCTTACCAGCCTTT
GACATGGCAGGACGATTTCCAGAGATGGATCTCTTCCAGCAATGGACCTGCACACAGGTGCT
GCATGGCTGTCGTCAGCTCGTGTCGTGAGATGTTGGGTTAAGTCCCGCAACGAGCGCAACCC
TCGCCTCTAGTTGCCAGCATTCAGTTGGGCACTCTAGAGGGACTGCCGGTGATAAGCCGAGA
GGAAGGTGGGGATGACGTCAAGTCCTCATGGCCCTTACGGGCTGGGCTACACACGTGCTACA
ATGGTGGTGACAGTGGGATGCGAAAGGGCGACCTCTAGCAAATCTCCAAAAGCCATCTCAGT
TCGGATTGTACTCTGCAACTCGAGTGCATGAAGTTGGAATCGCTAGTAATCGTGGATCAGCAT
GCCACGGTGAATACGTTCCCGGGCCTTGTACACACCGCCCGTCACACCATGGGAGTTGGCTT
TACCCGAAGGCGCTGCGCTAACCCGCAAGGGAGGCAGGCGACCACGGTAGGGTCAGCGACT
GGGGTGAAGTCGTAACAAGGTAGCCGTAGGGGAACCTGCGGCTGGATCACCTCCTTT

In a further aspect, the aspects of the disclosed embodiments relate to a variant Xanthobacter strain comprising a gene disruption of one or more genes encoding a PHA synthase. Preferably, the strain is selected from the group consisting of: X. agilis, X. aminoxidans, X. autotrophicus, X. flavus, X. tagetidis, X. viscosus, Xanthobacter sp. 126, Xanthobacter sp. 91 and strain VTT-E-193585.

In one embodiment, the variant Xanthobacter strain is a variant X. autotrophicus strain wherein the phaC gene (UniProtKB—A0A6C1KXK2) has been disrupted.

In another embodiment, the variant Xanthobacter strain is a variant X. tagetidis strain wherein the phaC gene (UniProtKB—A0A3L7AJD5) has been disrupted.

In another embodiment, the variant Xanthobacter strain is a variant of a Xanthobacter species wherein the gene in the genome of said strain that has the highest sequence identity to phaC1 (SEQ ID NO: 62) has been disrupted. Methods for determining sequence identity are well-known in the art. Preferably, the encoded protein has more than 50% sequence identity, such as more than 60%, for example more than 70%, such as more than 80%, for example more than 90%, such as more than 95% sequence identity to SEQ ID NO: 62.

In another embodiment, the variant Xanthobacter strain is a variant of strain X. agilis, X. aminoxidans, X. flavus, X. viscosus, Xanthobacter sp. 126 or Xanthobacter sp. 91 wherein the gene in the genome of said strain that encodes the protein that has the highest sequence identity to phaC1 (SEQ ID NO:62) has been disrupted. For example, in one embodiment, the variant is a variant of X. agilis wherein the gene of X. agilis, that has the highest sequence identity to phaC1 (SEQ ID NO:62) of all genes in the X. agilis genome has been disrupted.

In one embodiment, the variant Xanthobacter strain is a variant X. tagetidis strain wherein the gene set forth in NCBI ref. MBB6309058.1 has been disrupted.

In one embodiment, the variant Xanthobacter strain is a variant X. flavus strain wherein the gene set forth in NCBI ref. MBP2147722.1 has been disrupted.

In one embodiment, the variant Xanthobacter strain is a variant X. autotrophicus wherein the gene set forth in NCBI ref. WP_138398147.1 has been disrupted.

In one embodiment, the variant Xanthobacter strain is a variant X. tagetidis strain wherein the gene set forth in NCBI ref. WP_210210858.1has been disrupted.

In one embodiment, the variant Xanthobacter strain is a variant X. flavus strain wherein the gene set forth in NCBI ref. WP_209489961.1 has been disrupted.

In one embodiment, the variant Xanthobacter strain is a variant X. autotrophicus Py2 strain wherein the gene set forth in NCBI ref. ABS67253.1 has been disrupted.

In a further aspect, the aspects of the disclosed embodiments relate to a culture comprising the variant bacterial strain of the invention. In a preferred embodiment, the volume of the culture is 100 ml or more, e.g. 1 L or more, such as 10 L or more, e.g. 1,000 L or more, such as 10,000 L or more, e.g. 50,000 L or more, such as 100,000 L or more, e.g. 200,000 L or more.

In a further aspect, the aspects of the disclosed embodiments relate to a process for the production of biomass and/or protein, said process comprising culturing the variant bacterial strain of the invention. In one embodiment, the process is for the production of biomass. In another embodiment, the process is for the production of protein. In one embodiment, the process comprises culturing the bacteria in continuous culture with hydrogen as energy source and an inorganic carbon source, wherein the inorganic carbon source comprises carbon dioxide. In a further embodiment, the process is for the production of biomass and comprises culturing the bacteria in continuous culture with hydrogen as energy source and an inorganic carbon source, wherein the inorganic carbon source comprises carbon dioxide. Various further embodiments of the process are described herein below.

In a further main aspect, the aspects of the disclosed embodiments relate to a process for the production of biomass and/or protein, said process comprising culturing a variant chemoautotrophic bacterial strain in continuous culture with hydrogen as energy source and an inorganic carbon source, wherein the inorganic carbon source comprises carbon dioxide and wherein said variant chemoautotrophic strain comprises a gene disruption of one or more genes encoding a PHA synthase, preferably the gene with most sequence identity to phaC1 (SEQ ID NO:62). In one embodiment, the process is for the production of biomass. In another embodiment, the process is for the production of protein. Various further embodiments of the process are described herein below.

In one embodiment, the variant chemoautotrophic strain used in the process is of the genus Xanthobacter, preferably a variant of strain VTT-E-193585.

In another embodiment, the variant chemoautotrophic strain used in the process is of the species Cupriavidus necator.

According to the genome sequence, the strain deposited under number VTT-E-193585 uses most likely Calvin-Benson-Bassham cycle for the carbon fixation where carbon dioxide molecule is connected to 5-carbon chain of ribulose 1,5-bisphosphate forming two molecules of glycerate 3-phosphate. This enables the strain to synthesise all the other organic molecules it requires for growth. Energy from hydrogen comes into the cell most likely through NAD⁺-reducing hydrogenases and/or NiFeSe-hydrogenases. In essence that is a redox reaction where hydrogen (H₂) is oxidized to H⁺ and NAD⁺ is reduced to NADH. In addition to ATP, NADH is one of the main energy carriers inside living organisms. Alternatively, some other energy equivalent is reduced by another hydrogenase enzyme using H₂. The Calvin-Benson-Bassham cycle requires energy in the form of ATP and NADH/NADPH in order to fix CO₂. The strain most likely generates ATP through oxidative phosphorylation, which consists of four protein complexes generating a proton gradient across a membrane. The proton gradient is generated using mainly energy from NADH. The proton gradient drives the ATP synthase complex generating ATP. According to the genome sequence, the strain has a bacterial F-type ATP synthase.

It is to be understood, when it is specified that the process comprises culturing the strain with an inorganic carbon source, that the inorganic carbon source is the main carbon source in the culture. Thus, there may be minor amounts of organic carbon sources present in the culture, but the main metabolism and growth of the culture is based on the utilisation of the inorganic carbon source, preferably carbon dioxide, as carbon source. Preferably the proportion of the carbon supplied to the culture that is organic is less than 5%, such as less than 1%, e.g. less than 0.1% of all carbon supplied to the culture during the process. Preferably, no organic carbon sources are supplied to the process.

Similarly, it is to be understood, when it is specified that the process comprises culturing the strain with hydrogen (H₂) as energy source, that hydrogen is the main energy source in the culture. Thus, there may be other minor energy sources present in the culture such as ammonia, which may be supplied as nitrogen source, or minor amounts of organic compounds, but the main metabolism and growth of the culture is based on the utilisation of hydrogen as energy source. In the overall process hydrogen is preferably produced by water electrolysis; i.e. by splitting water with electricity to hydrogen and oxygen gases. Thus, the hydrogen and oxygen gases are provided to the bioreactor from an electrolyser nearby. Alternatively, electrodes may be placed inside the bioreactor to produce hydrogen and oxygen in the bioreactor rather than in a separate electrolyser.

The inorganic carbon source comprising carbon dioxide may comprise other inorganic carbon sources, such as e.g. carbon monoxide. In one embodiment, only carbon sources in gaseous form are provided to the culture. In a preferred embodiment, carbon dioxide is the only inorganic carbon source, and indeed the only carbon source, provided to the culture. In one embodiment, only gases and minerals are provided to the culture and the level of carbon dioxide in the gas provided is between 10% and 50%, e.g. between 15% and 45%, such as between 20% and 40%, e.g. between 25% and 35%, such as between 26% and 30%.

In another embodiment, gases and minerals are provided to the culture and the level of hydrogen (H₂) in the gas provided is between 30% and 80%, e.g. between 35% and 75%, such as between 40% and 70%, e.g. between 45% and 65%, such as between 50% and 60%.

In another embodiment, gases and minerals are provided to the culture and the level of oxygen (O₂) in the gas provided is between 10% and 25%, e.g. between 15% and 20%, such as between 16% and 18%. In another embodiment, the level of oxygen provided is such that the level of dissolved oxygen in the culture is maintained at between 5% and 10%.

In a preferred embodiment, only gases and minerals are provided to the culture and the gas provided comprising H₂, CO₂ and O₂, wherein the percentage of H₂ is between 40% and 70%, the percentage of CO₂ is between 18% and 28% and the percentage of O₂ is between 12% and 22%.

Typically, the process of the aspects of the disclosed embodiments includes the addition of a nitrogen source. The nitrogen source may for example be provided in the form of ammonium hydroxide, an ammonium salt, such as ammonium sulphate or ammonium chloride, ammonia, urea or nitrate, e.g. potassium nitrate. In other embodiments, nitrogen gas (N₂) is provided as nitrogen source. In a preferred embodiment, the nitrogen source is ammonium hydroxide or an ammonium salt, such as ammonium sulphate.

In one embodiment, the nitrogen source provided is ammonium hydroxide at a concentration of between 100 mg/L and 10 g/L, such as between 250 mg/L and 4 g/L, e.g. between 0.5 g/L and 2 g/L, such as between 0.75 g/L and 1.5 g/L.

Typically, the process of the aspects of the disclosed embodiments includes the addition of minerals, such as minerals containing ammonium, phosphate, potassium, sodium, vanadium, iron, sulphate, magnesium, calcium, molybdenum, manganese, boron, zinc, cobalt, selenium, iodine, copper and/or nickel. Suitable mineral media are well-known art, and have e.g. been described in Thermophilic Bacteria, CRC Press, Boca Raton, F L, Jacob K. Kristjansson, ed., 1992, for example on page 87, Table 4.

In one embodiment, the minerals added include one or more of the following : ammonia, ammonium (e.g., ammonium chloride (NH₄Cl), ammonium sulphate ((NH₄)₂SO₄)), nitrate (e.g., potassium nitrate (KNO₃)), urea or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), disodium phosphate (Na₂HPO₄·2H₂O) dipotassium phosphate (K₂HPO₄) or monopotassium phosphate (KH₂PO₄); sulphate; yeast extract; chelated iron (chelated e.g. with EDTA or citric acid); potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulphate (MgSO₄·7H₂O) or magnesium chloride (MgCl₂), calcium chloride (CaCl₂), calcium sulphate (CaSO₄) or calcium carbonate (CaCO₃), manganese sulphate (MnSO₄·7H₂O) or manganese chloride (MnCl₂), ferric chloride (FeCl₂), ferrous sulphate (FeSO₄ 7H₂O) or ferrous chloride (FeCl₂ 4H₂O), sodium bicarbonate (NaHCO₃) or sodium carbonate (Na₂CO₃), zinc sulphate (ZnSO₄) or zinc chloride (ZnCl₂), ammonium molybdate (NH₄MoO₄) or sodium molybdate (Na₂MoO₄·2H₂O), cuprous sulphate (CuSO₄) or copper chloride (CuCl₂·2H₂O), cobalt chloride (CoCl₂·6H₂O) or cobalt sulphate (CoSO₄), aluminium chloride (AlCl₃·6H₂O), lithium chloride (LiCl), boric acid (H₃BO₃), nickel chloride NiCl₂·6H₂O) or nickel sulphate (NiSO₄), tin chloride (SnCl₂·H₂O), barium chloride (BaCl₂·2H₂O), copper selenate (CuSeO₄ 5H₂O), sodium selenate (NazSeO₄) or sodium selenite (Na₂SeO₃), sodium metavanadate (NaVO₃), chromium salts).

In a preferred embodiment, the process of the aspects of the disclosed embodiments includes the addition of one, more or all of: NH₄OH, KH₂PO₄, Na₂HPO₄·2H₂O, NaVO₃·H₂O, FeSO₄x7H₂O, MgSO₄·7H₂O, CaSO₄, Na₂MoO₄·2H₂O, MnSO₄·7H₂O, ZnSO₄·7H₂O, H₃BO₃, CoSO₄, CuSO₄, NiSO₄.

In one embodiment, the medium provided to the cells comprises less than 1 g/L of chloride salts, such as less than 0.25 g/L of chloride salts, e.g. less than 0.1 g/L of chloride salts, such as less than 0.025 g/L of chloride salts, e.g. less than 0.01 g/L of chloride. In one embodiment, no chloride salts are supplied to the culture.

In another embodiment, no vitamins are supplied during the process, i.e. the media provided to the culture does not contain vitamins.

In another embodiment, no amino acids are supplied during the process, i.e. the media provided to the culture does not contain amino acids.

In another embodiment, no organic compounds are supplied during the process, i.e. the media provided to the culture does not contain any organic compounds.

In certain embodiments, the pH of the bacterial culture is controlled at a certain level. In certain embodiments, pH is controlled within an optimal range for bacterial maintenance and/or growth and/or production of organic compounds. In one embodiment, the pH in the culture is maintained between 5.5 and 8.0, e.g. between 6.5 and 7.0, such as at 6.8.

In certain embodiments, the temperature of the bacterial culture is controlled. In certain embodiments, temperature is controlled within an optimal range for bacterial maintenance and/or growth and/or production of organic compounds. In one embodiment, the culture is grown at a temperature between 25° C. and 40° C., e.g. between 28° C. and 32° C., such as at 30° C.

Typically, the process of the aspects of the disclosed embodiments is carried out in a bioreactor. A bioreactor is utilized for the cultivation of cells, which may be maintained at particular phases in their growth curve. The use of bioreactors is advantageous in many ways for cultivating chemoautotrophic growth. Generally, the control of growth conditions, including control of dissolved carbon dioxide, oxygen, and other gases such as hydrogen, as well as other dissolved nutrients, trace elements, temperature and pH, is facilitated in a bioreactor. Nutrient media, as well as gases, can be added to the bioreactor as either a batch addition, or periodically, or in response to a detected depletion or programmed set point, or continuously while the period the culture is grown and/or maintained. In a continuous culture process, nutrient media, as well as gases, are added to the bioreactor continuously. Furthermore, bacteria-containing medium is being removed from the bioreactor continuously.

In a preferred embodiment, the volume of the bacterial culture is 100 ml or more, such as 1 L or more, e.g. 10 L or more, such as 100 L or more, e.g. 1,000 L or more, such as 10,000 L or more, e.g. 50,000 L or more, such as 100,000 L or more, e.g. 200,000 L or more.

In one embodiment, the productivity of the culture is more than 0.1 g cell dry weight per liter per hour, such as more than 0.2, e.g. more than 0.3, such as more than 0.4, e.g. more than 0.5, such as more than 0.6, e.g. more than 0.7, such as more than 0.8, e.g. more than 0.9, such as more than 1 g per liter per hour.

Bacteria can be inoculated directly from a cell bank, or via a seed culture at a smaller scale. Preferably, supply of fresh media to the culture and removal of used up media with bacteria is occurring at the same rate, such that the volume in the bioreactor remains the same.

In one embodiment, after an initial phase of reaching a suitable cell density, the bacteria grow at steady state or pseudo steady state, remaining continuously in their log phase, at an OD600 above 5, such as above 10, e.g. above 20, such as between 50 and 200, e.g. between 50 and 100.

In one embodiment of the process of the aspects of the disclosed embodiments, the bacterial strain has a growth rate of 0.001-0.12 h⁻¹, such as 0.01-0.12 h⁻¹, for example 0.04-0.12 h⁻¹.

In another embodiment of the process of the aspects of the disclosed embodiments, the liquid feed rate in the continuous phase is 50-80% of the growth rate.

Xanthobacter is a genus of Gram-negative bacteria from the Xanthobacteraceae family. As mentioned above, in one embodiment, the variant chemoautotrophic strain used in the process of the aspects of the disclosed embodiments (i.e. a variant that comprises a gene disruption of one or more genes encoding a PHA synthase) is of the genus Xanthobacter. Preferably, the strain is selected from the group consisting of: X. agilis, X. aminoxidans, X. autotrophicus, X. flavus, X. tagetidis, X. viscosus, Xanthobacter sp. 126, Xanthobacter sp. 91 and strain VTT-E-193585.

In one embodiment, the variant chemoautotrophic strain used in the process of the aspects of the disclosed embodiments is a strain which uses the Calvin Benson Bassham pathway to convert carbon dioxide into organic compounds, e.g. glucose, essential for living organisms.

In one embodiment, the variant chemoautotrophic strain used in the process of the aspects of the disclosed embodiments is a strain which uses NiFeSe-hydrogenases for converting hydrogen (H₂) into cellular energy equivalents.

In one embodiment, the variant chemoautotrophic strain used in the process of the aspects of the disclosed embodiments is a strain which uses NAD⁺-reducing hydrogenases for converting hydrogen (H₂) into cellular energy equivalents.

In one embodiment, the variant chemoautotrophic strain used in the process of the aspects of the disclosed embodiments is capable of nitrogen fixation.

In another embodiment, the variant chemoautotrophic bacterial strain used in the process of the aspects of the disclosed embodiments comprises the 16S ribosomal RNA set forth in SEQ ID NO:1 or a 16S ribosomal RNA having up to 20 nucleotide differences with SEQ ID NO:1, e.g. 1 to 10, such as 1 to 5, e.g. one, two or three nucleotide differences with SEQ ID NO:1.

In another embodiment, the variant chemoautotrophic bacterial strain used in the process of the aspects of the disclosed embodiments comprises a gene encoding a ribulose-1,5-bisphosphate carboxylase/oxygenase (rubisco) large chain having the sequence set forth in SEQ ID NO:3 or a sequence having more than more than 93% identity, e.g. more than 95% identity, such as more than 96% identity, e.g. more than 97% identity, such as more than 98% identity, e.g. more than 99% sequence identity to the sequence set forth in SEQ ID NO:3.

In another embodiment, the variant chemoautotrophic bacterial strain used in the process of the aspects of the disclosed embodiments comprises a gene encoding a ribulose-1,5-bisphosphate carboxylase/oxygenase (rubisco) small chain having the sequence set forth in SEQ ID NO:5 or a sequence having more than 83% sequence identity, e.g. more than 86%, identity such as more than 90% identity, e.g. more than 95% identity, such as more than 96% identity, e.g. more than 97% identity, such as more than 98% identity, e.g. more than 99% sequence identity to the sequence set forth in SEQ ID NO:5.

SEQ ID NO: 2:
Nucleotide sequence of Ribulose bisphosphate carboxylase large chain:
ATGGGTGCCGAAGCAACCGTCGGGCAGATCACGGACGCCAAGAAGAGATACGCCGCCGGCG
TGCTGAAGTACGCCCAGATGGGCTACTGGAACGGCGACTACGTTCCCAAGGACACCGACCTC
CTGGCGGTGTTCCGCATCACCCCCCAGGCGGGCGTGGACCCGGTGGAAGCCGCCGCGGCGG
TCGCCGGCGAAAGCTCCACCGCTACCTGGACCGTGGTGTGGACCGACCGGCTCACCGCCGC
CGACGTCTACCGCGCCAAGGCCTACAAGGTGGAGCCGGTGCCGGGCCAGGAAGGCCAGTAT
TTCTGCTACATCGCCTATGATCTCGATTTGTTCGAGGAAGGCTCCATCGCCAACCTCACGGCG
TCGATCATCGGCAACGTCTTCTCCTTCAAGCCGCTGAAGGCGGCGCGGCTGGAGGACATGCG
GCTTCCCGTCGCCTATGTGAAGACCTTCCGCGGCCCGCCCACCGGCATCGTGGTCGAGCGCG
AGCGCCTGGACAAGTTCGGCCGCCCCCTTCTGGGCGCCACCACCAAGCCGAAGCTTGGCCTC
TCGGGCAAGAATTACGGCCGCGTGGTCTATGAGGCCCTCAAGGGCGGCCTCGACTTCGTGAA
GGACGACGAGAACATCAACTCGCAGCCCTTCATGCACTGGCGCGATCGCTTCCTCTATTGCAT
GGAGGCCGTCAACAAGGCCCAGGCCGAGACCGGCGAGGTGAAGGGGCACTATCTCAACATC
ACCGCCGGGACCATGGAGGAGATGTACCGCCGCGCCGAGTTCGCCAAGGAACTGGGCTCCG
TGGTGGTGATGGTGGATCTCATCATCGGCTGGACCGCCATCCAGTCCATGTCCAACTGGTGC
CGCGAGAACGACATGATCCTGCACATGCACCGTGCGGGCCATGGCACCTACACGCGCCAGAA
GAGCCACGGCGTCTCCTTCCGCGTCATCGCCAAGTGGCTGCGGCTCGCCGGCGTCGACCACC
TGCACACCGGCACCGCCGTGGGCAAGCTGGAAGGCGACCCCATGACCGTGCAGGGCTTCTA
CAATGTCTGCCGCGAGACGACGACGCAGCAGGACCTCACCCGCGGCCTGTTCTTCGAGCAGG
ACTGGGGCGGCATCCGCAAGGTGATGCCGGTGGCCTCCGGCGGCATCCATGCGGGCCAGAT
GCACCAGCTCATCGACCTGTTCGGCGAGGACGTGGTGCTCCAGTTCGGCGGCGGCACCATCG
GCCACCCGGACGGCATCCAGGCCGGCGCCACCGCCAACCGCGTGGCGCTGGAAACCATGAT
CCTCGCCCGCAACGAGGGCCGCGACATCAGGAACGAGGGCCCGGAAATCCTGGTGGAAGCC
GCCAAATGGTGCCGTCCGCTGCGCGCGGCGCTCGATACCTGGGGCGAGGTGACCTTCAACTA
CGCCTCCACCGACACGTCCGATTACGTGCCCACCGCGTCCGTCGCCTGA
SEQ ID NO: 3:
Amino acid sequence of Ribulose bisphosphate carboxylase large chain
MGAEATVGQITDAKKRYAAGVLKYAQMGYWNGDYVPKDTDLLAVFRITPQAGVDPVEAAAAVA
GESSTATWTVVWTDRLTAADVYRAKAYKVEPVPGQEGQYFCYIAYDLDLFEEGSIANLTASIIGN
VFSFKPLKAARLEDMRLPVAYVKTFRGPPTGIVVERERLDKFGRPLLGATTKPKLGLSGKNYGRVV
YEALKGGLDFVKDDENINSQPFMHWRDRFLYCMEAVNKAQAETGEVKGHYLNITAGTMEEMYRR
AEFAKELGSVVVMVDLIIGWTAIQSMSNWCRENDMILHMHRAGHGTYTRQKSHGVSFRVIAKW
LRLAGVDHLHTGTAVGKLEGDPMTVQGFYNVCRETTTQQDLTRGLFFEQDWGGIRKVMPVASG
GIHAGQMHQLIDLFGEDVVLQFGGGTIGHPDGIQAGATANRVALETMILARNEGRDIRNEGPEIL
VEAAKWCRPLRAALDTWGEVTFNYASTDTSDYVPTASVA
SEQ ID NO: 4:
Nucleotide sequence of Ribulose bisphosphate carboxylase small chain:
ATGCGCATCACCCAAGGCTCCTTCTCCTTCCTGCCGGACCTCACCGACACGCAGATCAAGGCC
CAGGTGCAATATTGCCTGGACCAGGGCTGGGCGGTCTCGGTGGAGCACACCGACGATCCCCA
CCCGCGCAACACCTATTGGGAGATGTGGGGCCCGCCCATGTTCGATCTGCGCGACGCGGCC
GGCGTCTTCGGCGAGATCGAAGCCTGCCGGGCCGCCAATCCCGAGCATTATGTGCGGGTGAA
CGCCTTCGATTCCAGCCGCGGATGGGAGACGATCCGCCTGTCCTTCATCGTTCAGCGGCCCA
CCGTGGAAGAGGGCTTCCGCCTCGACCGCACCGAAGGCAAGGGCCGCAACCAGAGCTACGC
CATGCGCTACCGGGCGCAGTTCGCGCCGCGCTGA
SEQ ID NO: 5:
Amino acid sequence of Ribulose bisphosphate carboxylase small chain:
MRITQGSFSFLPDLTDTQIKAQVQYCLDQGWAVSVEHTDDPHPRNTYWEMWGPPMFDLRDAAG
VFGEIEACRAANPEHYVRVNAFDSSRGWETIRLSFIVQRPTVEEGFRLDRTEGKGRNQSYAMRYR
AQFAPR

In another embodiment, the variant chemoautotrophic bacterial strain used in the process of the aspects of the disclosed embodiments relate comprises a gene encoding a NAD⁺-reducing hydrogenase HoxS subunit alpha having the sequence set forth in SEQ ID NO:7 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:7.

In another embodiment, the variant chemoautotrophic bacterial strain used in the process of the aspects of the disclosed embodiments comprises a gene encoding a NAD⁺-reducing hydrogenase HoxS subunit beta having the sequence set forth in SEQ ID NO:9 or a sequence having more than 77% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:9.

In another embodiment, the variant chemoautotrophic bacterial strain used in the process of the aspects of the disclosed embodiments comprises a gene encoding a NAD⁺-reducing hydrogenase HoxS subunit gamma having the sequence set forth in SEQ ID NO:11 or a sequence having more than 70% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:11.

In another embodiment, the variant chemoautotrophic bacterial strain used in the process of the aspects of the disclosed embodiments comprises a gene encoding a NAD⁺-reducing hydrogenase HoxS subunit delta having the sequence set forth in SEQ ID NO:13 or a sequence having more than 79% sequence identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:13.

SEQ ID NO: 6:
Nucleotide sequence of NAD⁺-reducing hydrogenase HoxS subunit alpha:
ATGATGCCATCTGAGCCGCACGGCGCGGGCATGCCGCCCCCACGGGAAGCGGCCGCGGTTC
CCACCCCCCAGGAGGTGAGCGCGGTGGTGGCCGAGGTGGTCGCGGATGCCGTGGCATCGGT
GGGCGGCGCACGCACCCGGCTCATGGACATCGTCCAGCTGGCCCAGCAGCGTCTCGGCCAT
CTCTCCGAAGAGACCATGGCGGCCATTGCCGCGCGGCTCGCCATTCCGCCGGTGGAAGTGG
CGGACATGGTGTCCTTCTACGCCTTCCTGAACCGCGCGCCCAAGGGCCGCTACCACATCCGC
CTGTCGCGCAGCCCCATCTCGCTGATGAAGGGCGCCGAGGCGGTGGCTGCCGCCTTCTGCCA
GATCCTCGGCATCGCCATGGGCGAGACCTCGCAGGATGGCGACTTCACCCTGGAATGGACCA
ACGACATCGGCATGGCCGACCAGGAGCCGGCCGCCCTCGTCAACGGCACGGTGATGACGCA
GCTCGCGCCCGGCGATGCGGCCATCATCGTCGGCCGGCTGCGGGCCCATCACGCGCCCAAT
GCCCTGCCGCTGTTCCCTGGAGCCGGCGTGGCCGGCTCCGGCCTGCCCCATGCCCGGATCC
GCCCCAGCCTGGTGATGCCGGGACAGCTTCTGTTCCGCGAGGACCACACGACGCCGGGCGC
CGGCATCAAGGCGGCACTCGCCCTCACCCCGGACGAAGTGGTGCAGAAGGTCTCCGCCGCG
CGCCTGCGCGGGCGGGGTGGCGCCGGCTTTCCCACCGGTCTCAAATGGAAGCTCTGCCGCC
AGTCGCCCGCCACCACCCGCCATGTGATCTGCAATGCGGACGAGGGCGAGCCCGGCACCTTC
AAGGATCGCGTGCTGCTCACGCAGGCGCCGCACCTCATGTTCGACGGCATGACCATCGCCGG
CTACGCCTTGGGGGCGCGGGAGGGCGTGGTCTATCTGCGCGGCGAGTACGCCTATCTGTGG
GAGCCTCTGCATGCGGTCCTGCGCGAGCGCTATGGGCTCGGGCTCGCCGGCGCGAACATCC
TGGGACACGCGGGCTTCGACTTCGACATCCGCATCCAGCTGGGCGCCGGCGCCTATATCTGC
GGCGAGGAATCCGCGCTGGTGGAATCGCTGGAAGGCAAGCGCGGCTCGCCCCGCGACCGCC
CCCCCTTCCCCACCGTGCGCGGCCATCTCCAGCAGCCCACCGCCGTGGACAATGTGGAGACC
TTCGCCTGCGCCGCCCGCATCCTGGAGGATGGCGTGGAGGCGTTCGCGGGCATCGGCACGC
CCGAATCCGCCGGCACGAAGCTCCTCTCGGTGTCGGGCGATTGCCCGCGCCCCGGCGTGTAT
GAGGTGCCCTTCGGCCTCACGGTGAACGCGCTGCTCGACCTTGTCGGCGCGCCGGACGCCG
CCTTCGTGCAGATGGGTGGGCCGTCCGGCCAATGCGTGGCGCCGAAGGATTACGGCCGCCG
CATCGCCTTCGAGGACCTGCCCACCGGCGGCTCGGTGATGGTGTTCGGCCCGGGGCGCGAC
GTGCTCGCCATGGTGCGCGAGTTCGCGGATTTCTTCGCCGGCGAATCCTGCGGCTGGTGCAC
GCCCTGCCGGGTGGGCACCACCTTGCTCAAGGAAGAGCTGGACAAGCTCCTCGCCAACCGCG
CCACCCTCGCCGACATCCGCGCGCTGGAGACCCTGGCCACGACCGTCTCCCGCACCAGCCGC
TGCGGCCTCGGCCAGACGGCGCCCAACCCCATCCTTTCCACCATGCGCAACCTGCCGGAAGC
CTATGAGGCGAGGCTGAGGCCCGAAGACTTCCTGCCCTGGGCCTCGCTCGACGAGGCGCTG
AAGCCCGCCATCGTCATCCAGGGCCGCGCGCCCGTGCCGGAGGAAGAGGCATGA
SEQ ID NO: 7:
Amino acid sequence of NAD⁺-reducing hydrogenase HoxS subunit alpha:
MMPSEPHGAGMPPPREAAAVPTPQEVSAVVAEVVADAVASVGGARTRLMDIVQLAQQRLGHLSE
ETMAAIAARLAIPPVEVADMVSFYAFLNRAPKGRYHIRLSRSPISLMKGAEAVAAAFCQILGIAMG
ETSQDGDFTLEWTNDIGMADQEPAALVNGTVMTQLAPGDAAIIVGRLRAHHAPNALPLFPGAGV
AGSGLPHARIRPSLVMPGQLLFREDHTTPGAGIKAALALTPDEVVQKVSAARLRGRGGAGFPTGL
KWKLCRQSPATTRHVICNADEGEPGTFKDRVLLTQAPHLMFDGMTIAGYALGAREGVVYLRGEY
AYLWEPLHAVLRERYGLGLAGANILGHAGFDFDIRIQLGAGAYICGEESALVESLEGKRGSPRDRP
PFPTVRGHLQQPTAVDNVETFACAARILEDGVEAFAGIGTPESAGTKLLSVSGDCPRPGVYEVPFG
LTVNALLDLVGAPDAAFVQMGGPSGQCVAPKDYGRRIAFEDLPTGGSVMVFGPGRDVLAMVREF
ADFFAGESCGWCTPCRVGTTLLKEELDKLLANRATLADIRALETLATTVSRTSRCGLGQTAPNPIL
STMRNLPEAYEARLRPEDFLPWASLDEALKPAIVIQGRAPVPEEEA
SEQ ID NO: 8:
Nucleotide sequence of NAD⁺-reducing hydrogenase HoxS subunit beta:
ATGAGCCGGGGATCCCCCGATGCCGGGAAAGACCGCACCATGAGCGCCACCGACGGCACCA
CCGCCCCCCGCAAGATCGTCATCGATCCGGTGACCCGCGTGGAGGGCCACGGCAAGGTCAC
CATCCGCCTGGATGAAGCCGGCGCGGTGGAGGATGCGCGTTTCCACATCGTGGAGTTCCGC
GGCTTCGAGCGGTTCATCCAGGGCCGGATGTACTGGGAAGTGCCCCTTATCATCCAGCGGCT
GTGCGGCATCTGCCCGGTGAGCCACCATCTGGCGGCGGCGAAAGCCATGGACCAGGTGGCG
GGCGTGGACCGCGTACCGCCCACCGCCGAGAAACTGCGCCGGCTGATGCATTATGGGCAGG
TGCTGCAATCCAACGCTTTGCACATCTTCCACCTCGCCTCGCCCGACCTCCTGTTCGGCTTCG
ACGCGCCGGCCGAGCAGCGCAACATCATCGCCGTGCTCCAGCGTTATCCGGAGATCGGCAAA
TGGGCGATCTTCATCAGGAAGTTCGGCCAGGAGGTCATCAAGGCCACCGGCGGGCGCAAGA
TCCATCCCACCAGCGCCATTCCCGGCGGGGTCAACCAGAACCTCGCCGTGGAGGACCGCGAC
GCCCTGCGCGCCAAGGTGGGCGAGATCATCAGCTGGTGCATGGCGGCGCTGGACCATCACA
AGGCCTATGTGGCGGAAAACCGGGCGCTGCATGACAGCTTCGCCGCCTTCCCCTCCGCCTTC
ATGAGCCTCGTGGGGCCGGATGGCGGCATGGACCTTTATGACGGCACCCTGCGGGTGATCG
ATGCCGAGGGCGCCCCCCTCATCGAAGGCGCGCCGCCCGCCTCCTACCGCGACCACCTCATC
GAGGAGGTGCGGCCCTGGAGCTATCTGAAATTCCCCCATCTGCGCGCCTTCGGCCGCGACGA
TGGCTGGTATCGGGTCGGCCCCCTCGCCCAGGTCAATTGCGCCGCGTCCATCGACACGCCCC
GCGCCGAGGCGGCCCGGCGGGACTTCATGGCCGAGGGGGGGGCAAGCCGGTGCATGCCA
CCCTCGCTTATCACTGGGCGCGGCTCATCGTGCTGGTCCATTGCGCGGAGAAGATCGAACAG
CTGCTGTTCGACGACGACCTGCAAGGCTGCGATCTGCGTGCGGAGGGCACCCGGCGCGGGG
AAGGCGTCGCCTGGATCGAGGCGCCGCGCGGCACCCTCATCCACCATTACGAGGTGGACGA
GAACGACCAGGTGCGCCGCGCCAACCTCATCGTCTCCACCACCCACAATAACGAGGCCATGA
ACCGCGCCGTGCGGCAGGTGGCGAAGACGGACCTTTCCGGTCGCGAGATCACCGAAGGGCT
GCTGAACCATATCGAGGTGGCCATCCGCGCCTTCGACCCCTGCCTGTCCTGCGCCACCCATG
CGCTGGGCCAGATGCCGCTGATCGTGACGCTTGAAGATGCCTCCGGCGCAGAGATCGCCCG
CGGAGTGAAGGAATGA
SEQ ID NO: 9:
Amino acid sequence of NAD⁺-reducing hydrogenase HoxS subunit beta:
MSRGSPDAGKDRTMSATDGTTAPRKIVIDPVTRVEGHGKVTIRLDEAGAVEDARFHIVEFRGFER
FIQGRMYWEVPLIIQRLCGICPVSHHLAAAKAMDQVAGVDRVPPTAEKLRRLMHYGQVLQSNAL
HIFHLASPDLLFGFDAPAEQRNIIAVLQRYPEIGKWAIFIRKFGQEVIKATGGRKIHPTSAIPGGVN
QNLAVEDRDALRAKVGEIISWCMAALDHHKAYVAENRALHDSFAAFPSAFMSLVGPDGGMDLY
DGTLRVIDAEGAPLIEGAPPASYRDHLIEEVRPWSYLKFPHLRAFGRDDGWYRVGPLAQVNCAAS
IDTPRAEAARRDFMAEGGGKPVHATLAYHWARLIVLVHCAEKIEQLLFDDDLQGCDLRAEGTRRG
EGVAWIEAPRGTLIHHYEVDENDQVRRANLIVSTTHNNEAMNRAVRQVAKTDLSGREITEGLLN
HIEVAIRAFDPCLSCATHALGQMPLIVTLEDASGAEIARGVKE
SEQ ID NO: 10:
Nucleotide sequence of NAD⁺-reducing hydrogenase HoxS subunit gamma:
ATGAGCGAGACCCCCTTCACCTTTACCGTGGACGGCATCGCGGTCCCGGCCACCCCCGGCCA
GAGCGTCATCGAGGCGTGCGATGCGGCGGGCATCTATATCCCGCGCCTGTGCCACCACCCG
GACCTGCCGCCGGCGGGCCATTGCCGGGTGTGCACCTGCATCATCGACGGGCGGCCGGCCA
GCGCCTGCACCATGCCCGCCGCCAGGGGCATGGTGGTGGAGAACGAGACGCCCGCTTTGCT
GGCGGAGCGGCGCACGCTGATCGAGATGCTGTTCGCGGAAGGCAACCATTTCTGCCAGTTCT
GCGAGGCGAGCGGCGATTGCGAATTGCAGGCGCTGGGCTACCTGTTCGGCATGGTGGCCCC
GCCCTTCCCCCATCTGTGGCCGAAGCGGCCGGTGGATGCCAGCCATCCGGATATCTATATCG
ACCACAATCGCTGCATCCTGTGCTCGCGCTGCGTGCGCGCCTCGCGCACCCTGGACGGCAAG
TCCGTGTTCGGCTTCGAGGGGCGCGGCATCGAGATGCATCTGGCGGTGACCGGCGGGCACC
TGGACGACAGCGCCATCGCCGCCGCCGACAGGGCGGTTGAGATGTGCCCGGTGGGCTGCAT
CGTCCTCAAGCGCACCGGCTACCGCACGCCCTATGGCCGGCGGCGCTACGACGCCGCGCCC
ATCGGCTCCGACATCACCGCCCGGCGCGGCGGCGCGAAGGACTGA
SEQ ID NO: 11:
Amino acid sequence of NAD⁺-reducing hydrogenase HoxS subunit gamma:
MSETPFTFTVDGIAVPATPGQSVIEACDAAGIYIPRLCHHPDLPPAGHCRVCTCIIDGRPASACTM
PAARGMVVENETPALLAERRTLIEMLFAEGNHFCQFCEASGDCELQALGYLFGMVAPPFPHLWPK
RPVDASHPDIYIDHNRCILCSRCVRASRTLDGKSVFGFEGRGIEMHLAVTGGHLDDSAIAAADRA
VEMCPVGCIVLKRTGYRTPYGRRRYDAAPIGSDITARRGGAKD
SEQ ID NO: 12:
Nucleotide sequence of NAD⁺-reducing hydrogenase HoxS subunit delta:
ATGGCCAAGCCCAAACTCGCCACCTGCGCGCTGGCCGGCTGCTTCGGCTGCCACATGTCCTT
CCTGGACATGGACGAGCGCATCGTCGAGCTCATCGACCTGGTGGACCTCGACGTCTCGCCCC
TCGACGACAAGAAAAACTTCACCGGCATGGTGGAAATCGGCCTGGTGGAAGGCGGCTGCGC
CGACGAGCGCCATGTGAAGGTGCTGCGCGAGTTCCGCGAGAAATCCCGCATCCTGGTGGCG
GTGGGCGCCTGCGCCATCACCGGCGGCATCCCGGCATTGCGCAACCTCGCCGGCCTCGACG
AATGCCTGAGGGAAGCCTACCTCACCGGCCCCACGGTGGAAGGCGGCGGGCTCATTCCCAAC
GACCCGGAGCTGCCGCTGCTGCTGGACAAGGTCTATCCGGTGCAGGACTTCGTGAAGATCGA
CCATTTCCTGCCCGGCTGCCCGCCCTCGGCCGACGCCATCTGGGCGGCTCTGAAGGCGCTGC
TGACCGGCACCGAGCCGCATCTGCCCTACCCGCTTTTCAAGTACGAATGA
SEQ ID NO: 13:
Amino acid sequence of NAD⁺-reducing hydrogenase HoxS subunit delta:
MAKPKLATCALAGCFGCHMSFLDMDERIVELIDLVDLDVSPLDDKKNFTGMVEIGLVEGGCADER
HVKVLREFREKSRILVAVGACAITGGIPALRNLAGLDECLREAYLTGPTVEGGGLIPNDPELPLLLD
KVYPVQDFVKIDHFLPGCPPSADAIWAALKALLTGTEPHLPYPLFKYE

In another embodiment, the variant chemoautotrophic bacterial strain used in the process of the aspects of the disclosed embodiments comprises a gene encoding a NiFeSe hydrogenase large subunit having the sequence set forth in SEQ ID NO:15 or a sequence having more than 84% sequence identity, e.g. more than 90% identity, such as more than 95% identity, e.g. more than 96% identity, such as more than 97% identity, e.g. more than 98%, such as more than 99% sequence identity to the sequence set forth in SEQ ID NO:15.

In another embodiment, the variant chemoautotrophic bacterial strain used in the process of the aspects of the disclosed embodiments comprises a gene encoding a NiFeSe hydrogenase small subunit having the sequence set forth in SEQ ID NO:17 or a sequence having more than 90% identity, such as more than 95% identity, e.g. more than 96% identity, such as more than 97% identity, e.g. more than 98%, such as more than 99% sequence identity to the sequence set forth in SEQ ID NO:17.

SEQ ID NO: 14:
Nucleotide sequence of Periplasmic [NiFeSe] hydrogenase large subunit:
TCCAGACCCGGGCAACATTGCTCCATGTGCTGGGCACCCTGGCCGGCCGCTGGCCCCATACC
CTCGCGCTCCAGCCCGGCGGGGTGACCCGAAGCGCCGACCAGCACGACCGCATGCGCCTGC
TCGCGACGCTGAAGGCGGTGCGGGCGGCGCTGGAAGAGACCTTGTTCGGCGCGCCTTTGGA
AGAGGTGGCGGCCCTGGACGGCGCCGCCGCCGTGGAGGCCTGGCGCGCCAACGGCCCGGA
AGGGGATTTCCGCCTGTTCCTGGAGATCGCCGCCGACCTGGAGCTGGACCGGCTCGGCCGC
GCGCACGACCGCTTTCTCTCCTTCGGCGCCTACGCCCAGGACGAGGGGCGCCTTTATGGCGC
CGGCACCTTCGAGGCCGGGACGGCGGGAGGGCTCGATCCCAACGCCATCACCGAGGACCAC
GCCTTCGCCCGCATGGAGGACCGCGCGGCGCCCCATGCGCCCTTTGACGGCTCCACCTTCCC
CGATGCCGACGACACCGAGGGCTACACCTGGTGCAAGGCGCCGCGCCTTGCCGGCCTGCCC
TTCGAGACCGGCGCCTTCGCCCGGCAGGTGGTGGCGGGCCATCCGCTCGCCCGGGACCTCG
TGACGCGGGAAGGCGGCACTGTGCGCAGCCGCGTGGTCGGCCGGCTGCTGGAAACCGCGC
GCACCCTGATCGCCATGGAGGGCTGGGTGAAGGAACTGCGGCCCGAAGGGCCCTGGTGCGC
CCAGGGCCACCTGCCCCAGGAAGGCCGCGCCTTCGGCCTCACCGAGGCGGCGCGCGGGGC
GCTCGGCCACTGGATGGTGGTGGAGAAGGGCCGCATTGCCCGCTACCAGATCATCGCCCCCA
CCACCTGGAACTTCTCCCCCCGCGACGGCGCGGGCCTGCCCGGCCCGCTGGAGACGGCCCT
GGTGGGCGCGCCCGTGCGGCAGGGAGAGACGACGCCCGTGAGCGTGCAGCACATCGTGCG
CTCCTTCGACCCGTGCATGGTCTGCACTGTGCATTGA
SEQ ID NO: 15:
Amino acid sequence of Periplasmic [NiFeSe] hydrogenase large subunit:
MSAETRRLVVGPFNRVEGDLEVRLDVQDGRVQQAFVSSPLFRGFERILEGRDPRDALVIAPRICGI
CSVSQSHAAALALAGLQGIAPTHDGRIATNLIVAAENVADHLTHFHVFFMPDFARAVYEDRPWFA
QAARRFKANQGVSVRRALQTRATLLHVLGTLAGRWPHTLALQPGGVTRSADQHDRMRLLATLKA
VRAALEETLFGAPLEEVAALDGAAAVEAWRANGPEGDFRLFLEIAADLELDRLGRAHDRFLSFGAY
AQDEGRLYGAGTFEAGTAGGLDPNAITEDHAFARMEDRAAPHAPFDGSTFPDADDTEGYTWCK
APRLAGLPFETGAFARQVVAGHPLARDLVTREGGTVRSRVVGRLLETARTLIAMEGWVKELRPEG
PWCAQGHLPQEGRAFGLTEAARGALGHWMVVEKGRIARYQIIAPTTWNFSPRDGAGLPGPLETA
LVGAPVRQGETTPVSVQHIVRSFDPCMVCTVH
SEQ ID NO: 16:
Nucleotide sequence of Periplasmic [NiFeSe] hydrogenase small subunit
ACGGGGGAGGAAGCCCGCGCCATCTTCGACGCCATCCTTGCCGGCGTTATCGTCCTCGACGC
CCTGTGCGTGGAAGGCGCGCTGCTGCGCGGGCCGAACGGCACCGGGCGCTTCCATGTGCTG
GCGGGCACGGACACCCCCACCATCGACTGGGCGCGGCAGCTCGCCGGCATGGCGCGCCACG
TGGTGGCGGTGGGCACCTGCGCCGCCTATGGGGGCGTGACGGCGGCGGGCATCAACCCCAC
CGATGCCTGCGGCCTCCAGTTCGACGGACGCCGGAAGGGTGGGGCGCTGGGGGCGGACTTC
CGCTCCCGCTCGGGGCTTCCGGTCATCAATGTGGCCGGCTGCCCCACCCATCCCAACTGGGT
GACGGAAACCCTGATGCTGCTCGCCTGCGGCCTGCTGGGCGAGGCCGACCTCGACGTCTATG
GCCGCCCGCGCTTCTATGCGGACCTGCTGGTGCATCACGGCTGCCCGCGCAACGAATACTAT
GAATACAAGGCGAGCGCCGAGAAGATGAGCGACCTCGGCTGCATGATGGAGCATCTGGGCT
GCCTCGGCACCCAGGCCCACGCCGACTGCAACACGCGCCTTTGGAATGGCGAGGGCTCGTG
CACCCGCGGCGGCTATGCCTGCATCAACTGCACGGCGCCGGAATTCGAGGAGCCGGGCCAC
GCCTTCCTGGAGACGCCCAAGATCGGCGGCATCCCCATCGGCCTGCCCACCGACATGCCCAA
GGCCTGGTTCATCGCCTTGTCCTCCCTCGCCAAGGCGGCGACGCCGGAGCGGCTGCGCAAG
AACGCGGTGTCCGACCATGTGGTCACGCCGCCCGCCGTCAAGGACATCAAGCGGCGATGA
SEQ ID NO: 17:
Amino acid sequence of Periplasmic [NiFeSe] hydrogenase small subunit
MSTPFSVLWLQSGGCGGCTMSLLCAEAPDLATTLDAAGIGFLWHPALSEETGEEARAIFDAILAG
VIVLDALCVEGALLRGPNGTGRFHVLAGTDTPTIDWARQLAGMARHVVAVGTCAAYGGVTAAGI
NPTDACGLQFDGRRKGGALGADFRSRSGLPVINVAGCPTHPNWVTETLMLLACGLLGEADLDVY
GRPRFYADLLVHHGCPRNEYYEYKASAEKMSDLGCMMEHLGCLGTQAHADCNTRLWNGEGSCT
RGGYACINCTAPEFEEPGHAFLETPKIGGIPIGLPTDMPKAWFIALSSLAKAATPERLRKNAVSDHV
VTPPAVKDIKRR

In another embodiment, the variant chemoautotrophic bacterial strain used in the process of the aspects of the disclosed embodiments comprises a gene encoding an ATP synthase gamma chain atpG_1 having the sequence set forth in SEQ ID NO:19 or a sequence having more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:19.

In another embodiment, the variant chemoautotrophic bacterial strain used in the process of the aspects of the disclosed embodiments comprises a gene encoding an ATP synthase subunit alpha atpA_1 having the sequence set forth in SEQ ID NO:21 or a sequence having more than 78% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:21.

In another embodiment, the variant chemoautotrophic bacterial strain used in the process of the aspects of the disclosed embodiments comprises a gene encoding an ATP synthase subunit b atpF_1 having the sequence set forth in SEQ ID NO:23 or a sequence having more than 62% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:23.

In another embodiment, the variant chemoautotrophic bacterial strain used in the process of the aspects of the disclosed embodiments comprises a gene encoding an ATP synthase subunit c, sodium ion specific atpE_1 having the sequence set forth in SEQ ID NO:25 or a sequence having more than 90% identity, such as more than 95% identity, e.g. more than 96% identity, such as more than 97% identity, e.g. more than 98%, such as more than 99% sequence identity to the sequence set forth in SEQ ID NO:25.

In another embodiment, the variant chemoautotrophic bacterial strain used in the process of the aspects of the disclosed embodiments comprises a gene encoding an ATP synthase subunit a atpB_1 having the sequence set forth in SEQ ID NO:27 or a sequence having more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:27.

In another embodiment, the variant chemoautotrophic bacterial strain used in the process of the aspects of the disclosed embodiments comprises a gene encoding an ATP synthase epsilon chain atpC_1 having the sequence set forth in SEQ ID NO:29 or a sequence having more than 71% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:29.

In another embodiment, the variant chemoautotrophic bacterial strain used in the process of the aspects of the disclosed embodiments comprises a gene encoding an ATP synthase subunit beta atpD_1 having the sequence set forth in SEQ ID NO:31 or a sequence having more than 84% identity, e.g. more than 90% identity, such as more than 95% identity, e.g. more than 96% identity, such as more than 97% identity, e.g. more than 98%, such as more than 99% sequence identity to the sequence set forth in SEQ ID NO:31.

In another embodiment, the variant chemoautotrophic bacterial strain used in the process of the aspects of the disclosed embodiments comprises a gene encoding an ATP synthase subunit beta atpD_2 having the sequence set forth in SEQ ID NO:33 or a sequence having more than 97% identity, e.g. more than 98%, such as more than 99% sequence identity to the sequence set forth in SEQ ID NO:33.

In another embodiment, the variant chemoautotrophic bacterial strain used in the process of the aspects of the disclosed embodiments comprises a gene encoding an ATP synthase gamma chain atpG_2 having the sequence set forth in SEQ ID NO:35 or a sequence having more than 86% identity, e.g. more than 90% identity, such as more than 95% identity, e.g. more than 96% identity, such as more than 97% identity, e.g. more than 98%, such as more than 99% sequence identity to the sequence set forth in SEQ ID NO:35.

In another embodiment, the variant chemoautotrophic bacterial strain used in the process of the aspects of the disclosed embodiments comprises a gene encoding an ATP synthase subunit alpha atpA_2 having the sequence set forth in SEQ ID NO:37 or a sequence having more than 98%, such as more than 99% sequence identity to the sequence set forth in SEQ ID NO:37.

In another embodiment, the variant chemoautotrophic bacterial strain used in the process of the aspects of the disclosed embodiments comprises a gene encoding an ATP synthase subunit delta atpH having the sequence set forth in SEQ ID NO:39 or a sequence having more than 85% identity, e.g. more than 90% identity, such as more than 95% identity, e.g. more than 96% identity, such as more than 97% identity, e.g. more than 98%, such as more than 99% sequence identity to the sequence set forth in SEQ ID NO:39.

In another embodiment, the variant chemoautotrophic bacterial strain used in the process of the aspects of the disclosed embodiments comprises a gene encoding an ATP synthase subunit b atpF_2 having the sequence set forth in SEQ ID NO:41 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:41.

In another embodiment, the variant chemoautotrophic bacterial strain used in the process of the aspects of the disclosed embodiments comprises a gene encoding an ATP synthase subunit b′ atpG_3 having the sequence set forth in SEQ ID NO:43 or a sequence having more than 81% identity, e.g. more than 90% identity, such as more than 95% identity, e.g. more than 96% identity, such as more than 97% identity, e.g. more than 98%, such as more than 99% sequence identity to the sequence set forth in SEQ ID NO:43.

In another embodiment, the variant chemoautotrophic bacterial strain used in the process of the aspects of the disclosed embodiments comprises a gene encoding ATP synthase subunit c atpE_2 having the sequence set forth in SEQ ID NO:45 or a sequence having more than 98%, such as more than 99% sequence identity to the sequence set forth in SEQ ID NO:45.

In another embodiment, the variant chemoautotrophic bacterial strain used in the process of the aspects of the disclosed embodiments comprises a gene encoding an ATP synthase subunit a atpB_2 having the sequence set forth in SEQ ID NO:47 or a sequence having more than 92% identity, such as more than 95% identity, e.g. more than 96% identity, such as more than 97% identity, e.g. more than 98%, such as more than 99% sequence identity to the sequence set forth in SEQ ID NO:47.

In another embodiment, the variant chemoautotrophic bacterial strain used in the process of the aspects of the disclosed embodiments comprises a gene encoding an ATP synthase protein I atpI having the sequence set forth in SEQ ID NO:49 or a sequence having more than 60% identity, e.g. more than 70% identity, such as more than 80% identity, e.g. more than 90% identity, such as more than 95% identity, e.g. more than 96% identity, such as more than 97% identity, e.g. more than 98%, such as more than 99% sequence identity to the sequence set forth in SEQ ID NO:49.

SEQ ID NO: 18:
Nucleotide sequence of ATP synthase gamma chain atpG_1
GTGACCGAGCGCCTGTCCGACGTCAACGCCCGCATCGCCTCGGTGCGGCAGCTCTCATCGGT
CATCACGGCCATGCGGGGCATTGCGGCGGCGCGGGCGCGGGAGGCGCGGGGTCGGCTCGA
CGGCATCCGCGCCTATGCGCAGACCATCGCCGAGGCCATCGGCCATGTGCTCGCCGTGCTGC
CCGAGGAGGCCCGCGCCCGGTCCTCCGGGCACCGGCATCGGGGCCATGCGGTCATCGCCCT
GTGCGCGGAGCAGGGCTTTGCCGGCGTCTTCAACGAGCGGGTGCTGGACGAGGCCGCCCGG
CTGCTGACCGGCGGGGCGGGGCCGGCCGAGCTGCTGCTGGTGGGCGACCGGGGCCTGATG
GTGGCCCGCGAGCGGGGGCTCGATGTCTCCTGGTCGGTGCCCATGGTGGCCCATGCGGGCC
AGGCCTCGGCGCTGGCGGACCGCATCAGCGAGGAGCTCTACCGGCGGATCGATGCGGGACG
GGTGACGCGGGTGTCGGTGGTGCACGCCGAGCCCGCCGCGTCCGCCGCCATCGAGACGGTG
GTGAAAGTGCTGGTGCCGTTCGACTTCGCCCGCTTCCCCCTGGCGCGGGTGGCATCCGCCCC
GCTCATGACCATGCCGCCGCCGCGGCTGCTGGCCCAGCTGTCGGAGGAATATGTGTTCGCCG
AGCTGTGCGAGGCGCTCACCTTGTCCTTCGCGGCGGAGAACGAGGCCCGCATGCGGGCCAT
GATCGCCGCCCGCGCCAATGTGGCCGATACCCTGGAGGGCCTCGTCGGCCGCGCCCGGCAG
ATGCGCCAGGAGGAGATCACCAACGAGATCATCGAGCTGGAAGGCGGCGCCGGCAGCGCCC
GGCATGCGGATTGA
SEQ ID NO: 19:
Amino acid sequence of ATP synthase gamma chain atpG_1
MTERLSDVNARIASVRQLSSVITAMRGIAAARAREARGRLDGIRAYAQTIAEAIGHVLAVLPEEAR
ARSSGHRHRGHAVIALCAEQGFAGVFNERVLDEAARLLTGGAGPAELLLVGDRGLMVARERGLD
VSWSVPMVAHAGQASALADRISEELYRRIDAGRVTRVSVVHAEPAASAAIETVVKVLVPFDFARF
PLARVASAPLMTMPPPRLLAQLSEEYVFAELCEALTLSFAAENEARMRAMIAARANVADTLEGLVG
RARQMRQEEITNEIIELEGGAGSARHAD
SEQ ID NO: 20:
Nucleotide sequence of ATP synthase subunit alpha atpA_1
ATGAGCACGGGCGCGCAAGCGAGCGAGGATTGGCTCACCCGGAGCCGGGCGGCCCTGGCC
GGGACGCGCCTTTCCCAGCAATCCCAATCGGTGGGCCGGGTGGAGGAGATGGCCGACGGCA
TCGCCCGCGTCTCCGGCCTGCCGGATGTGCGGCTCGACGAGCTTCTCACCTTCGAGGGCGGC
CAGACCGGCTATGCCCTCACCCTCGATCGCACCGAGATCGCCGTGGTGCTGCTGGATGACGC
CTCCGGCGTGGAGGCGGGCGCCCGGGTGTTCGGCACCGGCGAGGTGGTGAAGGTGCCGGT
GGGGCCGGGGCTGCTGGGCCGCATCGTCGACCCCCTCGGCCGGCCCATGGACCGCTCCGAG
CCGGTGGTGGCGCAGGCGCACCATCCCATCGAGCGGCCGGCGCCGGCCATCATCGCCCGCG
ACCTGGTCTCGCAGCCGGTTCAGACCGGCACGCTGGTGGTGGATGCGCTGTTCTCCCTCGGC
CGGGGCCAGCGCGAGCTCATCATCGGCGACCGGGCTACCGGCAAGACCGCCATCGCGGTGG
ACACCATCATCAGCCAGAAGCATTCGGACATCGTGTGCATCTACGTGGCGGTGGGCCAGCGC
GCCGCCGCCGTGGAGCGGGTGGTGGAGGCGGTGCGCGCCCACGGGGCGATCGAGCGCTGC
ATCTTCGTGGTCGCCTCGGCCGCCGCCTCGCCAGGGCTGCAATGGATCGCGCCGTTCGCCGG
CATGACCATGGCGGAATATTTCCGCGACAACGGCCAGCATGCGCTCATCATCATCGATGATCT
CACCAAGCATGCGGCCACCCATCGCGAGCTGGCGCTGCTCACCCACGAGCCGCCGGGCCGC
GAGGCCTATCCCGGCGACATCTTCTATGTGCACGCCCGCCTTCTGGAGCGGGCCGCCAAGCT
CTCCGCCGAGCTGGGCGGTGGCTCGCTCACGGCCCTGCCCATCGCGGAGACGGACGCGGGA
AACCTCTCCGCCTATATCCCCACCAACCTCATCTCCATCACCGATGGGCAGATCGTGCTGGAT
TCGCGGCTGTTCGCGGCCAACCAGCGCCCGGCGGTGGATGTGGGCCTCTCCGTGAGCCGGG
TGGGCGGCAAGGCGCAGCATCCCGCGCTTCGGGCCGTGTCCGGGCGCATCCGGCTCGATTA
TTCCCAGTTCCTGGAGCTGGAAATGTTCACCCGCTTCGGCGGCATCACCGATACCCGCGTGAA
GGCGCAGATCACCCGGGGCGAGCGCATCCGCGCGCTGCTCACCCAGCCGCGCTTTTCCACCC
TGCGCCTTCAGGACGAGGTGGCGCTGCTGGCCGCGCTGGCGGAGGGGGTGTTCGACACTTT
GGCCCCGGGGCTGATGGGCGCCGTGCGTGCCCGCATTCCGGCCCAGCTGGATGCGCAGGTG
AAGGACGTGGCCTCGGCCCTCGCCGAGGGCAAGGTGCTGGAGGAGGGCTTGCACGCCCGTC
TCGTGGCGGCCGTGCGGGCCGTCGCGGCGGACGTGGCCGCGACCGCGAAGGCCGGGCCGT
GA
SEQ ID NO: 21:
Amino acid sequence of ATP synthase subunit alpha atpA_1
MSTGAQASEDWLTRSRAALAGTRLSQQSQSVGRVEEMADGIARVSGLPDVRLDELLTFEGGQT
GYALTLDRTEIAVVLLDDASGVEAGARVFGTGEVVKVPVGPGLLGRIVDPLGRPMDRSEPVVAQA
HHPIERPAPAIIARDLVSQPVQTGTLVVDALFSLGRGQRELIIGDRATGKTAIAVDTIISQKHSDIV
CIYVAVGQRAAAVERVVEAVRAHGAIERCIFVVASAAASPGLQWIAPFAGMTMAEYFRDNGQHA
LIIIDDLTKHAATHRELALLTHEPPGREAYPGDIFYVHARLLERAAKLSAELGGGSLTALPIAETDAG
NLSAYIPTNLISITDGQIVLDSRLFAANQRPAVDVGLSVSRVGGKAQHPALRAVSGRIRLDYSQFL
ELEMFTRFGGITDTRVKAQITRGERIRALLTQPRFSTLRLQDEVALLAALAEGVFDTLAPGLMGAV
RARIPAQLDAQVKDVASALAEGKVLEEGLHARLVAAVRAVAADVAATAKAGP
SEQ ID NO: 22:
Nucleotide sequence of ATP synthase subunit b atpF_1
ATGCAGATCGACTGGTGGACGCTGGGCCTGCAGACGGTCAACGTCCTCGTTCTCATCTGGCT
CCTGAGCCGCTTCCTGTTCAAGCCGGTGGCGCAGGTCATCGCGCAGCGCCGTGCCGAGATCG
AGAAGCTGGTGGAGGATGCGCGCGCCGCCAAGGCCGCCGCCGAGGCCGAGCGGGACACGG
CGAAGGCGGAGGAGGCGCGCCTTGCCGCCGAGCGCGGCGCCCGCATGGCGGCGGTCGCCA
AGGAGGCGGAGGCGCAGAAGGCGGCATTGCTGGCCGCCGCCAAGACCGAGGCCGAGGCCC
TGCACGCGGCCGCGGAAGCGGCCATCGTCCGGGCGCGGGCGAGCGAGGAGGAAGCCGCCG
CCGACCGCGCCAGCCGCCTTGCCGTGGACATCGCCGCCAAGCTGCTGGACCGGCTGCCCGA
CGACGCCCGGGTCGCGGGCTTCATCGATGGCCTCGCCGAGGGGCTTGAAGCCCTGCCCGAG
GCGAGCCGGGCGGTGATCGGCGTCGACGGCGCGCCAGTGCGCGTGACGGCCGCGCGCGCC
CTTATGCCGGCGGAGGAGGAGGCCTGCCGCACGCGGCTCTCCCAGGCGCTGGGCCGTCCGG
TGACGCTGGCCGTGACCATCGACCCCGCCCTCATCGCCGGCCTGGAGATGGAGACGCCCCAC
GCGGTGGTGCGCAATTCCTTCAAGGCCGATCTCGACCGCGTCACCGCGGCGCTCACCCATCA
TGGGACCTGA
SEQ ID NO: 23:
Amino acid sequence of ATP synthase subunit b atpF_1
MQIDWWTLGLQTVNVLVLIWLLSRFLFKPVAQVIAQRRAEIEKLVEDARAAKAAAEAERDTAKAE
EARLAAERGARMAAVAKEAEAQKAALLAAAKTEAEALHAAAEAAIVRARASEEEAAADRASRLAV
DIAAKLLDRLPDDARVAGFIDGLAEGLEALPEASRAVIGVDGAPVRVTAARALMPAEEEACRTRLS
QALGRPVTLAVTIDPALIAGLEMETPHAVVRNSFKADLDRVTAALTHHGT
SEQ ID NO: 24:
Nucleotide sequence of ATP synthase subunit c, sodium ion specific atpE_1
ATGACTGTCGAGATGGTCAGCATCTTCGCGGCGGCGCTCGCCGTCTCCTTCGGCGCCATCGG
GCCGGCCCTGGGCGAGGGCCGGGCGGTGGCCGCGGCCATGGACGCCATCGCCCGCCAGCC
GGAGGCGGCCGGAACCTTGTCGCGCACGCTCTTCGTCGGCCTCGCCATGATCGAGACCATGG
CGATCTACTGCCTGGTGATCGCGCTCCTGGTGCTCTTCGCCAATCCGTTCGTGAAGTGA
SEQ ID NO: 25:
Amino acid sequence of ATP synthase subunit c, sodium ion specific atpE_1
MTVEMVSIFAAALAVSFGAIGPALGEGRAVAAAMDAIARQPEAAGTLSRTLFVGLAMIETMAIYCL
VIALLVLFANPFVK
SEQ ID NO: 26:
Nucleotide sequence of ATP synthase subunit a atpB_1
ATGGGCTCGCCGCTGATCCTCGAACCCCTGTTCCATATCGGGCCCGTGCCCATCACCGCGCC
GGTGGTGGTCACCTGGCTCATCATGGCCGCCTTCATTGGGCTGGCGCGGCTCATCACCCGGA
AGCTTTCCACCGATCCCACCCGGACCCAGGCGGCGGTGGAAACGGTGCTGACCGCCATCGAT
TCCCAGATCGCCGACACCATGCAGGCCGATCCCGCGCCTTATCGCGCGCTCATCGGCACCAT
CTTCCTTTATGTGCTGGTGGCCAACTGGTCCTCGCTCATCCCGGGCATCGAGCCGCCCACGG
CGCATATCGAGACCGATGCGGCGCTCGCTTTCATCGTGTTCGCCGCCACCATCGGGTTCGGG
TTGAAGACAAGGGGTGTGAAGGGCTATCTCGCCACCTTCGCCGAACCCTCCTGGGTGATGAT
CCCGCTCAATGTGGTGGAGCAGATCACCCGGACCTTCTCGCTCATCGTGCGCCTGTTCGGCA
ACATCATGAGCGGGGTGTTCGTGGTCGGCATCATCCTGTCCCTCGCCGGGCTGCTGGTGCCC
ATCCCCCTCATGGCGCTCGATCTCCTGACCGGCGCCGTGCAGGCCTACATCTTCGCGGTGCT
GGCCTGCGTGTTCATCGGCGCGGCCATTGGCGAGGCGCCGGCAAAGCCCCAATCGAAGGAG
CCAGGGAAAACATCATGA
SEQ ID NO: 27:
Amino acid sequence of ATP synthase subunit a atpB_1
MGSPLILEPLFHIGPVPITAPVVVTWLIMAAFIGLARLITRKLSTDPTRTQAAVETVLTAIDSQIADT
MQADPAPYRALIGTIFLYVLVANWSSLIPGIEPPTAHIETDAALAFIVFAATIGFGLKTRGVKGYLAT
FAEPSWVMIPLNVVEQITRTFSLIVRLFGNIMSGVFVVGIILSLAGLLVPIPLMALDLLTGAVQAYIF
AVLACVFIGAAIGEAPAKPQSKEPGKTS
SEQ ID NO: 28:
Nucleotide sequence of ATP synthase epsilon chain atpC_1
GTGAGCGCGCCGCTGCACCTCACCATCACCACGCCGGCCGCCGTTCTGGTGGACCGTGCCGA
CATCGTGGCCCTGCGTGCCGAGGACGAGAGCGGCAGCTTCGGCATCCTGCCCGGCCATGCG
GATTTCCTGACCGTTCTGGAGGCCTGCGTGGTGCGCTTCAAGGATGGGGCCGACGGCGTGCA
TTATTGTGCTCTCAGTGGTGGCGTGCTGTCGGTCGAGGAGGGCCGGCGCATCGCCATCGCCT
GCCGTCAGGGCACGGTGAGCGACGACCTGGTCGCCCTGGAAGGGGCGGTGGACGCCATGC
GTTCGGCGGAGAGCGATGCCGACAAGCGGGCCCGGGTGGAGCAGATGCGCCTTCATGCCCA
CGCCGTGCGCCAGCTCCTGCACTATCTGCGGCCCGGCCGGGCCGGCGGCGTGGCGCCGGCC
GCCGCGCCGGAGGAGGGGCCGTCATGA
SEQ ID NO: 29:
Amino acid sequence of ATP synthase epsilon chain atpC_1
MSAPLHLTITTPAAVLVDRADIVALRAEDESGSFGILPGHADFLTVLEACVVRFKDGADGVHYCAL
SGGVLSVEEGRRIAIACRQGTVSDDLVALEGAVDAMRSAESDADKRARVEQMRLHAHAVRQLL
HYLRPGRAGGVAPAAAPEEGPS
SEQ ID NO: 30:
Nucleotide sequence of ATP synthase subunit beta atpD_1
ATGGCAGCGGCAGATGAGGAGGCGCAATCGGCCGCCGGCCCCGCCTCGGGCCGGGTGGTG
GCCGTGCGCGGCGCGGTGATCGACATCGCCTTTGCCCAGCCTCCGCTGCCGCCGCTGGACG
ACGCCCTTCTCATCACCGACGGCCGGGGCGGCACGGTGCTGGTGGAGGTGCAGAGCCATAT
GGATCGGCACACGGTGCGCGCCATCGCCCTTCAGGCCACCACCGGCCTCAGCCGGGGGCTG
GAGGCGGCGCGGGTGGGCGGGCCGGTGAAGGTGCCGGTGGGAGACCATGTGCTCGGCCGC
CTCCTGGATGTCACCGGCGCCATCGGCGACAAGGGCGGGCCGCTGCCGGCCGACGTGCCCA
CGCGGCCGATCCACCACGCGCCGCCATCCTTCGCCGCGCAGGGCGGCACGTCCGATCTGTTT
CGCACCGGCATCAAGGTCATCGACCTCCTGGCGCCCCTCGCCCAGGGCGGCAAGGCGGCCA
TGTTCGGCGGGGCCGGCGTGGGCAAGACCGTGCTGGTGATGGAGCTGATCCACGCCATGGT
GGCGAGCTACAAGGGCATCTCGGTGTTTGCCGGCGTGGGGGAGCGCTCCCGCGAGGGCCAC
GAGATGCTGCTGGACATGACCGATTCCGGCGTGCTCGACCGCACCGTTCTGGTCTATGGCCA
GATGAACGAGCCCCCCGGGGCCCGCTGGCGGGTGCCCATGACGGCGCTGACCATCGCCGAA
TATTTCCGCGACGAGAAGCACCAGAACGTCCTGCTGCTGATGGACAACATCTTCCGCTTCGTC
CAGGCGGGGGCGGAGGTCTCCGGCCTTTTGGGCCGTCCGCCCTCCCGGGTGGGATACCAGC
CGACGCTGGCGAGCGAGGTGGCGGCGCTCCAGGAACGCATCACCTCCGTGGGCGAGGCCTC
GGTGACCGCCATCGAGGCGGTCTACGTGCCGGCGGATGACTTCACCGATCCCGCCGTGACCA
CCATCGCCGCCCACGTGGATTCCATGGTGGTGCTCTCCCGCGCCATGGCGGCGGAGGGCAT
GTATCCGGCGGTGGACCCCATCTCCTCCTCGTCGGTGCTGCTCGACCCGCTCATCGTGGGGG
ACGAGCATGCGCGCGTCGCCAACGAGGTGCGCCGGACCATCGAGCATTATCGCGAGCTTCAG
GATGTGATCTCGCTGCTGGGCATGGAGGAATTGGGCACCGAGGATCGCCGCATCGTGGAGC
GGGCGCGCCGGCTCCAGCGCTTCCTCACCCAGCCCTTCACGGTCACCGAGGCCTTCACCGGC
GTGCCCGGCCGCTCGGTGGCCATCGCCGACACCATCGCCGGCTGCAGGATGATCCTGTCCG
GCGCCTGCGACGACTGGCAGGAAAGCGCCCTCTACATGGTGGGCACCATCGACGAGGCCCG
CCAGAAGGAGGAGGCCGCTCGCGCCAAGGCGGGGCAGGGCGCCCCGGCCGGGACGGCAGC
CGAGACGGCGGAGGCCGCCCCGTGA
SEQ ID NO: 31:
Amino acid sequence of ATP synthase subunit beta atpD_1
MAAADEEAQSAAGPASGRVVAVRGAVIDIAFAQPPLPPLDDALLITDGRGGTVLVEVQSHMDRH
TVRAIALQATTGLSRGLEAARVGGPVKVPVGDHVLGRLLDVTGAIGDKGGPLPADVPTRPIHHAP
PSFAAQGGTSDLFRTGIKVIDLLAPLAQGGKAAMFGGAGVGKTVLVMELIHAMVASYKGISVFAG
VGERSREGHEMLLDMTDSGVLDRTVLVYGQMNEPPGARWRVPMTALTIAEYFRDEKHQNVLLLM
DNIFRFVQAGAEVSGLLGRPPSRVGYQPTLASEVAALQERITSVGEASVTAIEAVYVPADDFTDPA
VTTIAAHVDSMVVLSRAMAAEGMYPAVDPISSSSVLLDPLIVGDEHARVANEVRRTIEHYRELQD
VISLLGMEELGTEDRRIVERARRLQRFLTQPFTVTEAFTGVPGRSVAIADTIAGCRMILSGACDDW
QESALYMVGTIDEARQKEEAARAKAGQGAPAGTAAETAEAAP
SEQ ID NO: 32:
Nucleotide sequence of ATP synthase subunit beta atpD_2
ATGGCGAACAAGGTCGGACGCATCACCCAGATCATCGGCGCCGTCGTCGACGTGCAGTTCGA
CGGGCATCTGCCGGCGATTCTCAACGCGATCGAGACCACCAACCAGGGCAACCGGCTGGTGC
TCGAAGTGGCTCAGCATCTCGGCGAGAACACCGTGCGCTGCATCGCCATGGATGCCACTGAA
GGCCTGGTGCGTGGCCAGGAGGTGGCCGACACCGATGCGCCCATCCAGGTGCCCGTGGGCG
CCGCCACCCTCGGCCGCATCATGAACGTGATCGGCGAGCCGGTGGACGAGCTGGGCCCCAT
CGAGGGCGAAGCGCTGCGCGGCATCCATCAGCCGGCCCCCTCCTATGCGGAGCAGGCCACG
GAAGCTGAGATCCTCGTCACCGGCATCAAGGTGGTGGATCTGCTGGCGCCCTATTCCAAGGG
CGGCAAGGTGGGCCTGTTCGGCGGCGCCGGCGTGGGCAAGACCGTGCTCATCATGGAGCTG
ATCAACAACGTGGCCAAGGCGCACGGCGGCTATTCCGTGTTCGCCGGCGTGGGTGAGCGCA
CCCGCGAGGGCAACGACCTCTACCACGAGATGATCGAGTCCAACGTGAACAAGGACCCGCAC
GAGAACAATGGCTCGGCGGCCGGTTCCAAGTGCGCCCTGGTCTATGGCCAGATGAACGAGCC
GCCCGGCGCCCGCGCCCGCGTGGCCCTCACCGGCCTCACCGTCGCCGAGCATTTCCGCGAC
CAGGGCCAGGACGTGCTGTTCTTCGTGGACAACATCTTCCGCTTCACCCAGGCGGGCTCCGA
GGTGTCGGCGCTTCTCGGCCGCATCCCCTCGGCGGTGGGCTACCAGCCGACGCTGGCCACC
GACATGGGCCAGCTGCAGGAGCGCATCACCACCACCACCAAGGGCTCCATCACCTCGGTGCA
GGCCATCTACGTGCCGGCGGACGATCTGACCGATCCGGCGCCGGCCGCCTCCTTCGCCCATC
TGGACGCCACCACGGTGCTGTCGCGCTCCATCGCGGAGAAGGGCATCTACCCGGCGGTGGA
TCCGCTGGACTCCACCTCGCGCATGCTGTCTCCCGCCATCCTCGGCGACGAGCACTACAACAC
CGCGCGCCAGGTGCAGCAGACCCTGCAGCGCTACAAGGCGCTCCAGGACATCATCGCCATCC
TGGGCATGGACGAACTCTCCGAAGAGGACAAGCTCACCGTGGCCCGCGCCCGCAAGATCGA
GCGCTTCCTCTCCCAGCCCTTCCACGTGGCCGAGGTGTTCACCGGTTCGCCCGGCAAGCTGG
TCGACCTCGCCGACACCATCAAGGGCTTCAAGGGCCTGGTGGACGGCAAGTACGACTACCTG
CCCGAGCAGGCCTTCTACATGGTGGGCACCATCGAAGAAGCCATCGAGAAGGGCAAGAAGCT
GGCGGCCGAGGCGGCCTGA
SEQ ID NO: 33:
Amino acid sequence of ATP synthase subunit beta atpD_2
MANKVGRITQIIGAVVDVQFDGHLPAILNAIETTNQGNRLVLEVAQHLGENTVRCIAMDATEGLV
RGQEVADTDAPIQVPVGAATLGRIMNVIGEPVDELGPIEGEALRGIHQPAPSYAEQATEAEILVTG
IKVVDLLAPYSKGGKVGLFGGAGVGKTVLIMELINNVAKAHGGYSVFAGVGERTREGNDLYHEMI
ESNVNKDPHENNGSAAGSKCALVYGQMNEPPGARARVALTGLTVAEHFRDQGQDVLFFVDNIFR
FTQAGSEVSALLGRIPSAVGYQPTLATDMGQLQERITTTTKGSITSVQAIYVPADDLTDPAPAASF
AHLDATTVLSRSIAEKGIYPAVDPLDSTSRMLSPAILGDEHYNTARQVQQTLQRYKALQDIIAILG
MDELSEEDKLTVARARKIERFLSQPFHVAEVFTGSPGKLVDLADTIKGFKGLVDGKYDYLPEQAFY
MVGTIEEAIEKGKKLAAEAA
SEQ ID NO: 34:
Nucleotide sequence of ATP synthase gamma chain atpG_2
ATGGCGAGTCTGAAGGACCTGAGAAACCGCATTGCCTCGGTGAAGGCGACGCAGAAGATCAC
CAAGGCGATGCAGATGGTCGCCGCGGCGAAGCTGCGTCGCGCCCAGGCGGCGGCTGAAGC
GGCCCGTCCCTATGCGGAACGCATGGAGACGGTGCTCGGAAATCTTGCCTCCGGCATGGTGG
TGGGCGCGCAGGCGCCTGTTCTCATGACCGGGACGGGCAAGAGCGACACCCACCTGCTGCT
GGTGTGCACCGGCGAGCGCGGCCTGTGCGGCGCCTTCAACTCGTCCATCGTGCGCTTCGCCC
GCGAGCGGGCGCAGCTGCTGCTGGCCGAGGGCAAGAAGGTGAAAATCCTGTGCGTGGGCCG
CAAGGGCCACGAGCAGCTGCGCCGCATCTACCCGGACAACATCATCGACGTGGTGGACCTGC
GCGCGGTGCGCAACATCGGCTTCAAGGAGGCCGACGCCATCGCCCGCAAGGTGCTGGCCCT
GCTCGATGAAGGCGCATTCGACGTCTGCACGCTCTTCTACTCCCACTTCAGGAGCGTGATCGC
CCAGGTGCCGACGGCCCAGCAGCTCATTCCGGCCACCTTCGACGAGCGGCCGGCCGTCGCC
GATGCGCCGGTCTATGAATATGAGCCGGAGGAGGAGGAGATCCTCGCCGAGCTGCTGCCGC
GCAACGTGGCGGTGCAGATCTTCAAGGCCCTCCTCGAGAACCAGGCTTCTTTCTATGGCTCCC
AGATGAGCGCCATGGACAACGCCACGCGCAATGCGGGCGAGATGATCAAGAAGCAGACGCT
CACCTACAACCGTACCCGCCAGGCCATGATCACGAAGGAACTCATCGAGATCATCTCCGGCG
CCGAGGCCGTCTGA
SEQ ID NO: 35:
Amino acid sequence of ATP synthase gamma chain atpG_2
MASLKDLRNRIASVKATQKITKAMQMVAAAKLRRAQAAAEAARPYAERMETVLGNLASGMVVGA
QAPVLMTGTGKSDTHLLLVCTGERGLCGAFNSSIVRFARERAQLLLAEGKKVKILCVGRKGHEQL
RRIYPDNIIDVVDLRAVRNIGFKEADAIARKVLALLDEGAFDVCTLFYSHFRSVIAQVPTAQQLIPA
TFDERPAVADAPVYEYEPEEEEILAELLPRNVAVQIFKALLENQASFYGSQMSAMDNATRNAGEMI
KKQTLTYNRTRQAMITKELIEIISGAEAV
SEQ ID NO: 36:
Nucleotide sequence of ATP synthase subunit alpha atpA_2
ATGGACATTCGAGCCGCTGAAATCTCTGCCATCCTGAAAGAGCAGATCCAGAATTTCGGCCAG
GAGGCGGAAGTCTCCGAGGTGGGTCAGGTTCTGTCCGTGGGTGACGGCATCGCGCGCGTCT
ACGGCCTCGACAACGTCCAGGCGGGCGAGATGGTCGAGTTCGAGAACGGCACGCGCGGCAT
GGCGCTGAACCTCGAGCTCGACAATGTCGGCATCGTGATCTTCGGTTCCGACCGCGAGATCA
AGGAAGGCCAGACCGTCAAGCGGACCGGCGCCATCGTGGACGCCCCCGTCGGCAAGGGCCT
GCTCGGCCGCGTCGTGGACGCTCTCGGCAACCCGATCGACGGCAAGGGCCCGATCATGTTCA
CCGAGCGTCGCCGGGTCGACGTGAAGGCGCCGGGCATCATCCCGCGCAAGTCGGTGCACGA
GCCCATGCAGACCGGCCTGAAGGCCATCGATGCGCTCATCCCCATCGGCCGCGGCCAGCGC
GAGCTCATCATCGGCGACCGCCAGACCGGCAAGACCGCCGTGGCGCTCGACTCGATCCTGAA
CCAGAAGCCCATCAACCAGGGCGACGACGAGAAGGCCAAGCTCTACTGCGTCTATGTCGCGG
TGGGCCAGAAGCGTTCCACTGTCGCGCAGTTCGTGAAGGTGCTCGAGGAGCACGGCGCGCT
GGAATATTCCATCGTCGTCGCCGCCACCGCCTCGGACGCGGCCCCCATGCAGTTCCTGGCGC
CGTTCACCGGCACCGCCATGGGCGAGTATTTCCGCGACAACGGCATGCACGCCCTCATCATC
CATGATGACCTGTCCAAGCAGGCCGTGGCCTACCGCCAGATGTCGCTGCTGCTGCGCCGCCC
GCCGGGCCGCGAGGCCTATCCCGGCGATGTGTTCTACCTGCACTCCCGCCTCTTGGAGCGCG
CCGCCAAGCTCAATGACGAGCACGGCGCCGGCTCGCTGACCGCCCTGCCGGTGATCGAGAC
CCAGGCCAACGACGTGTCGGCCTACATCCCGACCAACGTGATCTCCATCACCGACGGTCAGA
TCTTCCTTGAATCCGATCTGTTCTACCAGGGCATCCGCCCGGCGGTGAACGTGGGCCTGTCG
GTGTCGCGCGTGGGCTCTTCGGCCCAGATCAAGGCGATGAAGCAGGTGGCCGGCAAGATCA
AGGGCGAGCTCGCCCAGTATCGCGAGCTGGCGGCCTTCGCCCAGTTCGGTTCGGACCTGGA
CGCGGCCACCCAGAAGCTGCTGAACCGCGGCGCCCGCCTCACCGAGCTGCTGAAGCAGAGC
CAGTTCTCGCCCCTCAAGGTGGAGGAGCAGGTGGCGGTGATCTATGCCGGCACCAATGGCTA
TCTCGATCCGCTGCCGGTCTCCAAGGTGCGCGAGTTCGAGCAGGGTCTGCTCCTGTCGCTGC
GCTCGCAGCATCCGGAGATCCTGGACGCCATCCGCACGTCCAAGGAGCTTTCCAAGGACACC
GCCGAGAAGCTGACGAAGGCCATCGACGCCTTCGCCAAGAGCTTCTCCTGA
SEQ ID NO: 37:
Amino acid sequence of ATP synthase subunit alpha atpA_2
MDIRAAEISAILKEQIQNFGQEAEVSEVGQVLSVGDGIARVYGLDNVQAGEMVEFENGTRGMAL
NLELDNVGIVIFGSDREIKEGQTVKRTGAIVDAPVGKGLLGRVVDALGNPIDGKGPIMFTERRRV
DVKAPGIIPRKSVHEPMQTGLKAIDALIPIGRGQRELIIGDRQTGKTAVALDSILNQKPINQGDDE
KAKLYCVYVAVGQKRSTVAQFVKVLEEHGALEYSIVVAATASDAAPMQFLAPFTGTAMGEYFRDN
GMHALIIHDDLSKQAVAYRQMSLLLRRPPGREAYPGDVFYLHSRLLERAAKLNDEHGAGSLTALP
VIETQANDVSAYIPTNVISITDGQIFLESDLFYQGIRPAVNVGLSVSRVGSSAQIKAMKQVAGKIK
GELAQYRELAAFAQFGSDLDAATQKLLNRGARLTELLKQSQFSPLKVEEQVAVIYAGTNGYLDPLP
VSKVREFEQGLLLSLRSQHPEILDAIRTSKELSKDTAEKLTKAIDAFAKSFS
SEQ ID NO: 38:
Nucleotide sequence of ATP synthase subunit delta atpH
GTGGCGGAAACGATCGTGTCAGGCATGGCGGGACGCTATGCGACCGCGCTGTTCGAGCTGG
CGGACGAAGCCGGTGCCATCGATTCCGTCCAGGCGGATCTTGATCGCCTGTCCGGCCTTCTG
GCCGAGAGCGCGGATCTGGCGCGGCTGGTCAAGAGCCCGGTCTTCACCGCCGAGCAGCAGC
TCGGCGCGATGGCGGCCATTCTCGATCAAGCAGGCATTTCCGGCCTTGCGGGCAAATTCGTG
AAGCTGGTGGCGCAGAACCGCCGCCTGTTCGCACTGCCGCGCATGATTGCCGAATACGCCGT
CCTGGTGGCCCGGAAGAAGGGCGAGACCTCGGCGAGCGTGACCGTTGCCACCCCCCTGAGC
GATGAGCATCTGGCCACGCTCAAGGCGGCCCTGGCTGAAAAGACCGGCAAGGACGTGAAGC
TCGACGTCACCGTCGATCCGTCCATCCTCGGTGGTCTCATCGTGAAGCTCGGCTCGCGCATG
GTCGATGCTTCCCTGAAGACCAAACTCAATTCTATCCGGCATGCGATGAAAGAGGTCCGCTGA
SEQ ID NO: 39:
Amino acid sequence of ATP synthase subunit delta atpH
MAETIVSGMAGRYATALFELADEAGAIDSVQADLDRLSGLLAESADLARLVKSPVFTAEQQLGAM
AAILDQAGISGLAGKFVKLVAQNRRLFALPRMIAEYAVLVARKKGETSASVTVATPLSDEHLATLK
AALAEKTGKDVKLDVTVDPSILGGLIVKLGSRMVDASLKTKLNSIRHAMKEVR
SEQ ID NO: 40:
Nucleotide sequence of ATP synthase subunit b atpF_2
ATGACCGAAATGGAACTGGCTGAGCTCTGGGTCGCCATCGCCTTCCTGGTTTTCGTAGGCCTC
CTGATCTATGCGGGCGCCCACCGCGCCATCGTCTCCGCCCTGGATTCCCGCGGCTCGCGCAT
CGCCTCGGAACTGGAGGAGGCCCGTCGGCTCAAGGAAGAGGCCCAGAAGCTGGTGGCCGAA
TTCAAGCGCAAGCAGCGCGAGGCCGAGGCCGAGGCCGAATCCATCGTCACCGGCGCCAAGG
CCGAGGCCGAGCGCCTCGCCGCCGAGGCCAAGGCGAAGATCGAGGATTTCGTCACCCGCCG
CACCAAGATGGCCGAGGACAAGATCGCCCAGGCCGAGCATCAGGCTCTGGCGGACGTGAAG
TCCATCGCCGCCGAGGCGGCGGCCAAGGCGGCCGAGGTGATCCTCGGCGCCCAGGCCACCG
GCGCGGTGGCGGAGCGTCTGCTGTCGGGCGCCATCTCCGAGGTCAAGACCAAGCTCAACTG
A
SEQ ID NO: 41:
Amino acid sequence of ATP synthase subunit b atpF_2
MTEMELAELWVAIAFLVFVGLLIYAGAHRAIVSALDSRGSRIASELEEARRLKEEAQKLVAEFKRK
QREAEAEAESIVTGAKAEAERLAAEAKAKIEDFVTRRTKMAEDKIAQAEHQALADVKSIAAEAAA
KAAEVILGAQATGAVAERLLSGAISEVKTKLN
SEQ ID NO: 42:
Nucleotide sequence of ATP synthase subunit b′ atpG_3
ATGATGATTGCATGGAAGCGGACCTTCGCAGTCGTGACCTTCGGGGCCGCCCTGATGGCCAT
GCCCGTCGCGGGCGTGGTCGCAGCTGAGACTTCTCCCGCTCCGGCGGCAGTGGCGCAGGCC
GATCATGCGGTGCCCACCGAGGCGGCCGGCCAGGGCACCGCCGATGCGGCCCATGCCGCCG
CGCCGGGCGAGGCCGCCCATGGTGGCGCGGCCAAGCACGAAACCCATTTCCCGCCCTTCGA
CGGCACCACCTTCGCCTCCCAGTTGCTGTGGCTCGCCGTCACCTTCGGCCTGCTTTACTACCT
CATGAGCAAGGTCACGCTGCCGCGCATCGGCCGCATCCTGGAAGAGCGCCACGACCGCATC
GCCGATGATCTGGAGGAAGCCTCCAAGCATCGCGCCGAGAGCGAGGCCGCCCAGCGGGCCT
ATGAGAAGGCGCTGAGCGAGGCCCGCGCGAAGGCCCATTCCATCGCCGCGGAAACCCGCGA
CCGCCTTGCCGCCCACGCCGACACCAACCGCAAGGCGCTGGAGAGCGAGCTCACCGCCAAG
CTGCAGGCGGCCGAGGAGCGCATCGCCACCACCAAGAGCGAAGCCCTCACCCATGTGCGCG
GCATCGCGGTGGACGCCACCCAATCCATCGTCTCCACCCTCATCGGTGTCGCGCCCGCGGCG
GCCGACGTGGAAAAAGCGGTGGACGGCGCCCTGTCCCAGCACGGCCAGGCCTGA
SEQ ID NO: 43:
Amino acid sequence of ATP synthase subunit b′ atpG_3
MMIAWKRTFAVVTFGAALMAMPVAGVVAAETSPAPAAVAQADHAVPTEAAGQGTADAAHAAAP
GEAAHGGAAKHETHFPPFDGTTFASQLLWLAVTFGLLYYLMSKVTLPRIGRILEERHDRIADDLEE
ASKHRAESEAAQRAYEKALSEARAKAHSIAAETRDRLAAHADTNRKALESELTAKLQAAEERIATT
KSEALTHVRGIAVDATQSIVSTLIGVAPAAADVEKAVDGALSQHGQA
SEQ ID NO: 44:
Nucleotide sequence of ATP synthase subunit c atpE_2
ATGGAAGCGGAAGCTGGAAAGTTCATCGGTGCCGGCCTCGCCTGCCTCGGCATGGGTCTCGC
TGGCGTCGGCGTCGGTAACATCTTCGGTAACTTCCTCTCCGGCGCCCTGCGCAACCCGTCCG
CTGCCGACGGCCAGTTCGCCCGCGCCTTCATCGGCGCCGCCCTCGCGGAAGGTCTCGGCATC
TTCTCGCTGGTCGTTGCGCTCGTCCTGCTGTTCGTGGCCTGA
SEQ ID NO: 45:
Amino acid sequence of ATP synthase subunit c atpE_2
MEAEAGKFIGAGLACLGMGLAGVGVGNIFGNFLSGALRNPSAADGQFARAFIGAALAEGLGIFSL
VVALVLLFVA
SEQ ID NO: 46:
Nucleotide sequence of ATP synthase subunit a atpB_2
ATGACCGTCGATCCGATCCACCAGTTCGAGATCAAGCGCTACGTGGATCTGCTGAACGTCGG
CGGTGTCCAGTTCTCCTTCACCAACGCAACGGTGTTCATGATTGGCATCGTCCTGGTGATTTT
CTTCTTCCTGACTTTCGCGACACGCGGTCGCACCCTTGTGCCGGGCCGGATGCAGTCGGCGG
CGGAGCTGAGCTACGAGTTCATCGCCAAGATGGTGCGCGACGCGGCCGGCAGCGAGGGAAT
GGTGTTCTTTCCCTTCGTCTTCTCGCTCTTCATGTTCGTGCTGGTGGCGAACGTATTGGGGCT
CATCCCCTACACCTTCACGGTGACCGCCCACCTCATCGTCACCGCCGCCCTGGCGGCGACGG
TGATCCTCACCGTCATCATCTACGGCTTCGTGCGGCACGGCACCCACTTCCTGCACCTGTTCG
TGCCGTCGGGCGTGCCGGGCTTCCTCCTGCCCTTCCTCGTGGTGATCGAGGTGGTGTCGTTC
CTGTCGCGGCCCATCAGCCTCTCGCTGCGTCTGTTCGCCAACATGCTGGCGGGCCACATCGC
CCTCAAGGTGTTCGCCTTCTTCGTCGTGGGACTGGCCTCGGCCGGCGCGATCGGCTGGTTCG
GCGCCACCCTGCCCTTCTTCATGATCGTGGCGCTCACCGCGCTGGAGCTGCTGGTGGCGGTG
CTGCAGGCCTACGTGTTCGCGGTGCTGACCTCGATCTACCTCAACGACGCCATCCATCCCGGC
CACTGA
SEQ ID NO: 47:
Amino acid sequence of ATP synthase subunit a atpB_2
MTVDPIHQFEIKRYVDLLNVGGVQFSFTNATVFMIGIVLVIFFFLTFATRGRTLVPGRMQSAAELSY
EFIAKMVRDAAGSEGMVFFPFVFSLFMFVLVANVLGLIPYTFTVTAHLIVTAALAATVILTVIIYGFV
RHGTHFLHLFVPSGVPGFLLPFLVVIEVVSFLSRPISLSLRLFANMLAGHIALKVFAFFVVGLASAGA
IGWFGATLPFFMIVALTALELLVAVLQAYVFAVLTSIYLNDAIHPGH
SEQ ID NO: 48:
Nucleotide sequence of ATP synthase protein I atpI
ATGTCCGAGCCGAATGATCCATCCCGCAGGGACGGTGCGAAGGCGAAAGACGAGACGCAGG
ACTCCCGGCCCGGTGAGGCGGATCTTGCTCGGCGCCTCGATGCGCTCGGCACCTCCATCGGT
CAGGTCAAGTCCAGAAGCGGGGAGCCCGCGGCGACGCCGCGCAAGGACACCTCCTCGGCCT
CCGGCGCGGCCCTGGCGTTTCGGCTGGGCGCCGAGTTTGTTTCAGGCGTGCTGGTGGGCTC
GCTCATCGGCTACGGGTTGGATTATGCGTTTGCGATTTCGCCCTGGGGGCTGATCGCCTTCAC
GCTGATCGGCTTTGCCGCCGGCGTCCTGAACATGCTGCGCGTGGCGAACAGCGATGCCAAGC
GCCACAGCGCGGACAGGTGA
SEQ ID NO: 49:
Amino acid sequence of ATP synthase protein I atpI
MSEPNDPSRRDGAKAKDETQDSRPGEADLARRLDALGTSIGQVKSRSGEPAATPRKDTSSASG
AALAFRLGAEFVSGVLVGSLIGYGLDYAFAISPWGLIAFTLIGFAAGVLNMLRVANSDAKRHSADR

In another embodiment, the variant chemoautotrophic bacterial strain used in the process of the aspects of the disclosed embodiments comprises a gene encoding a nitrogenase molybdenum-iron protein alpha chain nifD_1 having the sequence set forth in SEQ ID NO:51 or a sequence having more than 60% identity, e.g. more than 70% identity, such as more than 92% identity, such as more than 95% identity, e.g. more than 96% identity, such as more than 97% identity, e.g. more than 98%, such as more than 99% sequence identity to the sequence set forth in SEQ ID NO:51.

In another embodiment, the variant chemoautotrophic bacterial strain used in the process of the aspects of the disclosed embodiments comprises a gene encoding nitrogenase molybdenum-iron protein alpha chain nifD_2 having the sequence set forth in SEQ ID NO:53 or a sequence having more than 60% identity, e.g. more than 98%, such as more than 99% sequence identity to the sequence set forth in SEQ ID NO:53.

In another embodiment, the variant chemoautotrophic bacterial strain used in the process of the aspects of the disclosed embodiments comprises a gene encoding a nitrogenase molybdenum-iron protein beta chain nifK_1 having the sequence set forth in SEQ ID NO:55 or a sequence having more than 87% identity, e.g. more than 90% identity, such as more than 95% identity, e.g. more than 96% identity, such as more than 97% identity, e.g. more than 98%, such as more than 99% sequence identity to the sequence set forth in SEQ ID NO:55.

In another embodiment, the variant chemoautotrophic bacterial strain used in the process of the aspects of the disclosed embodiments comprises a gene encoding a nitrogenase molybdenum-iron protein beta chain nifK_2 having the sequence set forth in SEQ ID NO:57 or a sequence having more than 95% identity, e.g. more than 96% identity, such as more than 97% identity, e.g. more than 98%, such as more than 99% sequence identity to the sequence set forth in SEQ ID NO:57.

In another embodiment, the variant chemoautotrophic bacterial strain used in the process of the aspects of the disclosed embodiments comprises a gene encoding a nitrogenase iron protein nifH having the sequence set forth in SEQ ID NO:59 or a sequence having more than 98.5% sequence identity to the sequence set forth in SEQ ID NO:59.

SEQ ID NO: 50:
Nucleotide sequence of Nitrogenase molybdenum-iron protein alpha chain nifD_1
ATGAGTTCGCTCTCCGCCACTATTCAACAGGTCTTCAACGAGCCGGGCTGCGCGAAGAACCA
GAATAAGTCCGAGGCGGAGAAGAAGAAGGGCTGCACCAAGCAGCTGCAACCCGGCGGAGCG
GCCGGCGGCTGCGCGTTCGACGGCGCGAAGATCGCGCTCCAGCCCTTGACCGACGTCGCCC
ACCTGGTGCACGGCCCCATCGCCTGCGAAGGCAATTCCTGGGACAATCGTGGCGCCAAGTCC
TCCGGCTCGAACATCTGGCGCACCGGCTTCACCACGGACATCAACGAAACCGACGTGGTGTT
CGGCGGCGAGAAGCGTCTGTTCAAGTCCATCAAGGAAATCATCGAGAAGTACGACCCGCCGG
CCGTCTTCGTCTATCAGACCTGCGTCCCCGCCATGATCGGCGACGACATCGACGCGGTGTGC
AAGGCGGCCAGGGAGAAGTTCGGAAAGCCGGTGATCCCGATCAATTCCCCCGGCTTCGTGG
GGCCGAAGAATCTCGGCAACAAGCTCGCCGGCGAGGCGCTCCTCGACCATGTGATCGGCACC
GAGGAGCCCGATTACACGACGGCCTACGACATCAACATCATCGGCGAATACAATCTCTCCGG
CGAGTTGTGGCAGGTGAAGCCGCTGCTGGACGAGCTGGGCATCCGCATCCTCGCCTGCATCT
CCGGCGACGGGAAGTACAAGGATGTGGCGTCCTCCCACCGCGCCAAGGCGGCGATGATGGT
GTGCTCCAAGGCCATGATCAACGTGGCCCGCAAGATGGAGGAGCGCTACGACATCCCCTTCT
TCGAAGGCTCCTTCTACGGCATCGAGGATAGCTCCGATTCCCTGCGCGAGATTGCGCGCATG
CTCATCGAGAAGGGCGCCGATCCGGAGCTGATGGACCGCACCGAGGCGCTGATTGAGCGGG
AAGAGAAGAAGGCGTGGGACGCCATCGCCGCCTACAAGCCCCGCTTCAAGGACAAGAAGGT
GCTGCTCATCACCGGCGGCGTGAAATCCTGGTCGGTGGTGGCAGCGCTCCAGGAAGCCGGC
CTCGAACTGGTGGGCACCTCGGTGAAGAAGTCCACCAAGGAGGACAAGGAGCGCATCAAGG
AACTGATGGGCCAGGACGCCCACATGATCGACGACATGACGCCCCGCGAAATGTACAAGATG
CTGAAGGACGCCAAGGCGGACATCATGCTCTCGGGCGGGCGCTCGCAATTCATCGCGCTCAA
GGCCGCCATGCCCTGGCTCGACATCAACCAGGAGCGCCACCACGCCTATATGGGCTATGTGG
GCATGGTGAAGCTGGTCGAGGAGATCGACAAGGCGCTCTACAATCCCGTGTGGGAACAGGT
GCGCAAGCCCGCCCCGTGGGAAAATCCGGAAGACACCTGGCAGGCCCGTGCGCTCGCCGAA
ATGGAGGCGGAGGCCGCCGCGCTCGCCGCCGATCCGGTGCGCGCGGAAGAGGTGCGCCGG
TCCAAGAAGATCTGCAATTGCAAGAGCGTCGACCTCGGAACCATTGAGGACGCCATCAAGGC
TCACGCGCTGACCACCGTGGAGGGTGTGCGAGAGCACACCAATGCCTCGGGAGGCTGCGGA
GCCTGCAGCGGGCGGATCGAGGAGATCTTCGAGGCCGTGGGCGTTGTCGCCGCCCCGCCTC
CCGCGGAGGCCGCCCCGTCTCCGCAGGAGATCGCGCCCGATCCGCTCGCTGCGGAGGAAAA
GCGCCGCGCCAAGAAGGCCTGCGGCTGCAAGGAGGTAGCGGTCGGCACCATTGAGGATGCC
ATCCGCGCCAAGGGTCTGCGAAACATCGCGGAGGTGCGTGCGGCCACCGATGCCAACACCG
GCTGCGGCAATTGCCAGGAGCGGGTGGAGGGCATCCTCGACCGGGTTCTCGCCGAGGCGGC
CTCAGAACTCCAGGCGGCGGAATAG
SEQ ID NO: 51:
Amino acid sequence of Nitrogenase molybdenum-iron protein alpha chain nifD_1
MSSLSATIQQVFNEPGCAKNQNKSEAEKKKGCTKQLQPGGAAGGCAFDGAKIALQPLTDVAHLV
HGPIACEGNSWDNRGAKSSGSNIWRTGFTTDINETDVVFGGEKRLFKSIKEIIEKYDPPAVFVYQ
TCVPAMIGDDIDAVCKAAREKFGKPVIPINSPGFVGPKNLGNKLAGEALLDHVIGTEEPDYTTAYD
INIIGEYNLSGELWQVKPLLDELGIRILACISGDGKYKDVASSHRAKAAMMVCSKAMINVARKME
ERYDIPFFEGSFYGIEDSSDSLREIARMLIEKGADPELMDRTEALIEREEKKAWDAIAAYKPRFKDK
KVLLITGGVKSWSVVAALQEAGLELVGTSVKKSTKEDKERIKELMGQDAHMIDDMTPREMYKML
KDAKADIMLSGGRSQFIALKAAMPWLDINQERHHAYMGYVGMVKLVEEIDKALYNPVWEQVRKP
APWENPEDTWQARALAEMEAEAAALAADPVRAEEVRRSKKICNCKSVDLGTIEDAIKAHALTTVE
GVREHTNASGGCGACSGRIEEIFEAVGVVAAPPPAEAAPSPQEIAPDPLAAEEKRRAKKACGCKE
VAVGTIEDAIRAKGLRNIAEVRAATDANTGCGNCQERVEGILDRVLAEAASELQAAE
SEQ ID NO: 52:
Nucleotide sequence of Nitrogenase molybdenum-iron protein alpha chain nifD_2
ATGAGTGTCGCACAGTCCCAGAGCGTCGCCGAGATCAAGGCGCGCAACAAGGAACTCATCGA
AGAGGTCCTCAAGGTCTATCCCGAGAAGACCGCCAAGCGCCGCGCCAAGCACCTGAACGTCC
ACGAAGCCGGCAAGTCCGACTGCGGCGTGAAGTCCAACATCAAGTCCATCCCGGGCGTGATG
ACCATCCGCGGTTGCGCTTATGCCGGCTCCAAGGGTGTGGTGTGGGGTCCCATCAAGGACAT
GATCCACATCTCCCACGGCCCGGTGGGCTGCGGCCAGTATAGCTGGGCCGCCCGCCGCAACT
ACTATATCGGCACGACCGGCATCGACACCTTCGTGACGATGCAGTTCACCTCCGACTTCCAGG
AGAAGGACATCGTCTTCGGCGGCGACAAGAAGCTCGCCAAGATCATGGACGAGATCCAGGAG
CTGTTCCCGCTGAACAACGGCATCACCGTTCAGTCCGAGTGCCCCATCGGCCTCATCGGCGA
CGACATCGAGGCCGTCTCCAAGCAGAAGTCCAAGGAGTATGAGGGCAAGACCATCGTGCCGG
TGCGCTGCGAGGGCTTCCGCGGCGTGTCCCAGTCCCTGGGCCACCACATCGCCAACGACGCC
ATCCGCGATTGGGTGTTCGACAAGATCGCGCCCGACGCCGAGCCGCGCTTTGAGCCGACCCC
GTACGACGTCGCCATCATCGGCGACTACAATATCGGTGGTGACGCCTGGTCGTCCCGTATCCT
CCTGGAGGAGATGGGCCTGCGCGTGATCGCCCAGTGGTCCGGCGACGGTTCGCTCGCTGAG
CTGGAGGCCACCCCGAAGGCCAAGCTCAACGTGCTGCACTGCTACCGCTCCATGAACTACAT
CTCGCGCCACATGGAAGAGAAGTACGGTATCCCGTGGTGCGAGTACAACTTCTTCGGTCCTTC
CAAGATCGCCGAGTCCCTGCGCAAGATCGCCAGCTACTTCGACGACAAGATCAAGGAAGGCG
CGGAGCGCGTCATCGCCAAGTATCAGCCGCTCATGGATGCGGTGATCGCGAAGTATCGTCCC
CGCCTCGAGGGCAAGACCGTGATGCTGTACGTGGGCGGCCTGCGTCCCCGTCACGTCATCG
GCGCCTACGAGGACCTGGGCATGGAAGTGGTCGGCACGGGCTACGAGTTCGCCCATAACGA
CGACTACCAGCGCACCGCCCAGCACTACGTCAAGGATGGCACCATCATCTATGACGACGTGA
CCGGCTACGAGTTCGAGAAGTTCGTCGAGAAGATCCAGCCGGACCTGGTCGGTTCGGGCATC
AAGGAAAAGTACGTCTTCCAGAAGATGGGCGTGCCGTTCCGCCAGATGCACTCCTGGGACTA
CTCGGGCCCGTACCACGGCTATGACGGCTTCGCGATCTTCGCGCGCGACATGGACATGGCCA
TCAACAGCCCCGTGTGGAAGATGACCCAGGCTCCGTGGAAGAGCGTCCCCAAGCCGACGATG
CTCGCGGCTGAATGA
SEQ ID NO: 53:
Amino acid sequence of Nitrogenase molybdenum-iron protein alpha chain nifD_2
MSVAQSQSVAEIKARNKELIEEVLKVYPEKTAKRRAKHLNVHEAGKSDCGVKSNIKSIPGVMTIR
GCAYAGSKGVVWGPIKDMIHISHGPVGCGQYSWAARRNYYIGTTGIDTFVTMQFTSDFQEKDIV
FGGDKKLAKIMDEIQELFPLNNGITVQSECPIGLIGDDIEAVSKQKSKEYEGKTIVPVRCEGFRGV
SQSLGHHIANDAIRDWVFDKIAPDAEPRFEPTPYDVAIIGDYNIGGDAWSSRILLEEMGLRVIAQ
WSGDGSLAELEATPKAKLNVLHCYRSMNYISRHMEEKYGIPWCEYNFFGPSKIAESLRKIASYFD
DKIKEGAERVIAKYQPLMDAVIAKYRPRLEGKTVMLYVGGLRPRHVIGAYEDLGMEVVGTGYEFA
HNDDYQRTAQHYVKDGTIIYDDVTGYEFEKFVEKIQPDLVGSGIKEKYVFQKMGVPFRQMHSWD
YSGPYHGYDGFAIFARDMDMAINSPVWKMTQAPWKSVPKPTMLAAE
SEQ ID NO: 54:
Nucleotide sequence of Nitrogenase molybdenum-iron protein beta chain nifK_1
ATGGCCACCGTTTCCGTCTCCAAGAAGGCCTGCGCGGTCAACCCCCTCAAGATGAGCCAGCC
GGTGGGCGGCGCGCTCGCCTTCATGGGCGTGCGCAAGGCCATGCCGCTGCTGCACGGCTCG
CAGGGCTGCACCTCCTTCGGCCTGGTGCTGTTCGTGCGCCACTTCAAGGAAGCCATCCCCAT
GCAGACCACCGCCATGAGCGAGGTGGCGACGGTTCTGGGCGGCCTTGAGAATGTGGAGCAG
GCCATTCTCAACATCTACAATCGCACCAAGCCGGAGATCATCGGCATCTGCTCCACCGGCGTC
ACCGAGACCAAGGGCGATGATGTCGACGGCTACATCAAGCTGATCCGGGACAAGTATCCCCA
GCTGGCCGACTTCCCGCTGGTCTATGTCTCCACCCCCGATTTCAAGGACGCCTTCCAGGACG
GTTGGGAGAAGACCGTGGCGAAGATGGTGGAGGCGCTGGTGAAGCCCGCCGCCGACAAGCA
GAAGGACAAGACCCGCGTCAACGTCCTGCCCGGCTGCCACCTCACGCCCGGCGATCTGGATG
AGATGCGGACCATCTTCGAGGATTTCGGGCTCACACCCTATTTCCTGCCGGATCTGGCCGGCT
CGCTGGATGGGCATATCCCCGAGGACTTCTCGCCCACCACCATCGGCGGCATCGGCATCGAT
GAGATCGCCACCATGGGCGAGGCGGCCCACACCATCTGCATCGGCGCGCAGATGCGCCGGG
CGGGCGAGGCCATGGAGAAGAAGACCGGCATTCCCTTCAAGCTGTTCGAGCGCCTGTGCGG
CCTGGAGGCGAACGACGCCTTCATCATGCACCTGTCGCAGATCTCCGGCCGGCCGGTGCCGG
TGAAGTATCGCCGGCAGCGGGGCCAGCTGGTGGATGCCATGCTGGACGGCCACTTCCATCTG
GGCGGTCGCAAGGTGGCCATGGGGGCGGAGCCGGACCTGCTCTACGACGTGGGCTCCTTCC
TGCACGAGATGGGCGCCCACATCCTTTCCGCGGTCACCACCACCCAGTCGCCGGTGCTGGCG
CGCCTGCCTGCCGAGGAGGTGCTTATCGGCGACCTGGAGGATCTGGAGACCCAGGCGAAGG
CGCGCGGATGCGATCTCCTGCTCACCCATTCCCATGGGCGCCAGGCGGCGGAGCGCCTCCAC
ATCCCCTTCTACCGGATCGGCATTCCCATGTTTGACCGGCTGGGGGCGGGGCATCTGTTGTC
GGTGGGCTATCGCGGCACCCGCGACCTCATCTTCCATCTCGCCAACCTTGTGATCGCCGACCA
CGAGGAAAATCACGAGCCGACGCCCGACACCTGGGCCACCGGCCATGGCGAGCATGCCGCC
GCCCCCACTTCCCATTGA
SEQ ID NO: 55:
Amino acid sequence of Nitrogenase molybdenum-iron protein beta chain nifK_1
MATVSVSKKACAVNPLKMSQPVGGALAFMGVRKAMPLLHGSQGCTSFGLVLFVRHFKEAIPMQT
TAMSEVATVLGGLENVEQAILNIYNRTKPEIIGICSTGVTETKGDDVDGYIKLIRDKYPQLADFPLV
YVSTPDFKDAFQDGWEKTVAKMVEALVKPAADKQKDKTRVNVLPGCHLTPGDLDEMRTIFEDFG
LTPYFLPDLAGSLDGHIPEDFSPTTIGGIGIDEIATMGEAAHTICIGAQMRRAGEAMEKKTGIPFKL
FERLCGLEANDAFIMHLSQISGRPVPVKYRRQRGQLVDAMLDGHFHLGGRKVAMGAEPDLLYDV
GSFLHEMGAHILSAVTTTQSPVLARLPAEEVLIGDLEDLETQAKARGCDLLLTHSHGRQAAERLHI
PFYRIGIPMFDRLGAGHLLSVGYRGTRDLIFHLANLVIADHEENHEPTPDTWATGHGEHAAAPTS
H
SEQ ID NO: 56:
Nucleotide sequence of Nitrogenase molybdenum-iron protein beta chain nifK_2
ATGCCACAAAATGCTGACAATGTGCTCGATCACTTCGAGCTCTTCCGTGGTCCCGAATACCAG
CAGATGCTGGCCAATAAGAAAAAGATGTTCGAGAACCCCCGCGATCCGGCCGAAGTCGAGCG
CGTGCGGGAATGGGCGAAGACTCCTGAATACAAGGAGCTGAACTTCGCCCGCGAGGCGCTC
ACCGTGAATCCGGCCAAGGCTTGTCAGCCGCTGGGCGCGGTGTTCGTCGCCGTCGGCTTCGA
GAGCACGATCCCCTTCGTGCACGGCTCGCAGGGTTGCGTCGCGTATTACCGCTCGCACCTCT
CCCGCCACTTCAAGGAGCCGTCCTCCTGCGTCTCCTCGTCCATGACCGAGGATGCGGCGGTG
TTCGGCGGCCTCAACAACATGATTGACGGCCTCGCCAACACCTACAACATGTACAAGCCGAAG
ATGATCGCCGTCTCCACCACCTGCATGGCGGAAGTCATCGGCGACGATCTGAACGCCTTCATC
AAGACCGCGAAGGAAAAGGGCTCGGTTCCGGCCGAATACGACGTGCCCTTCGCCCACACCCC
GGCGTTCGTCGGCAGCCATGTCACCGGCTACGACAATGCGCTCAAGGGCATCCTCGAGCACT
TCTGGGACGGCAAGGCCGGCACCGCGCCGAAGCTGGAGCGCGTTCCCAACGAGAAGATCAA
CTTCATCGGCGGCTTCGACGGCTACACCGTCGGCAACACTCGCGAAGTGAAGCGCATCTTCG
AGGCGTTCGGCGCCGATTACACCATCCTCGCCGACAATTCCGAAGTGTTCGACACCCCGACC
GACGGCGAGTTCCGCATGTATGACGGCGGCACGACCCTGGAGGACGCGGCGAACGCGGTGC
ACGCCAAGGCCACCATCTCCATGCAGGAATACTGCACGGAGAAGACCCTGCCCATGATCGCC
GGTCATGGCCAGGACGTGGTCGCCCTCAACCACCCCGTGGGCGTGGGCGGCACCGACAAGT
TCCTCATGGAGATCGCCCGCCTCACCGGCAAGGAGATCCCCGAGGAGCTGACCCGCGAGCG
CGGCCGTCTCGTGGACGCTATCGCGGACTCTTCCGCGCACATCCACGGCAAGAAGTTCGCCA
TCTACGGCGATCCGGATCTGTGCCTGGGCCTCGCCGCGTTCCTGCTGGAGCTGGGCGCCGAG
CCGACCCATGTGCTGGCCACCAACGGCACCAAGAAGTGGGCCGAGAAGGTTCAGGAACTGTT
CGACTCTTCGCCGTTCGGCGCCAACTGCAAGGTCTATCCCGGCAAGGACCTGTGGCACATGC
GCTCGCTCCTGTTCGTGGAGCCGGTGGATTTCATCATCGGCAACACCTACGGCAAGTATCTCG
AGCGCGACACGGGCACCCCGCTGATCCGTATCGGCTTCCCGGTGTTCGACCGTCACCACCAC
CACCGCCGTCCGGTGTGGGGCTATCAGGGCGGCATGAACGTCCTGATCACGATCCTCGACAA
GATCTTTGACGAGATCGACCGCAACACCAACGTGCCGGCCAAGACCGACTACTCGTTCGACAT
CATTCGTTGA
SEQ ID NO: 57:
Amino acid sequence of Nitrogenase molybdenum-iron protein beta chain nifK_2
MPQNADNVLDHFELFRGPEYQQMLANKKKMFENPRDPAEVERVREWAKTPEYKELNFAREALTV
NPAKACQPLGAVFVAVGFESTIPFVHGSQGCVAYYRSHLSRHFKEPSSCVSSSMTEDAAVFGGLN
NMIDGLANTYNMYKPKMIAVSTTCMAEVIGDDLNAFIKTAKEKGSVPAEYDVPFAHTPAFVGSHV
TGYDNALKGILEHFWDGKAGTAPKLERVPNEKINFIGGFDGYTVGNTREVKRIFEAFGADYTILAD
NSEVFDTPTDGEFRMYDGGTTLEDAANAVHAKATISMQEYCTEKTLPMIAGHGQDVVALNHPVG
VGGTDKFLMEIARLTGKEIPEELTRERGRLVDAIADSSAHIHGKKFAIYGDPDLCLGLAAFLLELGA
EPTHVLATNGTKKWAEKVQELFDSSPFGANCKVYPGKDLWHMRSLLFVEPVDFIIGNTYGKYLER
DTGTPLIRIGFPVFDRHHHHRRPVWGYQGGMNVLITILDKIFDEIDRNTNVPAKTDYSFDIIR
SEQ ID NO: 58:
Nucleotide sequence of Nitrogenase iron protein nifH
GTGGAGTCCGGTGGTCCTGAGCCGGGCGTGGGCTGCGCCGGCCGCGGCGTGATCACCTCCA
TCAACTTCCTGGAGGAGAACGGCGCCTACGAGGACATCGACTATGTGTCCTACGACGTGCTG
GGCGACGTGGTGTGCGGCGGCTTCGCCATGCCCATCCGCGAGAACAAGGCGCAGGAAATCT
ACATCGTGATGTCCGGCGAGATGATGGCCATGTATGCGGCCAACAACATCTCCAAGGGCATC
CTGAAGTATGCCAATTCCGGCGGCGTGCGCCTGGGCGGGCTGGTCTGCAACGAGCGCCAGA
CCGACAAGGAGCTGGAGCTGGCGGAGGCTCTGGCGAAGAAGCTCGGCACCGAGCTGATCTA
CTTCGTGCCGCGCGACAACATCGTGCAGCATGCCGAGCTGCGCCGCATGACAGTGATCGAGT
ATGCGCCCGATTCCGCCCAGGCCCAGCACTACCGGAACCTGGCCGAGAAGGTGCACGCCAAC
AAGGGCAACGGCATCATCCCGACCCCGATCACCATGGACGAGCTGGAAGACATGCTCATGGA
GCACGGCATCATGAAGGCCGTGGACGAGAGCCAGATCGGCAAGACCGCCGCCGAGCTCGCC
GTCTGA
SEQ ID NO: 59:
Amino acid sequence of Nitrogenase iron protein nifH
MESGGPEPGVGCAGRGVITSINFLEENGAYEDIDYVSYDVLGDVVCGGFAMPIRENKAQEIYIVM
SGEMMAMYAANNISKGILKYANSGGVRLGGLVCNERQTDKELELAEALAKKLGTELIYFVPRDNI
VQHAELRRMTVIEYAPDSAQAQHYRNLAEKVHANKGNGIIPTPITMDELEDMLMEHGIMKAVDES
QIGKTAAELAV

### Genetic Modification of VTT-E-193585 and Variants Thereof, Such as Strain VTT-E-213595

As described above, in a further main aspect, the aspects of the disclosed embodiments relate to general methods for genetic modification of bacterial strain VTT-E-193585 or variants thereof and to genetically-modified variants of strain VTT-E-193585. These methods are exemplified in Example 5 herein.

Accordingly, in one aspect, the aspects of the disclosed embodiments relate to a method for genetic modification of bacterial strain VTT-E-193585 comprising the steps of:
a) providing bacteria of strain VTT-E-193585 or a genetically-modified or mutated strain generated using bacterial strain VTT-E-193585, such as strain VTT-E-213595,
b) introducing a nucleic acid construct into said bacteria, wherein said nucleic acid construct comprises:
   i) sequences encoding a selectable marker,
   ii) optionally further sequences to be integrated into the bacterial genome,
   iii) flanking sequences allowing homologous recombination with the bacterial genome, and,
c) selecting a genetically-modified strain wherein said nucleic acid construct has been integrated into the bacterial genome, on the basis of the selectable marker.

In one embodiment, the nucleic acid construct is a plasmid.

In some embodiments, the selectable marker is a gene providing antibiotic resistance, such as kanamycin or tetracycline resistance and step c) is carried out by growing the bacteria in the presence of antibiotics.

In another embodiment, the selectable marker is a gene encoding a fluorescent protein and step c) is carried out on the basis of fluorescence.

The flanking sequences are typically fully identical to sequences in the bacterial genome to allow for site-specific integration of the nucleic acid construct.

In one embodiment, the method is used to disrupt a gene by insertion. Thus, the flanking sequences are chosen such that upon integration of the nucleic acid construct, an endogenous gene in the bacterial genome has been disrupted and thus inactivated.

In another embodiment, the method is used to insert a gene, such as a heterologous gene or a mutated gene, or multiple copies of a gene into the bacterial genome.

In a further aspect, the aspects of the disclosed embodiments relate to a variant of bacterial strain VTT-E-193585 comprising a genetic modification wherein said genetic modification comprises the disruption of a bacterial gene with a selectable marker providing antibiotic resistance, such as kanamycin or tetracycline resistance. Such variants are exemplified herein, for instance in Example 5. The aspects of the disclosed embodiments also relate to a culture comprising such a variant, and to a process for the production of biomass, said process comprising culturing such variants. The process may have any of the further features described herein above.

### Downstream Processing

In one embodiment, the process of the aspects of the disclosed embodiment comprise the further step of harvesting biomass produced during the culture. Biomass can e.g. be harvested by sedimentation (settling based on gravity), filtration, centrifugation or flocculation. Flocculation may require the addition of a flocculation agent. Centrifugation may e.g. be carried out using a continuous flow centrifuge.

In one embodiment, the harvested biomass is subsequently dried. Drying can e.g. be performed using well known methods, including centrifugation, drum drying, evaporation, freeze drying, heating, spray drying, vacuum drying and/or vacuum filtration. The dried biomass may subsequently be used in a product, e.g. a food or feed product or feed or food ingredient.

In another embodiment, the cells of the harvested biomass are lysed. The lysate may in some embodiments be separated into insoluble and soluble fractions, either or both of which may subsequently be concentrated or dried, and subsequently be used in a product, e.g. a food or a feed product.

In one embodiment, biomass is harvested and proteins are isolated from said biomass, resulting in a protein fraction and a fraction comprising non-protein components. Thus, in one embodiment, the process is for the production of protein and comprises a step of culturing strain VTT-E-213595 thereof, followed by a step of harvesting biomass and a further step of isolating proteins from said biomass. In another embodiment, the process is for the production of protein and comprises culturing a variant chemoautotrophic bacterial strain, for example of the genus Xanthobacter, in continuous culture with hydrogen as energy source and an inorganic carbon source, wherein the inorganic carbon source comprises carbon dioxide, followed by a step of harvesting biomass and a further step of isolating proteins from said biomass. Depending on the method of protein isolation, the resulting fractions may be more pure or less pure. Thus, the term “protein fraction” means a fraction enriched in proteins. The protein fraction may still comprise significant amounts of other components and also significant amounts of protein may end up in the “fraction comprising non-protein components”.

Isolation of proteins may be performed using any suitable method. For example, in one embodiment, proteins are isolated by breaking cells mechanically and separating protein from cell debris through one or more filtration steps, e.g. successive filtration through multiple filters with decreasing pore size. Mechanical breaking may be carried out using any suitable method, e.g. ball milling, sonication, homogenization, high pressure homogenization, mechanical shearing, etc. The resulting filtered protein fraction will be enriched in proteins, but also still contain other smaller components. Protein may optionally be further purified from this fraction using any suitable method.

In another embodiment, a protein fraction is isolated by performing ethanol extraction followed by one or more filtration steps. Such methods are e.g. known from the preparation of soy bean proteins (see e.g. Chapter 5 “Soybean Protein Concentrates” in “Technology of production of edible flours and protein products from soybeans” by Berk FAO Agricultural Services Bulletin No. 97 (1992). The resulting protein fraction will be enriched in proteins, but also still contain other components. Protein may optionally be further purified from this fraction using any suitable method.

In one embodiment, the process of the aspects of the disclosed embodiments relate comprises the further step of hydrolysing the protein fraction obtained from the process of the aspects of the disclosed embodiments relate to obtain amino acids and small peptides.

In one embodiment of the process of the aspects of the disclosed embodiments, the process comprises the further step of producing a food or feed product from said biomass, from said protein fraction or from said fraction comprising non-protein components. Said further step may simply comprise incorporating said biomass, protein fraction or fraction comprising non-protein components in a food or feed product, by adding it during the production of the food or feed product. In other embodiments, further purification or modification of the biomass or fraction thereof is performed during the course of its incorporation into a food or feed product.

In a further aspect, the aspects of the disclosed embodiments relate to a product, such as biomass, protein, or non-protein components, obtained or obtainable by the process according to the aspects of the disclosed embodiments relate .

In one embodiment, the product obtained from the process of the aspects of the disclosed embodiments comprises more than 40% protein, such as between 40% and 99% protein, e.g. between 40% and 90% protein, such as between 40% and 60% protein. In a particular embodiment, the product comprises between 25% and 75% protein, between 0% and 20% lipid and between 5% and 40% carbohydrates. In a further embodiment, the product comprises between 40% and 60% protein, between 0% and 15% lipid and between 10% and 25% carbohydrate. In an even further embodiment, the product obtained from the process of the aspects of the disclosed embodiments comprises between 45% and 55% protein, between 5% and 10% lipid and between 10% and 20% carbohydrates.

As described above, the aspects of the disclosed embodiments in a further aspect relate to a food or feed product obtained or obtainable by the process according to the aspects of the disclosed embodiments. When used herein, the terms “food” and “feed” are intended to include not only conventional food and feed products, such as processed foods, but also related products, such as food and feed supplements, e.g. protein bars, powders or shakes, meat replacements, food ingredients, probiotics, prebiotics, nutraceuticals and the like. In certain embodiments, said biomass, said protein fraction or said fraction comprising non-protein components is utilized in the production of a vegetarian or vegan food product.

In further aspects, the aspects of the disclosed embodiments relate to the production of pharmaceuticals, bioactive compounds, nutraceuticals, antioxidants and/or vitamins using bacteria of strain VTT-E-193585 or variants thereof, such as the variants described herein, for example strain VIT E-213595. Bioactive compounds, nutraceuticals, antioxidants, vitamins may be extracted from the biomass using methods known in the art.

Thus, in further embodiments, the aspects of the disclosed embodiments relate to the methods for production of biomass described herein comprising a further step of isolating, such as extracting, compounds, such as bioactive compounds, nutraceuticals, antioxidants, vitamins, from said biomass or from the cultivation liquid. In further embodiment, the aspects of the disclosed embodiments relate to the use of these compounds in a pharmaceutical product or as a food supplement. In one embodiment, the extracted compound is beta-carotene (provitamine A). In another embodiment, the extracted compound is ubiquinone Q10. In another embodiment, the extracted compound is a form of heme iron e.g. cytochrome C. In another embodiment, the extracted compound is vitamin B12.

The aspects of the disclosed embodiments is further illustrated with the following, non-limiting, examples:

### EXAMPLES

### Example 1. Isolation of Bacterial Strain Capable of Chemoautotrophic Growth

A sample of 50 mL containing soil and seawater was collected in a sterile falcon tube from the seashore of the Baltic sea in Naantali in Finland. Part of soil sample was mixed with 10 ml of mineral medium in a sterile Erlenmeyer flask. The medium consisted of 1 g/L NH₄OH, 0.23 g/L KH₂PO₄, 0.29 g/L Na₂HPO₄·2 H₂O, 0.005 g/L NaVO₃·l H₂O, 0.2 g/L FeSO₄·7 H₂O, 0.5 g/L MgSO₄·7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L CoSO₄, 0.00005 g/L CuSO₄ and 0.0001 g/L NiSO₄ prepared in tap water. The suspension of soil and medium was incubated in a shaking incubator in +30° C. temperature in a sealed steel box that was flushed continuously with a gas mixture: 150 ml/min of N₂, 18 ml/min of H₂, 3 mL/min of O₂ and 6 ml/min of CO₂. The cultivation was refreshed in seven-day intervals by taking 1 mL of suspension, which was added in sterile conditions to 9 mL of medium in Erlenmeyer flask, and then placed back into the incubation box. After the fourth dilution, there was no noticeable soil left in the suspension. The volume of the cell suspension was increased to 100 mL in order to grow biomass for bioreactor cultivation. The optical density (OD₆₀₀) of the suspension was 1.53 when it was inoculated to 190 mL of mineral medium in 15-vessel 200-mL parallel bioreactor system (Medicel Explorer, Medicel Oy, Finland). The cultivation conditions were 800 rpm agitation, +30° C. temperature and the pH was set to 6.8, controlling it with 1 M NaOH. Gas was fed through a sparger with a gas mixture consisting of 14 mL/min H₂, 3 mL/min O₂ and 6 mL/min CO₂. The head space of the reactor was flushed with 300 ml/min air. Continuous cultivation was fed with mineral medium 6 mL/h and cell suspension was drawn from the reactor via capillary keeping the volume constant at 200 mL. Cell suspension drawn from the reactor was stored at +4° C. A sample was taken from the bioreactor automatically every day, and absorbance at 600 nm was measured to monitor the growth. After 498 hours of bioreactor cultivation, samples were drawn aseptically and suspension was diluted and plated to agar mineral medium plates containing the above minerals and 2% bacteriological agar. Plates were incubated in same conditions as described above for the Erlenmeyer flasks. Colonies were then picked from agar plates and streaked to new agar plates in order to isolate one organism in one colony. This was repeated twice. Single colonies were picked and suspended into 200 μL of medium in a 96-well microtiter plate. The suspension was incubated at +30° C. temperature and shaken 625 rpm in an EnzyScreen gas tight box that was flushed continuously with 150 ml/min of N₂, 18 mL/min of H₂, 3 mL/min of O₂ and 6 ml/min of CO₂. The suspension from one well was transferred to an Erlenmeyer flask and supplemented with fresh medium. Volume was increased until there was enough biomass to perform a bioreactor cultivation. The organism was deposited in the VTT culture collection as VTT-E-193585.

16S rRNA sequencing of a sample demonstrated that the sample contained only one organism. The same sample was used for Illumina NextSeq sequencing providing 1×150 bp metagenomic shotgun sequences. Using Unicycler (Wick et al, 2017 PLOS computational biology 13:e1005595), the de novo assembly was made for metagenomic sequences consisting of 101 contigs. The total genome length was 4,846,739 bp and the GC content was 67.9%. Gene predictions and functional annotations were performed using Prokka (Seemann, 2014 Bioinformatics 30:2068). The genome annotation produced 4,429 genes. Roary pan genomic alignment (Page et al, 2015 Bioinformatics 31:3691) grouped VTT-E-193585 among Xanthobacter species. The strain was therefore identified as a Xanthobacter sp., the closest genome being Xanthobacter tagetidis. Alignment-based calculation of average nucleotide identity that takes into account only orthologous fragments (OrthoANI) (Lee et al, 2016 Int J Syst Evol Microbiol 66:1100) gave the best match of 80.4% to Xanthobacter tagetidis (ATCC 700314; GCF_003667445.1), whereas the proposed species boundary cut-off is 95-96% (see e.g., Chun et al., 2018 Int J Syst Evol Microbiol, 68: 461-466). Xanthobacter autotrophicus Py2 gave a match of 79.6%, while the match for Xanthobacter sp. 91 was 79.0%. It could thus be concluded that the isolated bacterial strain deposited as VTT-E-193585 belongs to the Phylum: Proteobacteria; to the Class: Alpha Proteobacteria; and to the Order: Rhizobiales. The most probable Family is Xanthobacteraceae, and the Genus Xanthobacter. The VTT-E-193585 bacterial strain could not be assigned unequivocally to any known species.

A search for putative antimicrobial resistance genes was performed. The ABRicate (https://github.com/tseemann/abricate) tool was used to search the genome against the Arg-Annot, NCBI, ResFinder, the ecOH, Megares and VFDB databases using blastn or blastp. A threshold of 50% was set for both identity and coverage, both on nucleotide and protein level. Only two putative antimicrobial resistance genes were identified. These two genes did not contain amino-acid changes linked to antibiotics resistance and thus a resistant phenotype is not expected.

### Example 2. Pilot Cultivation and Analysis of Isolated Bacterial Strain

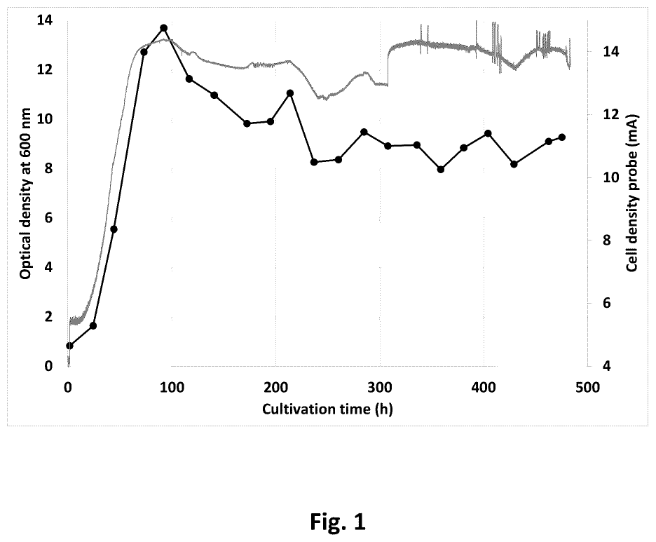

The isolated bacterial strain deposited as VTT-E-193585 was cultivated in a conventional 200-liter stirred tank bioreactor (MPF-U, Marubishi Ltd, Japan). Mixing was performed with Rushton-type impellers rotating at 400 rpm. Temperature in the cultivation was maintained at +30° C. pH was maintained at 6.8±0.2 by adding 8 M NaOH or 3.6 M H₃PO₄ by software control. Cultivation medium contained 1 g/L NH₄OH, 0.23 g/L KH₂PO₄, 0.29 g/L Na₂HPO₄·2 H₂O, 0.005 g/L NaVO₃·H₂O, 0.2 g/L FeSO₄·7 H₂O, 0.5 g/L MgSO₄·7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L CoSO₄, 0.00005 g/L CuSO₄ and 0.0001 g/L NiSO₄ prepared in tap water. A mixture containing 1.8-10.5 L/min hydrogen gas, 0.6-2.5 L/min oxygen gas and 1.8-5 L/min carbon dioxide gas was supplied constantly as the main source of energy and carbon. Dissolved oxygen level was maintained at 7.2±0.5% by adjusting the gas mixture composition. The inoculum for the cultivation was prepared as described in Example 1. Growth was monitored by taking samples manually and analysing the cell density as optical density by measuring absorbance at 600 nm (Ultrospec 2100 pro UV/visible spectrophotometer, Biochrom Ltd., England) and by measuring cell dry weight (CDW) by drying in oven overnight at 105° C. Optical density was also monitored by using an in situ absorbance probe (Trucell 2, Finesse Ltd, USA). A growth curve of the cultivation is presented in FIG. 1. The maximum growth rate in batch phase was 0.06 h⁻¹. The maximum cell density was 4.5 g_CDW/L at 92 h. After 92 h of cultivation, feed of fresh cultivation medium as described above was started at a dilution rate of 0.01 h⁻¹. During the continued feed, the cell density was on average 2.9 g_CDW/L. Cultivation liquid was constantly collected to a cooled (+10° C.) tank from which it was fed in 300-liter batches to a continuous centrifugal separator (BTPX-205, Alfa-Laval AB, Sweden). The cell-containing slurry collected from the separator was fed into an atmospheric double drum dryer (Buflovak 6×8 ADDD, Hebeler process solutions Llc., USA), heated with 4 bar steam and drums rotating at 3.5 rpm. This resulted in dried cell powder with approximately 96% dry matter content. Analysis results of the dried cell powder are presented in Table 1 for the proximate composition, in Table 2 for the amino acid composition, in Table 3 for the fatty acid composition, and in Table 4 for the vitamin content. Analyses demonstrate that the dried cell powder has high protein content with all the essential amino acids. It also contains more unsaturated than saturated fatty acids and a lot of B-group vitamins. Peptidoglycan content was only 0.002 mg/g_CDW and lipopolysaccharide content was 0.01 mg/g_CDW. It would be beneficial that these concentrations would be as small as possible. In comparison, in a commercial lactic acid bacteria preparation analysed at the same time, the peptidoglycan content was 0.244 mg/g_DW and the lipopolysaccharide content was 0.015 mg/g_DW. Cytotoxicity and genotoxicity assays were performed using the supernatant samples of cultivation. No cytotoxicity against HepG2 or HeLa229 human cell lines was observed. No genotoxicity against Escherichia coli WP2 trp- or CM871 uvrA recA lexA strains was observed.

TABLE 1
Analysis results of dried cell powder of isolated bacterial strain
deposited as VTT-E-193585.

| Parameter | Method | Unit | | Value |
|---|---|---|---|---|
| Moisture | Drying at 103° C. | g/100 | g | 3.3 |
| Protein | Kjeldahl (N × 6.25) | g/100 | g | 72.2 |
| Fat | Weibull-Stoldt | g/100 | g | 6.0 |
| Saturated fatty acids | Calculation based | g/100 | g | 1.8 |
| | on Table 3 | | | |
| Mono-unsaturated | Calculation based | g/100 | g | 3.8 |
| fatty acids | on Table 3 | | | |
| Polyunsaturated | Calculation based | g/100 | g | 0.4 |
| fatty acids | on Table 3 | | | |
| Omega 3 fatty acids | Calculation based | g/100 | g | <0.01 |
| | on Table 3 | | | |
| Omega 6 fatty acids | Calculation based | g/100 | g | 0.4 |
| | on Table 3 | | | |
| Dietary fibres | Gravimetric | g/100 | g | 10.5 |
| Ash | Ashing at 550° C. | g/100 | g | 5.8 |
| Glucose | HPLC-ELSD | g/100 | g | <0.15 |
| Fructose | HPLC-ELSD | g/100 | g | <0.1 |
| Sucrose | HPLC-ELSD | g/100 | g | <0.1 |
| Lactose | HPLC-ELSD | g/100 | g | <0.25 |
| Maltose | HPLC-ELSD | g/100 | g | <0.2 |
| Total sugars | Calculation | g/100 | g | <0.8 |
| Carbohydrates | Calculation | g/100 | g | 2.2 |
| Energy | Calculation | kJ/100 | g | 1572 |
| Energy | Calculation | kcal/100 | g | 373 |

TABLE 2
Amino acid composition of dried cell powder of isolated bacterial
strain deposited as VTT-E-193585.

| Parameter | Method | Unit | Value |
|---|---|---|---|
| Lysine | Ion chromatography | % | 3.95 |
| Methionine | Ion chromatography | % | 1.60 |
| Cystine | Ion chromatography | % | 0.39 |
| Aspartic | Ion chromatography | % | 6.82 |
| Threonine | Ion chromatography | % | 3.47 |
| Serine | Ion chromatography | % | 2.75 |
| Glutamic | Ion chromatography | % | 8.84 |
| Proline | Ion chromatography | % | 3.14 |
| Glycine | Ion chromatography | % | 4.40 |
| Alanine | Ion chromatography | % | 6.94 |
| Valine | Ion chromatography | % | 4.96 |
| Isoleucine | Ion chromatography | % | 3.34 |
| Leucine | Ion chromatography | % | 6.08 |
| Tyrosine | Ion chromatography | % | 2.99 |
| Phenylalanine | Ion chromatography | % | 4.58 |
| Histidine | Ion chromatography | % | 1.66 |
| Arginine | Ion chromatography | % | 4.96 |
| Tryptophan | HPLC | % | 1.34 |

TABLE 3
Fatty acid composition of dried cell powder of isolated bacterial
strain deposited as VTT-E-193585.

| Parameter | Method | Unit | Value |
|---|---|---|---|
| C16:0 (Palmitic acid) | GC-MS | % | 24.8 |
| C16:1 (Palmitoleic acid) | GC-MS | % | 3.0 |
| C18:0 (Stearic acid) | GC-MS | % | 4.4 |
| C18:1n9 (Oleic acid) | GC-MS | % | 59.9 |
| C18:2n6 (Linoleic acid) | GC-MS | % | 6.1 |
| C18:3n3 (alpha-Linolenic acid) | GC-MS | % | 0.4 |

TABLE 4
Vitamin content of dried cell powder of isolated bacterial
strain deposited as VTT-E-193585.

| Parameter | Method | Unit | | Value |
|---|---|---|---|---|
| Vitamin A (RE) | HPLC | IU/100 | g | <100 |
| Vitamin E (TE) | HPLC | mg/100 | g | 0.33 |
| Vitamin D3 | HPLC | IU/100 | g | <10 |
| Vitamin D2 | HPLC | IU/100 | g | 21.6 |
| Vitamin C | HPLC | mg/100 | g | <1 |
| Thiamine chloride | LC-MS/MS | mg/100 | g | 0.9 |
| Hydrochloride | | | | |
| Vitamin B1 (Thiamine) | Calculation | mg/100 | g | 0.708 |
| Vitamin B2 (Riboflavin) | HPLC | mg/100 | g | 6.27 |
| Pyridoxine hydrochloride | HPLC | mg/100 | g | 3.39 |
| Vitamin B6 (Pyridoxine) | Calculation | mg/100 | g | 2.79 |
| Vitamin B12 | LC-MS/MS | mg/100 | g | 224 |
| Choline chloride | LC-MS/MS | mg/100 | g | 14.3 |
| Biotin | LC-MS/MS | mg/100 | g | 15.6 |
| Folic acid | Microbiological | mg/100 | g | 1270 |
| Niacin (Vitamin B3) | Microbiological | mg/100 | g | 23.2 |
| Pantothenic acid | Microbiological | mg/100 | g | 6.53 |

### Example 3. Cultivation of Isolated Bacterial Strain on Different Nitrogen Sources

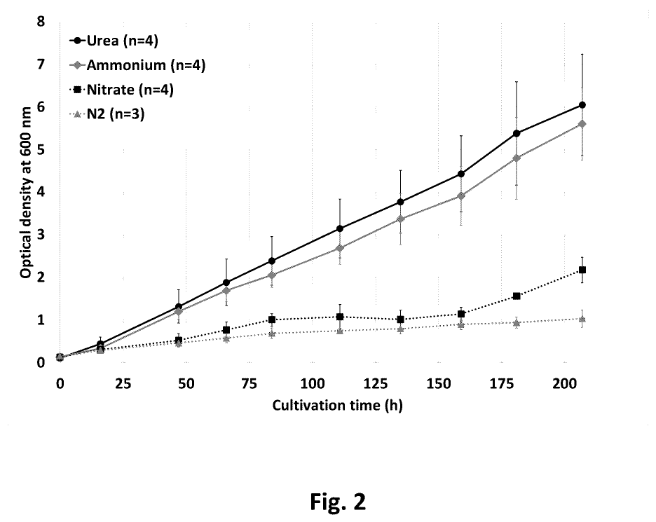

The isolated bacterial strain deposited as VTT-E-193585 was cultivated in a 15-vessel parallel bioreactor system at 200 mL volume (Medicel Explorer, Medicel Oy, Finland). Mixing was performed with Rushton-type impellers rotating at 800 rpm. The temperature in the cultivation was maintained at +30° C. pH was maintained at 6.8 by adding 1 M NaOH. The cultivation medium contained 0.23 g/L KH₂PO₄, 0.29 g/L Na₂HPO₄·2 H₂O, 0.005 g/L NaVO₃·H₂O, 0.2 g/L FeSO₄·7 H₂O, 0.5 g/L MgSO₄·7 H₂O, 0.01 g/L CaSO₄, 0.00015 g/L Na₂MoO₄·2 H₂O, 0.005 g/L MnSO₄, 0.0005 g/L ZnSO₄·7 H₂O, 0.0015 g/L H₃BO₃, 0.001 g/L CoSO₄, 0.00005 g/L CuSO₄ and 0.0001 g/L NiSO₄ prepared in tap water. Furthermore, the nitrogen source was varied in the cultivations so that four cultivations contained 18.7 mM NH₄OH, four cultivations contained 9.34 mM urea (OC(NH₂)₂), four cultivations contained 18.7 mM potassium nitrate (KNO₃), and three cultivations were left without nitrogen source in the medium. A mixture containing 22 mL/min hydrogen gas, 3.2 mL/min air and 6.4 mL/min carbon dioxide gas was supplied constantly as the main source of energy and carbon. Thus, with air, all cultivations were also supplied with nitrogen gas. Growth was monitored by taking samples automatically and analysing the cell density as optical density by measuring absorbance at 600 nm (Ultrospec 2100 pro UV/visible spectrophotometer, Biochrom Ltd., England). Growth curves of the cultivations are presented in FIG. 2. Growth on ammonia and urea were comparable. Growth on nitrate or nitrogen gas was clearly slower than on ammonia or urea. Towards the end of the cultivation, the growth on nitrate was better than growth on nitrogen gas as the only source of nitrogen. There was nonetheless growth also in the cultivations in which nitrogen gas was the only source of nitrogen demonstrating that isolated bacterial strain deposited as VTT-E-193585 is capable of nitrogen fixation.

### Example 4. Characterization of Antibiotic Susceptibility

Antibiotic susceptibility of gentamicin, kanamycin, streptomycin, tetracycline, ampicillin, ciprofloxacin, colistin and fosfomycin for the isolated bacterial strain deposited as VTT-E-193585 was analysed according to CLSI M07-A111 standard (Clinical and laboratory standards institute. Methods for dilution antimicrobial susceptibility tests for bacteria that grow aerobically, 11th ed. CLSI standard M07, 2018) with hand-made microdilution plate for ampicillin, ciprofloxacin and colistin, with VetMIC Lact-1 plate (SVA National Veterinary Institute, Uppsala, Sweden) for gentamicin, kanamycin, streptomycin and tetracycline using broth microdilution method and for fosfomycin using agar dilution method in aerobic conditions at +35±2° C. for 48±1 hours using cation-adjusted Mueller Hinton Broth -medium (LabM, LAB114, cations Mg²⁺ and Ca²⁺ added separately). Escherichia coli ATCC 25922 was used as quality control strain and it was incubated in aerobic conditions, at +35±2° C. for 18±2 hours. Results of antibiotic susceptibility of strains are presented in Table 5. The isolation bacterial strain was found to be generally sensitive to antibiotics. For gentamicin, kanamycin, streptomycin and tetracycline minimum inhibitory concentration (MIC) values for VTT-E-193585 were lower or comparable to E. coli ATCC 25922, while for ampicillin, ciprofloxacin, colistin and fosfomycin the MIC values were higher in VTT-E-193585.

TABLE 5
Minimum Inhibitory Concentration (MIC, μg/ml) -values of antibiotics
for VTT-E-193585 strain and Escherichia coli ATCC 25922

| | VTT-E-193585 | E. coli ATCC 25922 |
|---|---|---|
| | 48 h ± 1 h | 18 h ± 2 h |
| Gentamicin | 0.5 | 0.5 |
| Kanamycin | 2 | 4 |
| Streptomycin | 0.5 | 4 |
| Tetracycline | 0.25 | 1 |
| Ampicillin | 16 | 8 |
| Ciprofloxacin | 0.06 | 0.008 |
| Colistin | 4 | 2 |
| Fosfomycin | 32 | 0.5 |

### Example 5. Construction of phaC Knockout Strains

Media compositions per 1 L of liquid.

### DSM81-LO4 (DSM)

| | | |
|---|---|---|
| KH₂PO₄ | 2.3 | g |
| Na₂HPO₄•2H₂O | 2.9 | g |
| Na₂SO₄ | 5.45 | g |
| (NH₄)₂SO₄* | 1.19 | g |
| MgSO₄•7H₂O | 0.5 | g |
| CaSO₄•2H₂O | 11.7 | mg |
| MnSO₄•H₂O | 4.4 | mg |
| NaVO₃ | 5 | mg |
| NaHCO₃ | 0.5 | g |
| Ammonium iron (III) citrate | 5 | mg |
| ZnSO₄•7H₂O | 0.5 | mg |
| H₃BO₃ | 1.5 | mg |
| CoCl₂•6H₂O | 1 | mg |
| CuCl₂•2H₂O | 50 | μg |
| NiCl₂•6H₂O | 0.1 | mg |
| Na₂MoO₄•2H₂O | 0.15 | mg |
| Riboflavin | 0.5 | mg |
| Thiamine-HCl•2 H₂O | 2.5 | mg |
| Nicotinic acid | 2.5 | mg |
| Pyridoxine-HCl | 2.5 | mg |
| Ca-pantothenate | 2.5 | mg |
| Biotin | 5 | μg |
| Folic acid | 10 | μg |
| Vitamin B₁₂ | 50 | μg |

*altered for GC-MS analysis

### Lysogeny Broth (LB)

| | |
|---|---|
| Yeast Extract | 5 g |
| Tryptone | 10 g |
| NaCl | 10 g |
| Agar (for plates) | 15 g |

### Super Optimal Broth (SOB)

| | |
|---|---|
| Tryptone | 20 g |
| Yeast extract | 5 g |
| NaCl | 0.58 g |
| KCl | 0.18 g |
| MgCl₂ | 0.95 g |
| MgSO₄ | 1.20 g |

### Super Optimal Broth With Ethanol (SOBE)

5.8 mL of ethanol/1 L of SOB

### Tryptic Soy Agar (TSA)

BBL™ Trypticase™ Soy Agar (BD) 40 g

### Cultivation Methods

All SoF1 cultures in heterotrophic growth conditions were cultivated with 220 rpm shaking at 30° C. in 10 mL volume of super optimal broth supplemented with 100 mM of ethanol (SOBE). All SoF1 cultures in autotrophic conditions were cultivated in DSM81-LO4 media (DSM) with 136 rpm shaking and at the same temperature and volume as in heterotrophic growth conditions. The gas composition in autotrophic conditions was the following: 44% CO₂, 26% N₂, 22% H₂, 7% O₂, and 1% other gases.

Escherichia coli Strains

Used E. coli strains and their relevant characteristic are summarized in Table 6. All strains were grown on lysogeny broth (LB). Antibiotics were used in the following concentrations: 100 μg/mL of ampicillin (AMP), 50 μg/mL of kanamycin (KAN) and 10 μg/ml of tetracycline (TET).

TABLE 6
E. coli strains used

| E. coli strain | Relevant characteristics |
|---|---|
| TOP10 | Electrocompetent used for plasmid |
| | construction |
| S17-1 (Simon et al. 1983 | Chemically competent, chromosomally |
| Nat Biotechnol 1: 784-791) | integrated RP4 derivative |
| JM109(DE3) | Nalidixic acid resistance |

### Plasmid Construction

The sequencing of the bacterial genome of strain VTT-E-193585 described in Example 1 identified genes phaC1 (SEQ ID NO:60, encoding the protein set forth in SEQ ID NO:62) and phaC2 (SEQ ID NO:61, encoding the protein set forth in SEQ ID NO:63) with homology to phaC genes found from other Xanthobacter spp., encoding for polyhydroxyalkanoate (PHA) synthases.

Two plasmids were constructed to target deletion of phaC1 and phaC2 genes in the genome of SoF1 (Table 7). The flanking 1000 bps left (LHA) and right (RHA) homology arms of phaC1 and phaC2 were amplified from the genomic DNA of SoF1 with oligos (8 oligos). Both plasmids were constructed from pUC57 with Gibson assembly. Kanamycin resistance gene (kan), tetracycline resistance gene (tet) and the mobilization region (mob) sequences were the same as used in plasmids described in Van den Bergh et al. 1993 J Bacteriol 175:6097-6104.

TABLE 7
Summary of plasmids constructed

| Plasmid | Backbone | Deletion cassette | phaC target |
|---|---|---|---|
| pSF001 | pUC57m (amp, mob) | LHA-kan-RHA | phaC1 |
| pSF002 | pUC57m (amp, mob) | LHA-tet-RHA | phaC2 |

### Knockout Strain Construction

Plasmids were transferred to SoF1 with conjugation or electroporation. Antibiotic concentrations used for selection of modified SoF1 strains were 20 μg/mL of KAN and 10 μg/mL of TET.

For conjugation, liquid culture (LC) of SoF1 was grown in autotrophic conditions as described above for two to three days to reach an OD of 0.7-1. Overnight (O/N) LC of E. coli S17-1, with and without plasmid, and JM109(DE3) were grown at 37° C. with shaking 220 rpm. New LCs were inoculated from O/N cultures next day and grown to exponential phase (OD 0.3-0.6). E. coli cells were centrifuged 5900 rpm 30 s, washed, and resuspended to 1 volume of 0.9% NaCl. E. coli and SoF1 cells were mixed with OD ratio 1:15 and DSM media was added up to 1 mL. S17-1 without plasmid mixed with SoF1 was used as a negative control and JM109(DE3) with S17-1 containing plasmid was used as a positive control. Mixtures were vacuum filtered through a 0.22 um GV Durapore® membrane filter (MilliporeSigma, US). Filters were placed on prewarmed TSA plates, cells facing away from the agar, and incubated O/N in autotrophic conditions. Next day, filters were washed with 1 ml of 0.9% NaCl, vortexed and centrifuged 4000 rpm for 1 min and, after removing the filters, cells were resuspended. Cells were plated with serial dilutions to selective plates and grown in appropriate conditions. Conjugated JM109(DE3) control was plated on TSA plates containing 40 μg/mL of nalidixic acid for selection and incubated at 37° C. O/N. Conjugated SoF1 cultures were plated on DSM plates containing KAN and incubated in autotrophic conditions approximately for one week. After colonies appeared on SoF1 plates, they were reapplied to fresh DSM KAN plates and incubated one more week in autotrophic conditions. Single colonies from these plates were tapped on TSA plates and grown in 30° C. in heterotrophic conditions. If growth on TSA plates resulted in E. coli growth, colonies were reapplied again to selective DSM plates.

For electroporation, LC of SoF1 was cultivated in autotrophic conditions for two to three days to reach an OD of 0.7-1.5. Cells were transformed to Falcon tubes and chilled on ice for 15-30 min. Cells were centrifuged at 4° C. 4000 rpm for 5-10 min, supernatant was discarded, and the pellet was resuspended to 1 volume of ice-cold double distilled H₂O. Centrifugation was repeated and supernatant was discarded. Washing was repeated with 1 volume of ice-cold 10% glycerol. Cells were resuspended to ice-cold 10% glycerol to reach concentration of around 2·10¹⁰ cells/mL. Cells were used immediately for electrotransformation. 40 μL of cells were mixed with 1 μL of plasmid and incubated for 10 min on ice. Cells were transformed to an electroporation cuvette on ice. The cuvette was subjected to a single electric pulse of 2.5 kV with 25 μF capacitance and 400 or 600Ω resistance. 1 mL of pre-warmed (30° C.) SOBE was added immediately and solution was transferred to a Falcon tube and incubated O/N at autotrophic conditions. After incubation, cells were plated on TSA selection plates with multiple dilutions and incubated in heterotrophic conditions.

After transformation with conjugation or electroporation, successful transformants were screened with colony PCR with addition of final concentration of 3% dimethyl sulfoxide to the standard PCR mixture. Constructed SoF1 strains are summarized in Table 8.

TABLE 8
Constructed SoF1 variant strains.

| Strain | Genotype |
|---|---|
| SoF1-2.0 | phaC1::kan |
| SoF1-3.0 | phaC2::tet |
| SoF1-4.0 | phaC1::kan and phaC2::tet |

### PHB Content Analysis

PHB contents of the wild type (WT) strain SoF1 and the knockout strain SoF1-2.0 were analysed with gas chromatography-mass spectrometry (GC-MS). Both strains were grown in autotrophic conditions as described above in 5 mL cultivation volume in varying nitrogen concentrations (18.0, 13.5, 9.0 and 4.5 mM of nitrogen) and in heterotrophic conditions. Cultivations were inoculated from cultures grown in autotrophic conditions to gain a starting OD of 0.1. Analysis was done at earliest after one week from inoculation. 1-3 mL of each sample was centrifuged down, and the pellet was stored at −20° C. Pellets were thawed, washed twice with double distilled H₂O and lyophilized for 24-48 h. 10 mg of each sample was subjected to methanolysis by heating at 100° C. for 140 min in a solution containing 1 mL chloroform, 150 μL sulfuric acid, 20 μL internal standard (3-hydroxybutyric acid), and 830 μL methanol. 3-hydroxybutyric acid was treated similarly as a reference sample. After samples were cooled to room temperature, water-soluble particles were removed with 0.5 ml of water. Gas chromatography system (7890, Agilent) and HP-FFAP column (19091F-102, Agilent) were used to analyse the chloroform phase.

### Results

According to GC-MS, almost no PHB was produced in the SoF1-2.0 strain whereas in the WT SoF1 the PHB dry content was 15-30% when grown in autotrophic conditions (Table 9). Almost no PHB was produced in SoF1 or SoF1-2.0 when grown in heterotrophic conditions

TABLE 9
PHB contents of WT SoF1 and SoF1-2.0 grown in autotrophic
conditions in different nitrogen concentrations in autotrophic conditions
and in heterotrophic conditions (SOBE) determined via GC-MS.

| Nitrogen | SoF1 | SoF1-2.0 |
|---|---|---|
| concentration | (mg of PHB/g of dry | (mg of PHB/g of |
| (mM) | cell mass) | dry cell mass) |
| 4.5 | 312.9 | 0.3 |
| 9.0 | 230.0 | 1.3 |
| 13.5 | 251.9 | 1.3 |
| 18.0 | 204.0 | 1.1 |
| Not known, grown in | 0.1 | 0.2 |
| SOBE | | |

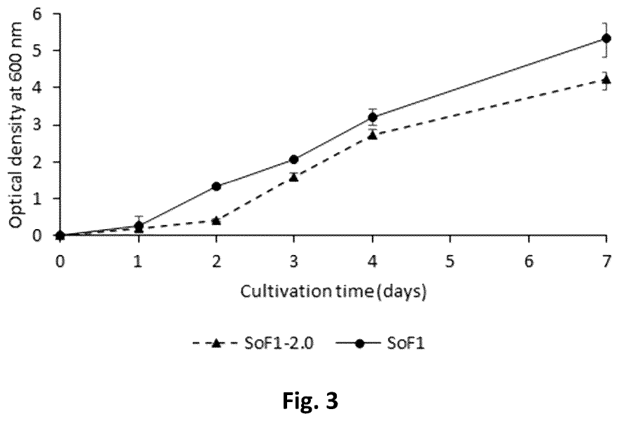

Autotrophic growth curves of SoF1 and SoF1-2.0 are presented in FIG. 3. SoF1-2.0 has a slightly lower growth rate compared to SoF1.

phaC1 DNA sequence (SEQ ID NO: 60):
ATGTCCGCAGCCGAGGAAACGTCCACGCACGCTGAATTGAGGCTCCCGCAGGATGGGGGG
AGCATGACGTCGCGGCCGCCGAGGCCGCGGTGGACCGGTCCGCCGGGGAGCCGTCGGGAA
CGCAGGCGTCCGCCGCGCCCGCCGAGGCGGCGCCGTCTTCCGCTCCTGTTTCCGCTGCTGCC
GGAGAAACGCAGCCACAGGACGATACCCCGCCGCAGGACCCGTCTTCCCTGTCGGACGGTCC
AGGCCTGTCGCCGCCCGTGACCCAGCCCGGCGCGGCGTCCGGCGACTTCGGCGGGCCCGAG
GCCATGGGCGACGCCTTCATGCCGCCTGTGCCGGAAGAGCCCATGGAGGGGATGGCCCCCG
CGCCGGCAATCTCCGCGGCGCCCGCCTCGGTGCCGTCTGCCGGCTGGCCGGAGGATGCGCC
TTCGGCACTGCTCGATGCGGTGGATGCGTCCGGGGACCTGCCGGCTGCCGCGGAGGGCGCG
GCCACGGCGCCGGAGCCCATTTTCCGCGAGCTGCCCATGACGGCGGCGCCCGCCGCACCTG
CCATCGCGAACATTCTCGAGCCGGTGGCCGAGGCTCTGTCCGCGCTCGGGGCGGTGGCCGT
CTCCCGGCCCCAGGTCCAGCGCGAATTCCGCGCGGCGCCCGAACACCCCCCCATGCCCAGG
ATGGCGCCCCCTCAGGCGGCGCCAGAACCGGCCCCTGCCGTTCCGCCCAAACCTGAAGCTGC
CAAACCTGAAGCTGCCAAACCTGAAGCCGCCAAGCCTGAAGCTGCCAAGCCTGAAGTTGCCA
AGCCGGACGCCGCCGCGCCGGACGCCGCGAAATCTTCGGGCAAGGCGGAGCGTCCGTCCGG
CGCCGGGGACGGCTCTGGAACGTCGGGCGTGAACATGGAGGCCTTCTCCCGCAACCTCGCC
CGTCTGGTGGAAGAGGGCGGCAAGGCCATGGCCGCCTATCTGAGCCCACGGGAGCAGGGCA
AGACCGACGATCTCGCCGATGACATCGCCGATGCCATGAAGACCGTGGGCCAGGTGGTGGA
ATACTGGGTCGCCGATCCCCAGCGCACGGTGGAGGCGCAGTCCCGCCTCATGGGCGGCTAT
CTCTCGGTCTGGGCCAACACCCTGAAGCGCCTGGCAGGCGAGGAGGCCACGCCGGTGGCCG
CCCCCGACCCGAAGGATGCCCGCTTCAAGGATGCCGGCTGGAACGACAGCCCCATGTTCGAT
GCCCTCAAGCAGGCCTATCTGGTCACCTCGGACTGGGCGCAGAACATGGTGGACGAGGCCAA
GGGCCTCGATCCCCACACCAAGCACAAGGCGGAGTTCCTGGTGCGGCAGATCGCCAACGCCA
TCTCGCCCTCCAACTTCGTCCTCACCAATCCCGAGCTCATCCGCGAGACCCTGCACTCGTCGG
GCGAGAATCTGGTGAAGGGCATGCAGAACCTCACCGCGGATCTGATGGCGGGGCAGGGCAC
GCTGAAGATCCGCCAGACGGATCTTTCCGCCTTCGAGGTGGGCCGCAACCTCGCCACCACGC
CGGGCAAGGTGATCTTCGAAAACGAGCTGATGCAGCTCATCCAGTACGAGCCGACCACCGAG
ACGGTGAAGAAGACGCCGGTGCTCATCGTCCCGCCCTGGATCAACAAGTTCTACATCCTCGA
CCTGACGGCGGAAAAATCCTTGATCAAGTGGCTGGTGAGCCAGGGACTGACGGTCTTCACCA
TCTCCTGGGTCAATCCCGACGGGCGCCTCGCCGCCAAGGGCTTCGACGATTACATGCGCGAC
GGCATCATGGCCGCCCTCGACGCGGTGGCGGTGGCCTCCGGCGAGCGGCGGGCCCATGCG
GTGGGCTATTGCGTGGGCGGCACGCTGCTGGCCACCACGCTCGCTTACATGGCCGCCACCG
GGGATGACCGCATCGCCAGCGCCACCTTCCTCACCACCCAGATCGACTTCACCCATGCGGGC
GATCTGAAGGTGTTCGTGGACGAAAGCCAGCTCGCCACCATCGAGCGCAAGATGAAGGAGAT
GGGGTATCTGGAAGGCTCGAAGATGGCCTCCGCCTTCAACATGCTGCGCTCCAACGACCTGA
TCTGGCCCTATGTGGTGAACAACTACATGAAGGGCAAGGCGCCGTTTCCGTTCGACCTGCTGT
TCTGGAATTCGGATTCCACCCGCATGCCGGCGGCGAACCATTCCTATTATCTGCGCAACTGCT
ACCTCACCAACAATATCGCCCGGGGTCTGGCGGAGCTGGCCGGCCTCAAGATCGATGTCACC
AAGGTGTCGATCCCAGTCTATTCGCTGGCGACGCGGGAAGACCACATCGCCCCGGCCAACTC
GGTCTATATCGGGGCAAATCTCCTGTCCGGCCCGGTGCGCTACGTGCTGGCGGGCTCGGGCC
ACATCGCCGGGGTGGTGAACCCGCCGGCCAAGATGAAGTATCAGTACTGGGCCGACGGCCC
GGTGGGGCCGAGCTACGAGGCCTGGCTGGCCGGGGCGCAGGAGCACAAGGGCTCCTGGTG
GCCGGACTGGTTCAACTGGTTCTCCTTCAACCATCCCGAGGAGGTGCCGGCGCGCGCCATCG
GCGGCGGCCGCCTCGCCCCCATCGAGGACGCACCCGGGCGCTATGTGAAGGAGCGGTCGTA
G
phaC2 DNA sequence
(SEQ ID NO: 61)
ATGGAAGCGCGAAAGATGCCCGTTTCACCCCCCTCCTCCGCCACCATCCTGCCCCTGCCCGTC
TCGGCCGCTCCCCCTTCGACGGCGCCCGCCGCGTCCCTGCCGGCGACGGCCTCCAGCTCCAC
CAACAAGGCCAGCCCCTCAGCCTTCCCCGCCGCCTGGGCGCGTTCCTTCGCCCTGCCCGGCC
TGCCCGCCTTCCTGTGCCCGGACGAGGAGGATTTCGAGGAGGGTTCGGGGCCGGCCGCCTT
CCATGCGGTGGACCGGGCGGCGGCGGCCTTGGTGGCCCGCACCACCCAGGGGCTCTCGCCC
GCCGCCCTCACCCTCGCTTACATGGATTGGGCGATGCATCTGGCAGCGGCGCCGGGCAAGCA
GGCGGAGCTGGCGGTGAAGGCCACGCGCAAGGCGGCGCGCTTCTGGGCCTATGTGCTCGCC
TCCACCCTCGACCGCACCCAGGCGCCCTGCATCGCGCCCCTGGTGGGCGACGAACGGTTTTC
CGCCCCGGCCTGGCAGGACTGGCCCTACCGCTTCTGGTACCAGGCCTTTCTGCTCAATCAGC
AATGGTGGCACAACGCCACTCATGGCGTGCCGGGCGTGGCGCCGCACAATCAGGACGTGGT
GGCCTTCGCCGCCCGGCAGGTCCTGGACATGTTCTCCCCCTCCAACTCGCCCCTCACCAATCC
CGAGGTGGTGAAGAAGGCGCGCCAGACGCTAGGGGCCAATTTCGTCCAGGGCGCGCGCAAC
TTCATGGAGGACCAGTCGCGCAAAACCACCGGCCGCCCGCCCGTGGGCGCGGAGGCGTTCA
CTCCCGGCAAGGAGGTGGCCATCACCCCCGGCGAGGTGATCTACCGCAACCATCTCATCGAG
CTGATCCAATACCGGGCGACCACCCCTGACGTGCATGCGGAGCCGATCCTCATCGTGCCCGC
CTGGATCATGAAATATTACATCCTCGACCTGTCGCCCGATAACTCCCTGATCCGCTACCTGGT
GGACAAGGGGCACACGGTCTTCTGCATCTCCTGGCGCAATGTGAACGCGGAGGACCGCGATC
TCGGCTTCGAGGACTATCGCAAGATGGGCATCATGGCGGCCCTCGACGCGGTGAACGCGGT
GGTGCCGAACCAGAAGGTGCATGCGGTGGGTTATTGCCTGGGCGGCACGCTGCTCTCCATCG
CCGCCGCCGCCATGGCGCGGGTGGTCGACGACCGGCTGGGCTCCGTCACCCTGTTCGCCGC
CCAGACCGACTTCACCGAGCCCGGCGAACTGCAGCTCTTCGTGGACCCGAGCGAGCTTTATG
CCCTGGAAAGCCTCATGTGGGACCAGGGCTATCTCGGCGCCCGGCAGATGGCCGGCGCGTT
CGAGATGCTGCGCTCCAACGACCTCGTCTGGTCGCGCATGGTGCGCGACTATCTCATGGGCG
AGCGCGCACCCATGAACGACCTCATGGCCTGGAACGCAGATGCCACCCGCATGCCCTATCGC
ATGCATTCCCAGTATCTGCGCAACCTGTTCCTCGACAACGAGCTGGCCGTGGGCCGTTACATG
GTGGAGGGCCGGCCGGTGTCGCTGCAGAACATCCGCGTCCCGCTGTTCGTGGTGGGCACGG
AGCGGGACCACGTGGCGCCGTGGAAGTCGGTCTACAAGATCCACCAGCTCACCGACACGGA
CGTGACCTTCGTCCTCGCCTCCGGCGGGCACAATGCGGGCATCGTCTCCGAGCCCGGCCACA
AGCACCGGCACTATCGCATCCACGACACCAAATTGGGCGAGATGCATGTGAGCCCGGAGGAA
TGGATGGAGGCGAACCGGTCGCAGGACGGGTCCTGGTGGCCGGCCTGGGAGGCATGGCTC
GCCGGCCAGTCCTCCGGCCGCATCGGCCTGCCGCCTCTGGGCGCGCCCGGCTACGAGGTGC
TGGGCCCGGCGCCCGGCACCTATGTGATGCAAAGGTGA
phaC1 amino acid sequence (SEQ ID NO: 62):
MSAAEETSTHAELRLPQDGVEHDVAAAEAAVDRSAGEPSGTQASAAPAEAAPSSAPVSAAAGET
QPQDDTPPQDPSSLSDGPGLSPPVTQPGAASGDFGGPEAMGDAFMPPVPEEPMEGMAPAPAISA
APASVPSAGWPEDAPSALLDAVDASGDLPAAAEGAATAPEPIFRELPMTAAPAAPAIANILEPVAE
ALSALGAVAVSRPQVQREFRAAPEHPPMPRMAPPQAAPEPAPAVPPKPEAAKPEAAKPEAAKPEA
AKPEVAKPDAAAPDAAKSSGKAERPSGAGDGSGTSGVNMEAFSRNLARLVEEGGKAMAAYLSP
REQGKTDDLADDIADAMKTVGQVVEYWVADPQRTVEAQSRLMGGYLSVWANTLKRLAGEEATP
VAAPDPKDARFKDAGWNDSPMFDALKQAYLVTSDWAQNMVDEAKGLDPHTKHKAEFLVRQIAN
AISPSNFVLTNPELIRETLHSSGENLVKGMQNLTADLMAGQGTLKIRQTDLSAFEVGRNLATTPGK
VIFENELMQLIQYEPTTETVKKTPVLIVPPWINKFYILDLTAEKSLIKWLVSQGLTVFTISWVNPDG
RLAAKGFDDYMRDGIMAALDAVAVASGERRAHAVGYCVGGTLLATTLAYMAATGDDRIASATFL
TTQIDFTHAGDLKVFVDESQLATIERKMKEMGYLEGSKMASAFNMLRSNDLIWPYVVNNYMKGK
APFPFDLLFWNSDSTRMPAANHSYYLRNCYLTNNIARGLAELAGLKIDVTKVSIPVYSLATREDHI
APANSVYIGANLLSGPVRYVLAGSGHIAGVVNPPAKMKYQYWADGPVGPSYEAWLAGAQEHKG
SWWPDWFNWFSFNHPEEVPARAIGGGRLAPIEDAPGRYVKERS
phaC2 amino acid sequence (SEQ ID NO: 63):
MEARKMPVSPPSSATILPLPVSAAPPSTAPAASLPATASSSTNKASPSAFPAAWARSFALPGLPAFL
CPDEEDFEEGSGPAAFHAVDRAAAALVARTTQGLSPAALTLAYMDWAMHLAAAPGKQAELAVKA
TRKAARFWAYVLASTLDRTQAPCIAPLVGDERFSAPAWQDWPYRFWYQAFLLNQQWWHNATH
GVPGVAPHNQDVVAFAARQVLDMFSPSNSPLTNPEVVKKARQTLGANFVQGARNFMEDQSRKT
TGRPPVGAEAFTPGKEVAITPGEVIYRNHLIELIQYRATTPDVHAEPILIVPAWIMKYYILDLSPDNS
LIRYLVDKGHTVFCISWRNVNAEDRDLGFEDYRKMGIMAALDAVNAVVPNQKVHAVGYCLGGTL
LSIAAAAMARVVDDRLGSVTLFAAQTDFTEPGELQLFVDPSELYALESLMWDQGYLGARQMAGA
FEMLRSNDLVWSRMVRDYLMGERAPMNDLMAWNADATRMPYRMHSQYLRNLFLDNELAVGRY
MVEGRPVSLQNIRVPLFVVGTERDHVAPWKSVYKIHQLTDTDVTFVLASGGHNAGIVSEPGHKH
RHYRIHDTKLGEMHVSPEEWMEANRSQDGSWWPAWEAWLAGQSSGRIGLPPLGAPGYEVLGP
APGTYVMQR

1. A variant of bacterial strain VTT-E-193585 comprising a genetic modification that reduces the bacterial production of polyhydroxyalkanoic acid (PHA) as compared to strain VTT-E-193585.

2. The variant according to claim 1, wherein the genetic modification reduces bacterial PHA synthase activity as compared to strain VTT-E-193585, preferably wherein PHA synthase activity has been reduced to less than 10%, such as less than 5%, for example less than 2%.

3. The variant according to claim 1, wherein the variant comprises a genetic modification reducing the expression level of phaC1 and/or the activity of the phaC1 enzyme.

4. The variant according to claim 1, wherein the variant comprises a genetic modification reducing the expression level of phaC2 and/or the activity of the phaC2 enzyme.

5. The variant according to claim 1, wherein the genetic modification is a gene disruption.

6. The variant according to claim 1, wherein the variant comprises gene disruptions of both phaC1 and phaC2.

7. The variant according to claim 1, wherein the variant is the bacterial strain deposited under number VTT-E-213595.

8. The variant according to claim 1, wherein the variant has retained the ability to grow using hydrogen gas as energy source and carbon dioxide as the only carbon source.

9. A variant Xanthobacter strain comprising a gene disruption of one or more genes encoding a PHA synthase.

10. A culture comprising the variant bacteria according to claim 1.

11. A process for the production of biomass, said process comprising culturing the variant bacteria of claim 1.

12. The process according to claim 11, comprising culturing the variant bacteria in continuous culture with hydrogen as energy source and an inorganic carbon source, wherein the inorganic carbon source comprises carbon dioxide.

13. A process for the production of biomass, said process comprising culturing a variant chemoautotrophic bacterial strain in continuous culture with hydrogen as energy source and an inorganic carbon source, wherein the inorganic carbon source comprises carbon dioxide and wherein said variant chemoautotrophic strain comprises a gene disruption of one or more genes encoding a PHA synthase.

14. The process according to claim 12, wherein dissolved oxygen in the culture is maintained between 5% and 10%.

15. The process according to claim 12, wherein ammonium, urea, nitrate and/or nitrogen gas is used as nitrogen source.

16. The process according to claim 12, wherein pH in the culture is maintained between 5.5 and 8.0, e.g. between 6.5 and 7.0, such as at 6.8.

17. The process according to claim 12, wherein said culture is grown at a temperature between 25° C. and 40° C., e.g. between 28° C. and 32° C., such as at 30° C.

18. The process according to claim 12, comprising the further step of harvesting biomass produced during the culture, optionally comprising a further step of drying the biomass.

19. A process for the production of protein, comprising performing the process according to claim 18 and a further step of isolating protein from said biomass, wherein the process results in a protein fraction and a fraction comprising non-protein components.

20. The process according to claim 18, comprising the further step of producing a food or feed product from said biomass, from said protein fraction or from said fraction comprising non-protein components.

21. A product, such as biomass, protein, or non-protein components obtained or obtainable by the process according to claim 11.

22. A food or feed product obtained or obtainable by the process according to claim 20.

23. A method for genetic modification of bacterial strain VTT-E-193585 comprising the steps of:
a) providing bacteria of strain VTT-E-193585 or a genetically-modified or mutated strain generated using bacterial strain VTT-E-193585, such as VTT-E-213595,
b) introducing a nucleic acid construct into said bacteria, wherein said nucleic acid construct comprises:
i) sequences encoding a selectable marker,
ii) optionally further sequences to be integrated into the bacterial genome,
iii) flanking sequences allowing homologous recombination with the bacterial genome, and,
c) selecting a genetically-modified strain on the basis of the selectable marker.

24. The method according to claim 23, wherein the selectable marker is a gene providing antibiotic resistance, such as kanamycin or tetracycline resistance.

25. A variant of bacterial strain VTT-E-193585 comprising a genetic modification wherein said genetic modification comprises the disruption of a bacterial gene with a selectable marker providing antibiotic resistance, such as kanamycin or tetracycline resistance.

26. A culture comprising the variant according to claim 25.

27. A process for the production of biomass, said process comprising culturing the variant bacteria of claim 25.

28. The process according to claim 27, comprising culturing the variant bacteria in continuous culture with hydrogen as energy source and an inorganic carbon source, wherein the inorganic carbon source comprises carbon dioxide.

29. The process according to claim 28, further comprising the features specified in any one of claims 14 to 18.

30. A process for the production of protein, comprising performing the process according to claim 27, and a further step of isolating protein from said biomass, wherein the process results in a protein fraction and a fraction comprising non-protein components.

31. The process according to claim 30, comprising the further step of producing a food or feed product from said biomass, from said protein fraction or from said fraction comprising non-protein components.

32. A product, such as biomass, protein, or non-protein components obtained or obtainable by the process according to claim 27.

33. A food or feed product obtained or obtainable by the process according to claim 31.
